(19) United States
(12) Patent Application Publication
(10) Pub. No.: US 2005/0123536 A1
(43) Pub. Date: Jun. 9, 2005

(54) Treatment of immunological disorders using anti-dc30 antibodies

(76) Inventors: Che-Leung Law, North Shoreline, WA (US); Kerry Klussman, Seattle, WA (US); Alan F. Wahl, Mercer Island, WA (US); Peter Senter, Seattle, WA (US); Svetlana Doronina, Seattle, WA (US); Brian Toki, Everett, WA (US)

Correspondence Address: JONES DAY, 222 EAST 41ST ST, NEW YORK, NY, 10017, US

(21) Appl. No.: 10/496,628

(22) PCT Filed: Nov. 20, 2002
(86) PCT No.: PCT/US02/37223

Related U.S. Application Data
(60) Provisional application No. 60/331,750, filed on Nov. 20, 2001.

(51) Int. Cl. A61K 39/395

(52) U.S. Cl. 424/141.1; 424/144.1

(57) ABSTRACT

The present invention relates to methods for the treatment of immunological disorders other than cancer, comprising administering proteins characterized by their ability to bind to CD30 and exert a cytostatic or cytotoxic effect on an activated lymphocyte. Such proteins include monoclonal antibodies AC10 and IleFi1. AC10 and HeFi-1 derivatives, and antibodies that compete with AC10 and HeFi-1 for binding to CD30. Other such proteins include multivalent anti-CD30 antibodies and anti-CD30 antibodies conjugated to cytotoxic agents. Treatment modalities with antibodies of the invention are also provided.

This application claims benefit under 35 U.S.C. § 119(e) of U.S. provisional application no. 60/331,750, filed Nov. 20, 2001, which is incorporated by reference herein in its entirety.

### 1. FIELD OF THE INVENTION

This invention relates to methods and compositions for inducing cell death or stasis in activated lymphocytes using CD30-binding proteins, and applications for these methods and compositions for the treatment of immunological diseases such as autoimmunity, allergy, chronic inflamatory reactions, and graft versus host disease (GVHD). The present invention further relates to methods and compositions for treatment of immunological disorders by eliminating CD30-expressing lymphocytes using conjugates of CD30-binding proteins and cytotoxic agents. Exemplary CD30-binding proteins that are useful in the methods and compositions of the present invention include the anti-CD30 antibodies AC10 and HeFi-1 and conjugates of AC10 or HeFi-1 and cytotoxic agents.

### 2. BACKGROUND OF THE INVENTION

2.1 Lymphocytes

Subsequent to antigenic stimulation and cellular expansion, naïve T cells can develop into phenotypically distinct effector cells. Effector T cells (helper or cytotoxic T cells) that secrete pro-inflamatory cytokines like IFNγ and lymphotoxin are collectively designated as Th₁ or Tc₁ cells, and effector T cells that secrete IL-4, IL-5, IL-6, IL-9, IL-10, and IL-13 are known as Th₂ or Tc₂. Th₁/Tc₁ cytokines induce cell-mediated responses including the activation of CTL and monocyte/macrophages, whereas Th₂/Tc₂ cytokines induce humoral immune responses by enhancing antibody production by B cells. As a result, uncontrolled Th₁/Tc₁ responses are usually manifested as organ-specific autoimmune responses such as rheumatoid arthritis and diabetes. On the other hand, uncontrolled Th₂/Tc₂ responses are usually associated with allergic reactions and systemic autoimmune diseases like systemic lupus erythematosus. The two polarized T cell subsets can modulate each others activity in a reciprocal fashion—the presence of Th₂/Tc₂ cytokines can partially alleviate symptoms resulted from cell-mediated autoimmunity and vice versa (Seder and Mosmann, 1999, in ‘Fundamental Immunology’, 4^{th} Ed., pp 879-908; O'Gara and Arai, 2000, Trends Cell Biol. 10:542-550).

2.2 CD30

The leukocyte activation marker CD30 is a 105-120 kDa integral membrane glycoprotein and a member of the tumor necrosis factor receptor (TNF-R) superfamily. This family of key immunoregulatory molecules includes CD27, CD40, CD95, 0×40, TNF-R1 and TNF-R2. Originally identified on Reed-Sternberg cells in Hodgkin's disease (HD) using the Ki-1 monoclonal antibody (mAb) (Stein et al., 1985, Blood, 66, 848-858; Laudewitz et al, 1986, J. Invest. Dermatol. 86:350-354), CD30 has subsequently been found on anaplastic large cell lymphoma (ALCL), subsets of non-Hodgkin's lymphomas (NHL), as well as in rare solid tumors such as embryonal carcinomas and seminomas (Chiarle et al., 1999, Clin. Immunol. 90:157-164). Under normal conditions expression of CD30 is restricted to activated T and B cells and absent from resting lymphocytes, resting monocytes and from normal cells outside of the immune system. CD30 is expressed at high levels by activated cells in autoimmune disease, and shed CD30 from these cells is detectable in the circulation of patients suffering from diseases including rheumatoid arthritis (Gerli et al., 2000, J. Immunol. 164, 4399-4407), multiple sclerosis (McMillan et al. 2000, Acta Neurol. Scand. 101 :239-243) and systemic sclerosis (Ihn et al., J. Rheumatol. 27:698-702). Histological examination for CD30 expression identifies rare, large lymphoid cells in sections of lymph node, tonsil, thymus, and decidual endometrial cells at the placental interface (Durkop et al., 2000, J. Pathol. 190:613-618). CD30 is transiently expressed on T cells in culture after mitogen activation or antigen receptor crosslinking (Horle and Watanabe, 1998, Sem. Immunol. 10:457-470), and is constitutively expressed following some viral infections, e.g., HIV (Romagnani et al., 1996, Immunol. Lett. 51:83-88).

A ligand for CD30 (CD30L) has been identified. CD30L is a type II transmembrane protein, and it shares sequence and structural homologies with members of the TNF superfamily (Smith et al., 1993, Cell, 73′. 1349-1360). It is believed that CD30L, like TNFα, also exists as a trimer, and CD30L-CD30 interaction induces trimerization of CD30 to initiate signal transduction to CD30⁺ cells. Expression of CD30L has been demonstrated in activated T cells, neutrophils, eosinophils, resting B cells, epithelial cells and Hassal's corpuscles of the thyimic medulla, and some leukemic cells (Younes et al. Br. J. Haematol., 93:569-571: Pinto et al., 1996, Blood. 88:3'299-3305: Grüss et al., 1996, Am. J. Pathol. 149, 469-481; Gattei et al., 1997, Blood, 89, 2048-2059; Romagnani et al. 1998. Blood 91:3323-3332; Wiley et al., 1996. J. Immunol., 157:3635-3639).

The lymphocyte surface antigen CD30 is an activation marker transiently displayed on activated B and T cells and constitutively expressed on some malignant hematologic cells and on chronically activated-cells in several autoimmune diseases. Its role in lymphocyte regulation is believed to be one of attenuation, as lack of CD30 in knock-out animals results in hyperresponsiveness to immune stimuli, whereas over expression of the transgene in the thymus results in increased thymocyte depletion. Thus, elimination or attenuation of activated lymphocytes via CD30-targeted therapy could be efficacious in controlling autoimmune and chronic inflammatory diseases.

The complexity of CD30 in regulating lymphocyte survival has lead to disparate accounts of its effects in model systems. Duckett and Thompson (1997, Genes Dev. 11:2810-21) have shown the initial effect of CD30 signaling is to promote survival by recruitment of TRAF1 and TRAF2. This signal transduction complex promotes growth and survival-gene expression via NF-κB. Following this activation, TRAFs complexed with CD30 are then degraded, compromising the ability of the cells to further activate NF-κB. Consequently, the cells become sensitized to apoptosis induced by ligation of TNF-R1 (Duckett and Thompson, 1997, Genes Dev. 11:2810-21; Arch et al., 2000, Biochem. Biophys. Res. Commun. 272:936-945). Sensitization is dependent on a TRAF2 binding site within the cytoplasmic domain of CD30 and cellular degradation of TRAF2 is coincident with the onset of apoptosis.

2.3 Role of CD30 in the Immune System

2.3.1 Normal Immune System

The in vivo function of CD30 is not clearly understood. Ligation of CD30 in vitro has been shown to induce either cellular proliferation or growth inhibition (Gruss et al., 1994, Blood. 83, 2045-2056). Ligation of CD30 on T cells has been demonstrated to regulate a variety of in vitro T cell functions. Anti-CD30 mAbs co-stimulate with T cell antigen receptor ligation to augment cellular proliferation in T cells that have been primed by either antigens (Del Prete et al., 1995, J. Exp. Med., 182:1655-1661) or anti-CD3 antibodies (Gilfillan et al., 1998, J. Immunol. 160:2180-2187). CD30 signaling also promotes cytokine production in T cells. Expression of interleukin (IL)-2. IL-4, IL-5, IFNγ, and TNFα are enhanced by CD30 signals in PHA activated peripheral T cells (Grüss and Hermann, 1996, Leukemia Lymphoma, 20:397-409), T helper (Th) clones (Del Prete et al., 1995, J. Exp. Med., 182:1655-1661; Bengtsson et al., 2000, Scand. J. Immunol. 5′, 595-601), cytotoxic T cell (Tc/CTL) lines (Bowen et al., 1996, J. Immunol. 156:442-449), and γδ T cells (Biswas et al., 2000, Eur. J. Immunol. 30:2172-2180). CD30 may also play an important role in stimulating HIV replication. Cross-linking of CD30 by mAbs induces NF-κB activation and HIV production by a T cell line chronically infected with HIV (Biswas et al., 1995, Immunity, 2, 587-596). CD4⁺, HIV-infected T cells derived from patients also respond to anti-CD30 mAbs or CD30L stimulation to produce HIV (Maggi et al., 1995, Immunity 3:251-255).

CD30 has been implicated in the activation-induced cell death of T cells. Ligation of a CD8-CD30 chimera expressed in a T cell hybridoma enhanced apoptosis mediated by the T cell antigen receptor (Lee et al., 1996, J. Exp. Med. 183:669-674). In a second model of agonist withdrawal induced apoptosis in murine CD8 T cells, blockage of CD30 signaling using a CD30-Ig fusion protein partially prevented CD8 T cells from undergoing apoptosis (Telford et al., 1997, Cell. Immunol. 182: 125-136). Results obtained from animal models also suggest a role of CD30 in the induction of apoptosis in thymocytes, a negative selection process for the deletion of auto-reactive T cells (Amakawa et al., 1996, Cell 84:551-562). In CD30^{-/-} null mice negative selection is severely diminished, giving rise to increased thymocyte numbers (Amakawa et al., 1996 Cell 84:551-562); one demonstrated consequence being that CD30-deficient CD8-positive T cells are orders of magnitude more autoreactive in their capacitor to cause autoimmune diabetes (Kurts et al., 1999, Nature 398:341-344). Conversely, overexpression of CD30 has been shown to promote programmed cell death in thymocytes, and hence enhances negative selection of auto-reactive T cells in the thymus (Chiarle et al., 1999, J. Immunol. 163:194-205), and prevent T cell autoresponses to non-lymphoid tissue in the periphery (Heath et al., 1999, Immunol. Rev. 169:23-29).

2.3.2 The Disease State

Because CD30 was demonstrated to be preferentially expressed on human T cell clones secreting Th₂ cytokines (Del Prete et al., 1995, FASEB J., 9, 81-86), and cross-linking of CD30 on T cells promoted the development of Th₂-like T cells (Del Prete et al., 1995, J. Exp. Med., 182, 1655-1661), it has been postulated that immunological disorders involving Th₂ cytokines may result from the dysregulation of CD30⁺ T cells.

The expression of CD30 has been shown to be increased or altered in a variety of autoimmune and inflammatory diseases including atopic allergy (atopic dermatitis, atopic asthma, rhinoconjunctivitis, allergic rhinitis), systemic lupus erythematosus, systemic sclerosis (scleroderma), graft versus host disease (GVHD), HIV and EBV infection, measles, Omenn's syndrome, ulcerative colitis, rheumatoid arthritis, multiple sclerosis, psoriasis, Hashimoto's thyroiditis, primary biliary cirrhosis, Sjogren's syndrome, Wegener's granulomatosis, and tuberculosis (Gruss et al., 1997, Immunol. Today 18:156-163; Horie and Watababe, 1998, Sem. Immunol. 10:457-470; Bengtsson, 2001, Allergy 56:593-603; Gerli et al., 2001, Trends Immunol. 22:72-77).

CD30+ infiltrating T cells have been found in lesions and inflamed sites of atopic dermatitis (Caproni et al., 1997, Allergy 52:1063-1070; Cavagni et al., 2000, Allergy Immunol. 121:224-228), atopic asthma (Blanco Quirós et al., 1999, Pediatr. Allergy Immunol. 10:235-240), GVHD (D'Elios et al., 1997, J. Lewukoc. Biol. 61:539-544), tuberculosis (Munk et al., 1997, Int. Immunol. 9:713-720), primary biliary cirrhosis (Harada et al., 1999, J. Gastroenterol. Hepatol. 14:1197-1202), measles (Vinante et al., 1999, Haematologica 84:683-689), rheumatoid arthritis (Gerli et al., 2000, J. Immunol. 164:4399-4407; Gerli et al., 1995, Clin. Exp. Immunol. 102:547-550), and psoriasis (Ferenczi et al., 2000, J. Autoimmun. 14:63-78). Although CD30 expression is strictly restricted to a small percentage of activated lymphocytes in normal situations, increased expression of CD30 mRNA has been reported in atopic asthma and allergic rhinitis (Esnault et al., 1996, Clin. Exp. Immunol. 106:67-72). Correlation between altered expression of CD30 and immune disorders is further underscored by the high plasma levels of soluble CD30 (sCD30) found in many of the aforementioned diseases (Gruss et al., 1997. Immunol. Today 18:156-163; Horie and Watababe, 1998, Sem. Immunol. 10:457-470; Bengtsson, 2001, Allergy 56:593-603; Gerli et al., 2001, Trends Immunol. 22:72-77). In some of these diseases high plasma levels of sCD30 are not accompanied with circulating CD30⁺ T cells, but expansion of CD30′ T cells in the disease tissue is believed to be a potential source of sCD30 (Mavilia, 1997, Am. J. Pathol. 151:1751-1758).

Several diseases have been demonstrated to involve both Th₂ cytokines and CD30⁺ T cells (D'Elios et al., 1997, J. Leukoc. Biol. 61:539-544). Most patients with atopic dermatitis show increased production of Th₂ cytokines with parallel increases in the serum IgE levels and augmented numbers of circulating eosinophils (Leung, 1995, J. Allergy Clin. Immunol. 96:302-319). An increased frequency of allergen-specific Th₂ cells producing IL-4, IL-5, and IL-13 can also be detected in the peripheral blood of atopic dermatitis patients (Kimura et al., 1998, J. Allergy Clin. Immunol., 101, 84-89). A correlation between CD30 expression and Th₂ cytokine production has been noted in atopic dermatitis patients which has led to the suggestion that CD30 expression in circulating T cells might serve as an in vivo marker for the Th₂-dominated condition (Yamamoto et al., 2000, Allergy, 55, 1011-1018). Allergen induced IL-4 production in atopic asthma correlates with CD30 expression in PBMC isolated from atopic asthma patients (Leonard et al., 1997, Am. J. Respir. Cell Mol. Biol., 17, 368-375). This has also been shown in bronchial alveolar lavage yδ T cells from asthma patients (Spinozzi et al., 1995, Mol. Med. 1:82.1-826). Likewise, in systemic sclerosis, a disease characterized by elevated serum levels of sCD30 and CD30 expression in skin lesions, IL-4 mRNA can be detected by in situ hybridization in lymphocytes infiltrating skin lesions while most CD4⁺ T cell clones generated from skin infiltrates also demonstrate a Th₂ cytokine profile (Mavalia etal., 1997, Am. J. Path. 15, 1751-1758). In patients infected with HIV, a disproportionately higher frequency of CD8⁺/CD30⁺ cells producing Th₂ cytokines could be detected in peripheral blood when compared to healthy donors (Manetti et al. 1994, J. Exp. Med. 180:2407-2411). The emergence of a Th₂-type immunity in HIV infected patients also appears to be correlated with the immunopathology associated with disease progression (Rizzardi et al., 1998, Clin. Exp. Immunol. 114:61-65).

In contrast to the Th₂-related diseases, the role of CD30⁺ in Th₁-related diseases is not as well defined. High levels of sCD30 are found in the CSF of patients with multiple sclerosis and the circulation of rheumatoid arthritis patients (McMillan et al., 2000, Acta Neurol. Scand. 101:239-243; Gerli et al., 2000, J. Immunol. 164:4399-4407). In these diseases, both Th₁ cytokine driven responses, high circulating levels of CD30 are associated with disease remission, suggesting that Th₂ cytokines contributed by CD30⁺ T cells may counteract the pathogenic activities of Th₁ cytokines (McMillan et al., 2000, Acta Neurol. Scand. 101:239-243; Gerli et al., 2000, J. Immunol. 164:4399-4407). More recent experiments examining CD30 expression on T cell subsets determined that upon cellular activation Th₁ cells also express CD30 (Hamman et al., 1996, J. Immunol. 156: 1387-1391; Bengtsson et al., 1995, J. Leukoc. Biol. 58:683-689). An agonistic anti-CD30 mAb can stimulate the production of the Th₁ cytokine IFNγ in CD30⁺ Th₁ cells (Bengtsson et al., 2000, Scad. J. Immunol. 52:595-601). In atopic dermatitis, evidence is available to demonstrate a shift from the predominance of Th₂ cytokine production by T cell infiltrating skin lesions to the co-expression of Th₂ and Th₁ cytokines during disease progression (Grewe et al., 1998, Immunol. Today 19:359). Hence CD30 T cells may also play a role in the production of Th₁ cytokines in Th₁-related diseases.

2.4 Immunotherapeutics

Sustained activation of lymphocytes against self-antigens or allergens and the failure to terminate ongoing immune responses subsequent to the clearance of antigens are the underlying causes of the pathologies seen in autoimmune diseases, allergic reactions, and chronic inflammatory reactions. A variety of therapeutic regimens including antimetabolites, steroids, and anti-inflammatory agents are available for the treatment of autoimmune, allergic, and inflammatory diseases. Although these drugs are efficacious in alleviating symptoms, none of them work by specifically eliminating the pathogenic cells and most of them have severe side effects on patients.

Elimination or attenuation of activated lymphocytes bearing CD30 could be efficacious in controlling autoimmune and chronic inflamatory diseases. However, despite the above evidence for a role of CD30 in immune disorders, targeting CD30 by mAb has not been demonstrated to be effective in treating such disorders. Agents that are capable of eliminating or attenuating CD30-bearing activated lymphocytes would be highly desirable in the treatment of immunological disorders.

### 3. SUMMARY OF THE INTENTION

The present invention is based on the surprising discovery of a novel activity associated with certain classes of anti-CD30 antibodies. The novel activity is the ability of anti-CD30 antibodies to kill or inhibit the growth of activated lymphocytes, in certain instances by signaling through the CD30 receptor pathway. In certain preferred embodiments, the antibodies of the invention are able to induce apoptosis or growth arrest of activated lymphocytes as monospecific antibodies, in the absence of conjugation to cytotoxic reagents, and/or in the absence of cells other than the CD30-expressing lymphocytes (e.g., in the absence of effector cells).

Accordingly, the present invention provides methods for the treatment of an immunological disorder in a subject, preferably wherein the immune disorder is not cancer, comprising administering to the subject, in an amount effective for said treatment, a pharmaceutical composition comprising (a) a first antibody that (i) immunospecifically binds CD30 and (ii) induces CD30 signaling in a lymphocyte and/or exerts a cytostatic or cytotoxic effect on an activated lymphocyte; and (b) a pharmaceutically acceptable carrier. The antibody can be human, humanized or chimeric. In one embodiment, the antibody is multivalent. In a preferred embodiment, the antibody competes for binding to CD30 with monoclonal antibodies AC10 or HeFi-1.

The present invention further provides methods for the treatment of an immunological disorder in a subject, preferably wherein the immune disorder is not cancer, comprising administering to the subject, in an amount effective for said treatment, a pharmaceutical composition comprising (a) a first antibody that (i) immunospecifically binds CD30 and (ii) induces CD30 signaling in a lymphocyte and/or exerts a cytostatic or cytotoxic effect on an activated lymphocyte; and (b) a pharmaceutically acceptable carrier, said methods further comprising administering an agent that enhances or potentiates the cytostatic or cytotoxic effect of the first antibody. In certain embodiments, the agent that potentiates the cytostatic or cytotoxic effect of the first antibody is a second antibody, a ligand that binds to a receptor or receptor complex expressed on activated lymphocytes, or an immunosuppressive agent. Examples of such reagents and methods of their use are further described infra.

The present invention thus provides methods for the treatment of an immunological disorder in a subject, preferably wherein the immune disorder is not cancer, comprising administering to the subject, in an amount effective for said treatment, a pharmaceutical composition comprising (a) a first antibody that (i) immunospecifically binds CD30 and (ii) induces CD30 signaling in a lymphocyte and/or exerts a cytostatic or cytotoxic effect on an activated lymphocyte; and (b) a pharmaceutically acceptable carrier; said methods further comprising administering a second antibody to the subject. In a preferred embodiment, the second antibody recognizes a non-CD30 receptor or receptor complex expressed on activated lymphocytes.

In certain specific embodiments, the present invention provides methods for the treatment of an immunological disorder in a subject, wherein the immunological disorder is not cancer, comprising administering to the subject, in an amount effective for said treatment, a pharmaceutical composition comprising (a) an antibody that (i) immunospecifically binds CD30 and (ii) competes for binding to CD30 with monoclonal antibody AC10 or HeFi-1; and (b) a pharmaceutically acceptable carrier.

In other specific embodiments, the present invention provides methods for the treatment of an immunological disorder in a subject, wherein the immunological disorder is not cancer, comprising administering to the subject, in an amount effective for said treatment, a pharmaceutical composition comprising (a) an antibody that (i) immunospecifically binds CD30 and (ii) comprises SEQ ID NO:2 (the heavy chain variable region of the anti-CD30 antibody AC10); and (b) a pharmaceutically acceptable carrier.

In other specific embodiments, the present invention provides methods for the treatment of an immunological disorder in a subject, wherein the immunological disorder is not cancer, comprising administering to the subject, in an amount effective for said treatment, a pharmaceutical composition comprising (a) an antibody that (i) immunospecifically binds CD30 and (ii) comprises one, two or all of: SEQ ID NO:4, SEQ ID NO:6 and SEQ ID NO:8 (the heavy chain CDRs of the anti-CD30 antibody AC10), or variants of SEQ ID NO:4, SEQ ID NO:6 and SEQ ID NO:8 that differ from SEQ ID NO:4, SEQ ID NO:6 and SEQ ID NO:8 by one, two or three amino acids; and (b) a pharmaceutically acceptable carrier.

In other specific embodiments, the present invention provides methods for the treatment of an immunological disorder in a subject, wherein the immunological disorder is not cancer, comprising administering to the subject, in an amount effective for said treatment, a pharmaceutical composition comprising (a) an antibody that (i) immunospecifically binds CD30 and (ii) comprises SEQ ID NO: 18 (the heavy chain variable region of the anti-CD30 antibody HeFi-1); and (b) a pharmaceutically acceptable carrier.

In other specific embodiments, the present invention provides methods for the treatment of an immunological disorder in a subject, wherein the immunological disorder is not cancer, comprising administering to the subject, in an amount effective for said treatment, a pharmaceutical composition comprising (a) an antibody that (i) immunospecifically binds CD30 and (ii) comprises one, two or all of: SEQ ID NO:20, SEQ ID NO:22 and SEQ ID NO:24 (the heavy CDRs of the anti-CD30 antibody HeFi-1), or variants of SEQ ID NO:20, SEQ ID NO:22 and SEQ ID NO:24 that differ from SEQ ID NO:20, SEQ ID NO:22 and SEQ ID NO:24 by one, two or three amino acids; and (b) a pharmaceutically acceptable carrier.

In yet other specific embodiments, the present invention provides methods for the treatment of an immunological disorder in a subject, wherein the immunological disorder is not cancer, comprising administering to the subject, in an amount effective for said treatment, a pharmaceutical composition comprising (a) an antibody that (i) immunospecifically binds CD30 and (ii) competes for binding to CD30 with monoclonal antibody AC10 or HeFi-1, wherein said antibody is conjugated to a cytotoxic agent; and (b) a pharmaceutically acceptable carrier.

In certain specific embodiment of the present invention, the anti-CD30 antibody is an agonistic antibody. In another specific embodiment of the present invention, the anti-CD30 antibody is not a non-agonistic antibody. In another specific embodiment, the anti-CD30 antibody does not block binding of CD30 ligand to CD30.

In a preferred embodiment, where a second antibody that recognizes a non-CD30 receptor or receptor complex is administered to the subject, such an antibody is capable of enhancing the cytotoxic or cytostatic effect of the CD30 antibody. While not bound by any theory, such a second antibody enhances the cytotoxic or cytostatic effect of the CD30 antibody by delivering a cytostatic or cytotoxic signal to the activated lymphocytes. Exemplary receptors or receptor complexes include an immunoglobulin gene superfamily member, a TNF receptor superfamily member, an integrin, a cytokine receptor, a chemokine receptor, a major histocompatibility protein, a lectin, or a complement control protein. Non-limiting examples of suitable immunoglobulin superfamily members are CD2, CD3, CD4, CD8, CD19, CD22, CD28, CD79, CD90, CD152/CTLA-4. PD-1, and ICOS. Non-limiting examples of suitable TNF receptor superfamily members are CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, and APO-3. Non-limiting examples of suitable integrins are CD11a, CD11b, CD11c, CD18, CD29, CD41, CD49a, CD49b, CD49c, CD49d, CD49e, CD49f, CD103, and CD104. Non-limiting examples of suitable lectins are C-type, S-type, and I-type lectin.

In certain preferred embodiments, antibodies useful in the present methods, i.e., antibodies that bind to CD30 and exert a cytostatic or cytotoxic effect on an activated lymphocyte, are bispecific antibodies. In a specific embodiment, the bispecific antibodies bind to both CD30 and a non-CD30 receptor or receptor complex expressed on activated lymphocytes. Preferably, the portion of the bispecific antibody that binds to the non-CD30 receptor or receptor complex is capable of enhancing the cytotoxic or cytostatic effect of the CD30 antibody. The non-CD30 binding portion of the bispecific antibody preferably enhances the cytotoxic or cytostatic effect of the CD30 antibody by delivering a cytostatic or cytotoxic signal to the activated lymphocytes. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein. Non-limiting examples of suitable immunoglobulin superfamily members are CD2, CD3, CD4, CD8, CD19, CD22, CD28, CD79, CD90, CD152/CTLA-4, PD-1, and ICOS. Non-limiting examples of suitable TNF receptor superfamily members are CD27, CD40, CD95/Fas, CD134/OX40, CD137/4-1BB, TNF-R1, TNFR-2, RANK, TACI, BCMA, osteoprotegerin. Apo2/TRAIL-R1, TRAIL-R1, TRAIL-R3, TRAIL-R4, and APO-3. Non-limiting examples of suitable integrins are CD11a, CD11b, CD11 c, CD18. CD29, CD41, CD49a, CD49b, CD49c, CD49d, CD49e, CD49f, CD103, and CD104. Non-limiting examples of suitable lectins are C-type, S-type, and I-type lectin.

In other embodiments, the therapeutic methods of the present invention further comprise administering to the subject a ligand that binds to a receptor or receptor complex expressed on activated lymphocytes, concurrently or successively with the anti-CD30 antibody. Preferably, the ligand is capable of enhancing the cytotoxic or cytostatic effect of the CD30 antibody, for example by delivering a cytostatic or cytotoxic signal to the activated lymphocytes. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein. Non-limiting examples of suitable immunoglobulin superfamily members are CD2, CD3, CD4, CD8, CD19, CD22, CD28, CD79, CD90, CD152/CTLA-4, PD-1, and ICOS. Non-limiting examples of suitable TNF receptor superfamily members are CD27, CD40, CD95/Fas, CD134/OX40, CD137/4-1BB, TNF-R1, TNFR-2, RANK, TACI. BCMA, osteoprotegerin, Apo2/TRAIL-R1, TRAIL-R2, TRAIL-R3, TRAIL-R4, and APO-3. Non-limiting examples of suitable integrins are CD11a, CD11b, CD11c, CD18, CD29, CD41, CD49a, CD49b, CD49c, CD49d CD49e, CD49f, CD103, and CD104. Non-limiting examples of suitable lectins are C-type, S-type, and I-type lectin. Ligands that bind to the foregoing receptors are known to those of skill in the art.

In certain specific embodiments of the present therapeutic methods, the anti-CD30 antibody is a fusion protein comprising the amino acid sequence of a second protein that is not an antibody. In a preferred embodiment, the second protein confers multivalent binding properties to the CD30 antibody.

In other embodiments, the therapeutic methods of the present invention further comprise administering to the subject a cytostatic, cytotoxic, and/or immunosuppressive agent. In one embodiment, the immunosuppressive agent is gancyclovir, acyclovir, etanercept, rapamycin, cyclosporine or tacrolimus. In other embodiments, the immunosuppressive agent is an antimetabolite, a purine antagonist (e.g. azathioprine or mycophenolate mofetil), a dihydrofolate reductase inhibitor (e.g., methotrexate), a glucocorticoid. (e.g., cortisol or aldosterone), or a glucocorticoid analogue (e.g., prednisone or dexamethasone). In yet other embodiments, the immunosuppressive agent is an alkylating agent (e.g., cyclophosphamide). In yet other embodiments, the immunosuppressive agent is an anti-inflamatory agent, including but not limited to a cyclooxygenase inhibitor, a 5-lipoxygenase inhibitor, and a leukotriene receptor antagonist.

In certain preferred embodiments, antibodies useful in the present methods, i.e., antibodies that bind to CD30 and induce CD30 signaling in a lymphocyte and/or exert a cytostatic or cytotoxic effect on an activated lymphocyte, are conjugated to a cytostatic, cytotoxic or immunosuppressive agent. Such conjugated antibodies are sometimes referred to herein as anti-CD30 antibody-drug conjugates (“ADC” or “ADCs”) or anti-CD30 antibody-cytotoxic agent/immunosuppressive agent conjugates.

In certain preferred embodiments, the cytotoxic agent is selected from the group consisting of an enediyne, a lexitropsin, a duocarmycin, a taxane, a puromycin, a dolastatin, a maytansinoid, and a vinca alkaloid. In certain, more specific embodiments, the cytotoxic agent is paclitaxel, docetaxel, CC-1065, SN-38, topotecan, morpholino-doxorubicin, rhizoxin, cyanomorpholino-doxorubicin, dolastatin-10, echinomycin, combretastatin, calicheamicin, maytansine, DM-1, auristatin E, AEB, AEVB, AEFP, MMAE, or netropsin. The structures of AEB, AEVB, AEFP and MMAE are depicted in Section 3.1, infra.

In other preferred embodiments, the cytotoxic agent of an anti-CD30 antibody-cytotoxic agent conjugate of the invention is an anti-tubulin agent. In more specific embodiments, the cytotoxic agent is selected from the group consisting of a vinca alkaloid, a podophyllotoxin, a taxane, a baccatin derivative, a cryptophysin, a maytansinoid, a combretastatin, and a dolastatin. In more specific embodiments, the cytotoxic agent is vincristine, vinblastine, vindesine, vinorelbine, VP-16, camptothecin, paclitaxel, docetaxel, epithilone A, epithilone B, nocodazole, coichicine, colcimid, estramustine, cemadotin, discodermolide, maytansine, DM-1, AEFP, auristatin E, AEB, AEVB, AEFP, MMAE or eleutherobin.

In a specific embodiment, the cytotoxic agent of an anti-CD3 G antibody-cytotoxic agent conjugate of the invention is MMAE. In another specific embodiment, the cytotoxic agent of an anti-CD30 antibody-cytotoxic agent conjugate of the invention is AEFP.

In specific embodiments, the anti-CD30 antibody of an anti-CD30 antibody-cytotoxic agent conjugate of the invention is conjugated to the cytotoxic agent via a linker, wherein the linker is peptide linker. In specific embodiments, the anti-CD30 antibody of an anti-CD30 antibody-cytotoxic agent conjugate of the invention is conjugated to the cytotoxic agent via a linker, wherein the linker is a val-cit linker, a phe-lys linker, a hydrazone linker, or a disulfide linker. In certain embodiments, the anti-CD30 antibody of an anti-CD30 antibody-cytotoxic agent conjugate of the invention is conjugated to the cytotoxic agent via a peptide linker.

In certain embodiments, the conjugate of the invention is anti-CD30-valine-citrulline-MMAE (anti-CD30-val-citMMAE or anti-CD30-vcMMAE) or anti-CD30-valine-citrulline-AEFP (anti-CD30-val-citAEFP or anti-CD30-vcAEFP). In specific embodiments, the conjugate of the invention is AC10-valine-citrulline-MMAE (AC10-val-citMMAE or AC10-vcMMAE) or AC10-valine-citrulline-AEFP (AC10-val-citAEFP or AC10-vcAEFP).

In other embodiments, the conjugate of the invention is anti-CD30-phenylalanine-lysine-MMAE (anti-CD30-phe-lysMMAE or anti-CD30-fkMMAE) or anti-CD30-phenylalanine-lysine-AEFP (anti-CD30-phe-lysAEFP or anti-CD30-fkAEFP). In specific embodiments, the conjugate of the invention is AC10-phenylalanine-lysine-MMAE (AC10-phe-lysMMAE or AC10-fkMMAE) or AC10-phenylalanine-lysine-AEFP (AC10-phe-lysAEFP or AC10-fkAEFP).

Thus, in a specific embodiment, the present invention provides methods for the treatment of an immunological disorder in a subject, wherein the immunological disorder is not cancer, comprising administering to the subject, in an amount effective for said treatment, a pharmaceutical composition comprising (a) cAC10-val-cit-MMAE; and (b) a pharmaceutically acceptable carrier.

In another specific embodiment, the present invention provides methods for the treatment of an immunological disorder in a subject, wherein the immunological disorder is not cancer, comprising administering to the subject, in an amount effective for said treatment, a pharmaceutical composition comprising (a) cAC10-val-cit-AEFP; and (b) a pharmaceutically acceptable carrier.

In certain embodiments, the anti-CD30 antibody of an anti-CD30 antibody-cytotoxic agent conjugate of the invention is conjugated to the cytotoxic agent via a linker, wherein the linker is hydrolyzable at a pH of less than 5.5. In a specific embodiment the linker is hydrolyzable at a pH of less than 5.0.

In certain embodiments, the anti-CD30 antibody of an anti-CD30 antibody-cytotoxic agent conjugate of the invention is conjugated to the cytotoxic agent via a linker, wherein the linker is cleavable by a protease. In a specific embodiment, the protease is a lysosomal protease. In other specific embodiments, the protease is, inter alia, a membrane-associated protease, an intracellular protease, or an endosomal protease.

In certain embodiments, the anti-CD30 antibody of the invention is a monoclonal antibody, a humanized chimeric antibody, a chimeric antibody, a humanized antibody, a glycosylated antibody, a multispecific antibody, a human antibody, a single-chain antibody, a Fab fragment, a F(ab′) fragment, a F(ab′)₂ fragment, a Fd, a single-chain Fv, a disulfide-linked Fv, a fragment comprising a V_{L} domain, or a fragment comprising a V_{H} domain. In certain embodiments, the anti-CD30 antibody of an anti-CD30 antibody-cytotoxic agent conjugate of the invention is a polypeptide that binds specifically to CD30. In certain embodiments, the antibody is a bispecific antibody. In other embodiments, the antibody is not a bispecific antibody.

In certain embodiments, the anti-CD30 antibody is radioactively labeled. In certain embodiments, the anti-CD30 antibody of the anti-CD30 antibody-cytotoxic agent conjugate is radioactively labeled. In specific embodiments, the radioactive label is ⁹⁰Y, ¹¹¹In, ²¹¹At, ¹³¹I, ²¹²Bi, ²¹³Bi, ²²⁵Ac, ¹⁸⁶Re, ¹⁸⁵Re, ¹⁰⁹Pd, ⁶⁷Cu, ⁷⁷Br, ¹⁰⁵Rh, ¹⁹⁸Au, ¹⁹⁹Au or ²¹²Pb.

Immunological disorders encompassed by the methods of the present invention are Th₂-lymphocyte related disorders (e.g., atopic dermatitis, systemic lupus erythematosus (“SLE”), atopic asthma, rhinoconjunctivitis, allergic rhinitis, Omenn's syndrome, systemic sclerosis, and chronic graft versus host disease); Th₁ lymphocyte-related disorders (e.g., rheumatoid arthritis, multiple sclerosis, psoriasis, Sjorgren's syndrome, Hashimoto's thyroiditis, Grave's disease, primary biliary cirrhosis, Wegener's granulomatosis, tuberculosis and acute graft versus host disease); viral infection-related disorders (e.g., Epstein-Barr virus, human immunodeficiency virus, human T leukemia virus, hepatitis B virus, and measles virus infections); and activated B lymphocyte-related disorders (e.g., systemic lupus erythematosus, Goodpasture's syndrome, rheumatoid arthritis, and tripe I diabetes).

3.1 Abbreviations

The abbreviation “AEFP” refers to dimethylvaline-valine-dolaisoleuine-dolaproine-phenylalanine-p-phenylenediamine, the auristatin E derivative depicted below:

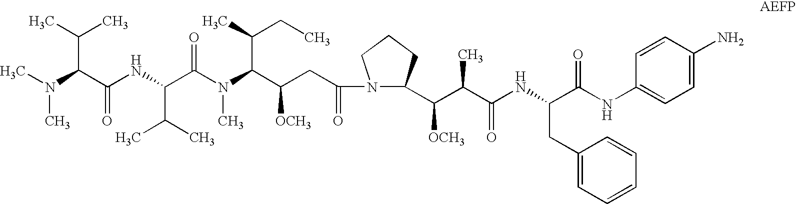

The abbreviation “MMAE” refers to monomethyl auristatin E, the auristatin E derivative depicted below:

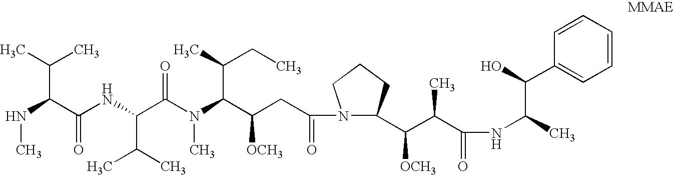

The abbreviation “AEB” refers to an ester produced by reacting auristatin E with paraacetyl benzoic acid, the structure of which is depicted below:

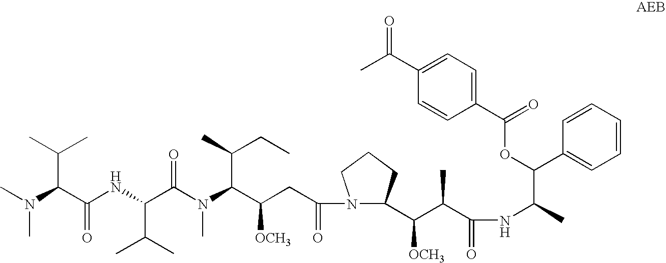

The abbreviation “AEVB” refers to an ester produced by reacting auristatin E with benzoylvaleric acid, the structure of which is depicted below:

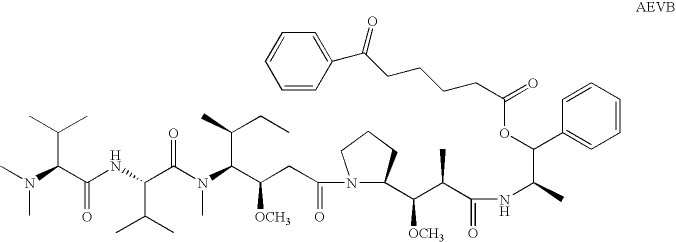

The abbreviations “fk” and “phe-lys” refer to the linker phenylalanine-lysine.

The abbreviations “vc” and “val-cit” refer to the linker valine-citrulline.

### 4. BRIEF DESCRIPTION OF THE FIGURES

FIG. 1. Expression of CD30 on Jurkat T cells: The Jurkat T cell line was examined for the expression of CD3, CD4, CD28, and CD30 by flow cytometric analysis.

FIG. 2. Effect of anti-CD30 on the proliferation of Jurkat T cell: Jurkat T cells were incubated with graded doses of a chimeric AC10 (cAC10) anti-CD30 mAb with (XL cAC10) or without (cAC10) the presence of a secondary cross-linking goat anti-human (GAH) Fcγ specific Ab. Proliferation was assessed by a pulse of tritiated thymidine (³H-TdR) during the last 4 hours of a 72 hour incubation.

FIG. 3. Secondary cross-linking of anti-CD30 mAb on the Jurkat T cells inhibited proliferation: Jurkat T cells were incubated with graded doses of the AC10 or HeFi-1 mAbs in the presence of a secondary cross-linking goat anti-mouse (GAM) Fcγ specific Ab at different primary to secondary Ab ratios as indicated in the figure. Proliferation was assessed by a pulse of ³H-TdR during the last 4 hours of a 48-hour incubation.

FIG. 4. Secondary cross-linking of anti-CD30 mAb on the Jurkat T cells induced apoptosis: Jurkat T cells were treated with 0.2 μg/ml of either AC10 or HeFi-1 cross-linked by 0.8 μg/ml of a GEM secondary Ab. Cell cycle disposition and DNA sysnthesis were detected by propidium iodide (PI) and anti-bromodeoxyuridine (BrdU) staining after 24 and 48 hours of treatment.

FIG. 5. Secondary cross-linking of anti-CD30 mAb on the Jurkat T cells induced apoptosis: Jurkat T cells were treated with graded doses of either AC10 or HeFi-1 cross-linked by a GAM secondary Ab at a primary to secondary Ab concentration ratio of 1:4. Cell cycle disposition and DNA synthesis were detected by PI and anti-BrdU staining after 24 and 48 hours of treatment.

FIG. 6. Anti-CD3 mAb enhanced anti-CD30-induced apoptosis in Jurkat T cells: Jurkat T cells were treated with AC10, the anti-CD3 mAb OKT3, or a combination of both mAbs at graded doses as indicated in the figure. Four-fold excess of a GAM secondary Ab was used to cross-link the primary mAbs. Cell cycle disposition and DNA sysnthesis were detected by PI and anti-BrdU staining after 24 and 48 hours of treatment.

FIG. 7. Detection of anti-CD30-induced apoptosis in Jurkat T cells by Annexin V binding: Jurkat T cells were treated with AC10, HeFi-1, the anti-CD3 mAb OKT3, or a combination of AC10 or HeFi-1 and OKT3. Anti-CD30 and anti-CD3 mAbs were used at 2 μg/ml. GAM secondary Ab was used in 10-fold excess to cross-link the primary mAbs. Binding of FITC-conjugated Annexin V enabled the detection of cells undergoing apoptosis. Membrane permeability to PI was used to detect dead cells that had lost membrane integrity. In the figure, Annexin V⁻/PI⁻ events represent live cells (lower left quadrant), Annexin V⁺/PI⁻ events represent apoptotic cells (lower right quadrant), and Annexin V⁺/PI⁺ events represent dead cells (upper right quadrant). Numbers outside of the density plots denote the percentage of cells present in each of the quadrants.

FIG. 8. Chemical structures of the antibody drug conjugates (ADCs) cAC10-vcMMAE, cAC10-fkMMAE, cAC10-vcAEFP, and cAC10-fkAEFP.

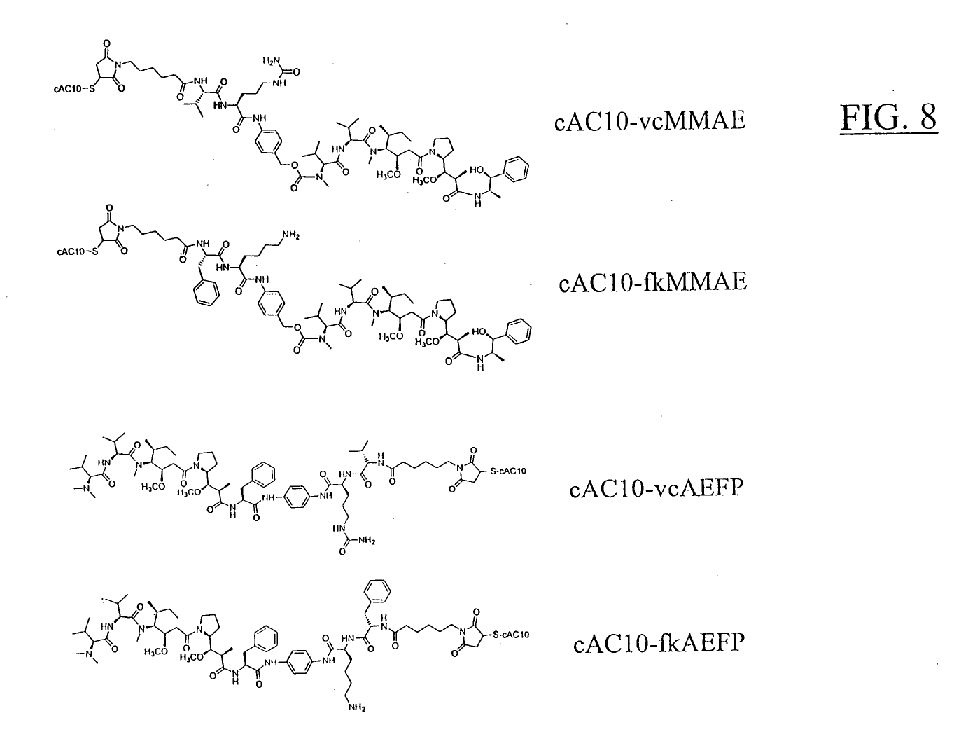

FIG. 9. Growth inhibitory effect of the cAC10-vcMMAE conjugate on the proliferation of Jurkat T cells: Graded doses of the cAC10-vcMMAE conjugate or a non-binding control IgG (cIgG)-vcMMAE conjugate were added to Jurkat T cells at the initiation of culture. Cells were exposed to the ADCs continuously for a total of 96 hr. Proliferation was assessed by a pulse of H-TdR during the last 16 hours of incubation.

FIG. 10. CD30 induction on activated normal peripheral blood mononuclear cells (PBMC): PBMC from normal donors were stimulated with either anti-CD3, anti-CD3+ anti-CD28, or in medium alone (negative control). On days 0, 2, 4, 6, and 8 cells were harvested and expression of CD30 was determined on both CD4 cells (shown) and CD8 cells (not shown) by multi-color flow cytometric analysis.

FIG. 11. Growth inhibitory effects of cAC10 ADCs on activated normal human PBMC: PBMC from normal donors were activated with anti-CD3+anti-CD8 mAbs as described in FIG. 10. Graded doses of different ADCs as indicated in the figure were added to the cells at the initiation of culture. Cells were exposed to the ADCs continuously for a total of 48 or 72 hr. Proliferation was assessed by incorporation of ³H-TdR during the last 16 hours of incubation.

FIG. 12. Induction of CD30 on memory and naïve T cells: Peripheral blood T lymphocytes, memory T lymphocytes, and naïve T lymphocytes were enriched from PBMC using immuno-selection. T cells were then activated by anti-CD3+anti-CD28 mAbs for 72 hr in the presence of both recombinant human interleukin (rhIL)-2 and rhIL-4. Expression of CD30 on CD4⁺ and CD8⁺ cells was then assessed by flow cytometry.

FIG. 13. Growth inhibitory effects of cAC10 ADCs on activated memory and naïve T lymphocytes: Memory and naïve T cells enriched from PBMC were induced to express CD30 as described in FIG. 12. After 72 hr of induction, T cells were harvested and treated with graded doses of different ADCs as indicated in the figure in the presence of rhIL-2. Cells were exposed to the ADCs continuously for an additional 48 or 72 hr. Proliferation was assessed by incorporation of ³H-TdR during the last 16 hours of incubation.

FIG. 14. CD30 induction on T cells stimulated by allogeneic cells: CD4-enriched PBMC were stimulated with successive cycles of irradiated allogeneic Burkitt's lymphoma Daudi cells. Expression of CD30 on CD4 cells was determined by flow cytometric analysis 3-5 days and 7-9 days after the addition of the allogeneic stimulator cells.

FIG. 15. Generation of T lymphocyte clones: a schematic to summarize the generation of T cell clones from PBMC.

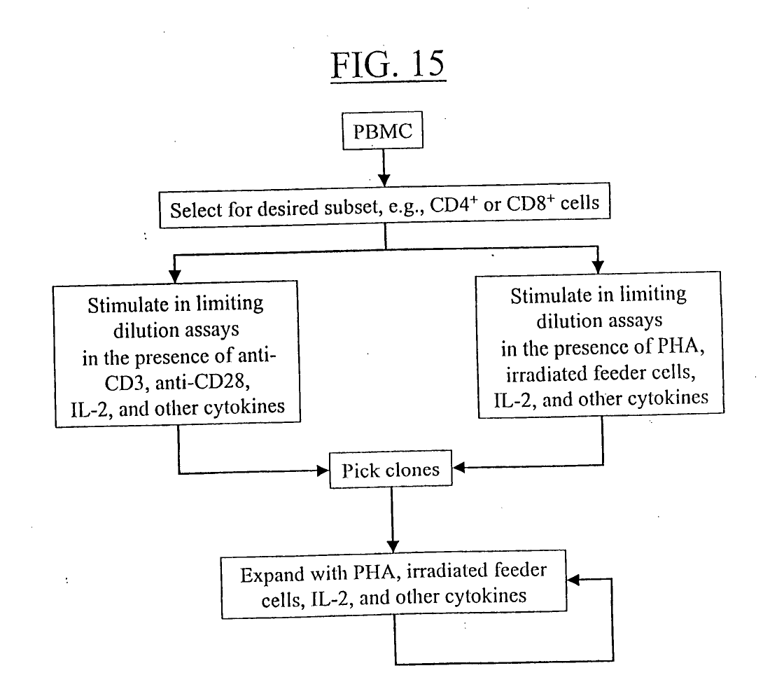

FIG. 16. Phenotype of T lymphocyte clones: Ten independent T lymphocyte clones isolated from 3 different normal donors according to the scheme depicted in FIG. 15 were examined for their surface expression of CD3, CD4, CD8, CD28, and CD30 by flow cytometric analysis. Histograms from two representative clones are shown, and the levels of receptor expression in all 10 clones indicated by the mean fluorescence intensities obtained from flow cytometric analysis were tabulated.

FIG. 17. Cytokine expression by T lymphocyte clones: T lymphocyte clones depicted in FIG. 16 were examined for their ability of express IL-2, 4, 5, 13, IFNγ, and TNFα by flow cytometric analysis. Histograms from two representative clones are shown, and the levels of cytokine expression in all 10 clones indicated by the mean fluorescence intensities obtained from flow cytometric analysis were tabulated. T lymphocytes clones were also assigned to different subsets based on cytokine expression in the bottom row of the table

FIG. 18. Upregulation of CD30 upon stimulation of T lymphocyte clones: A resting Th₂ clone and a resting Tc₂ clone representing the panel shown in FIGS. 16 and 17 were stimulated with phytohemaggutinin (PHA), irradiated feeder cells, IL-2, and IL-4. CD25 and CD30 expression on days 0, 2, 4, and 7 were determined by flow cytometric analysis.

FIG. 19. Growth inhibitory effects of cAC10 ADCs on the T lymphocte clone 40E10: 40E10 cells were induced to express CD30 as described in FIG. 18. At the peak of CD30 expression on day 2 of activation, cells were harvested and treated with graded doses of different ADCs as indicated in the figure in the presence of rhIL-2 and rhIL-4. Cells were exposed to the ADCs continuously for an additional 48 or 72 hr. Proliferation was assessed by incorporation of ³H-TdR during the last 16 hours of incubation.

FIG. 20. Growth inhibitory effects of cAC10 ADCs on the T lymphocyte clone 40H7: 40H7 cells were induced to express CD30 as described in FIG. 1S. At the peak of CD30 expression on day 2 of activation, cells were harvested and treated with graded doses of different ADCs as indicated in the figure in the presence of rhIL-2 and rhIL-4. Cells were exposed to the ADCs continuously for an additional 48 or 72 hr. Proliferation was assessed by a pulse of ³H-TdR during the last 16 hours of incubation.

FIG. 21. Inhibition of proliferation in T cell clones induced by cAC10 ADCs was accompanied by apoptosis induction: Clone 40E10 and 40H7 were incubated with 1 μg/ml of different ADCs as indicated in the figure. After 48 hr of incubation, Annexin V binding and permeability of PI were used to assess the extent of apoptosis induction.

FIG. 22. Growth inhibitory effect of cAC10 ADCs on the T lymphocyte clones 3.27.2 and 4.01.1: 3.27.2 and 4.01.1 cells were induced to express CD30 as described in FIG. 18. At the peak of CD30 expression on day 2 of activation, cells were harvested and treated with graded doses of different ADCs as indicated in the figure in the presence of rhIL-2. Cells were exposed to the ADCs continuously for an additional 72 hr. Proliferation was assessed by a pulse of ³H-TdR during the last 4 hours of incubation.

FIG. 23. A summary of the efficacies of cAC10 ADCs to inhibit the proliferation of CD30⁺ T lymphocyte clones and activated normal T lymphocytes.

### 5. DETAILED DESCRIPTION OF THE INVENTION

The present invention relates to the use of proteins that bind to CD30 and induce CD30 signaling in a lymphocyte and/or exert a cytostatic or cytotoxic effect on activated lymphocytes to treat or prevent immunological disorders. The invention further relates to proteins that compete with AC10 or HeFi-1 for binding to CD30 and induce CD30 signaling in a lymphocyte and/or exert a cytostatic or cytotoxic effect on activated lymphocytes. In one embodiment, the protein is an antibody. In a preferred mode of the embodiment, the antibody is AC10 or HeFi-1, most preferably a humanized or chimeric form of AC10 or HeFi-1.

In certain embodiments, the anti-CD30 antibodies of the present invention are capable of inducing apoptosis or growth arrest of activated lymphocytes as monospecific antibodies, in the absence of conjugation to cytotoxic reagents (e.g. small molecules, toxins, radioactive isotopes), and/or in the absence of cells other than the CD30-expressing lymphocytes (e.g., in the absence of effector cells such as natural killer cells). Without being bound by any theory, the present inventors believe that such antibodies induce apoptosis or growth arrest by signaling through the CD30 pathway.

The invention further relates to the use of proteins encoded by and nucleotide sequences of AC10 and HeFi-1 genes to treat or prevent immunological disorders. The invention further relates to fragments and other derivatives and analogs of such AC10 and HeFi-1 proteins. Nucleic acids encoding such fragments or derivatives are also within the scope of the invention. Production of the foregoing proteins, e.g., by recombinant methods, is provided.

The invention also relates to the use of AC10 and HeFi-1 proteins and derivatives including fusion/chimeric proteins which are functionally active, i.e., which are capable of displaying binding to CD30 and exerting a cytostatic or cytotoxic effect on activated lymphocytes, to treat or prevent immunological disorders.

Antibodies to CD30 whose use is encompassed by the invention include human, chimeric or humanized antibodies, and such antibodies conjugated to cytotoxic agents such as chemotherapeutic drugs.

The invention further relates to methods of treating or preventing immunological disorders comprising administering a composition comprising a protein or nucleic acid of the invention alone or in combination with a cytotoxic agent, including but not limited to a chemotherapeutic drug.

For clarity of disclosure, and not by way of limitation, the detailed description of the invention is divided into the subsections which follow.

5.1 Proteins of the Invention

The present invention encompasses the use of proteins, including but not limited to antibodies, that bind to CD30 and exert cytostatic and/or cytotoxic effects on activated lymphocytes, for the treatment of an immunological disorder. The invention further relates to the use of proteins that compete with AC10 or HeFi-1 for binding to CD30 and exert a cytostatic or cytotoxic effect on activated lymphocytes for the treatment of immunological disorders.

The present invention further encompasses the use of proteins comprising a CDR of HeFi-1 (SEQ ID NO:20, SEQ ID NO:22; SEQ ID NO:24; SEQ ID NO:28, SEQ ID NO:30 or SEQ ID NO:32) or AC10 (SEQ ID NO:4; SEQ ID NO:6; SEQ ID NO:8; SEQ ID NO:12; SEQ ID NO:14; or SEQ ID NO:16) for the treatment of an immunological disorder.

The present invention further encompasses the use of proteins comprising a variable region of HeFi-1 (SEQ ID NO: 18 or SEQ ID NO:26) or AC10 (SEQ ID NO:2 or SEQ ID NO: 10) for treating an immunological disorder. A table indicating the region of AC10 or HeFi-1 to which each SEQ ID NO corresponds to is provided below:

TABLE 1

| | NUCLEOTIDE OR | |
|---|---|---|
| MOLECULE | AMINO ACID | SEQ ID NO |
| AC10 Heavy Chain Variable Region | Nucleotide | 1 |
| AC10 Heavy Chain Variable Region | Amino Acid | 2 |
| AC10 Heavy Chain-CDR1(H1) | Nucleotide | 3 |
| AC 10 Heavy Chain-CDR1(H1) | Amino Acid | 4 |
| AC 10 Heavy Chain-CDR2(H2) | Nucleotide | 5 |
| AC 10 Heavy Chain-CDR2(H2) | Amino Acid | 6 |
| AC 10 Heavy Chain-CDR3(H3) | Nucleotide | 7 |
| AC 10 Heavy Chain-CDR3(H3) | Amino Acid | 8 |
| AC 10 Light Chain Variable Region | Nucleotide | 9 |
| AC 10 Light Chain Variable Region | Amino Acid | 10 |
| AC 10 Light Chain-CDR1(L1) | Nucleotide | 11 |
| AC 10 Light Chain-CDR1(L1) | Amino Acid | 12 |
| AC 10 Light Chain-CDR2(L2) | Nucleotide | 13 |
| AC 10 Light Chain-CDR2(L2) | Amino Acid | 14 |
| AC 10 Light Chain-CDR3(L3) | Nucleotide | 15 |
| AC 10 Light Chain-CDR3(L3) | Amino Acid | 16 |
| HeFi-1 Heavy Chain Variable Region | Nucleotide | 17 |
| HeFi-1 Heavy Chain Variable Region | Amino Acid | 18 |
| HeFi-1 Heavy Chain-CDR1(H1) | Nucleotide | 19 |
| HeFi-1 Heavy Chain-CDR1(H1) | Amino Acid | 20 |
| HeFi-1 Heavy Chain-CDR2(H2) | Nucleotide | 21 |
| HeFi-1 Heavy Chain-CDR2(H2) | Amino Acid | 22 |
| HeFi-1 Heavy Chain-CDR3(H3) | Nucleotide | 23 |
| HeFi-1 Heavy Chain-CDR3(H3) | Amino Acid | 24 |
| HeFi-1 Light Chain Variable Region | Nucleotide | 25 |
| HeFi-1 Light Chain Variable Region | Amino Acid | 26 |
| HeFi-1 Light Chain-CDR1(L1) | Nucleotide | 27 |
| HeFi-1 Light Chain-CDR1(L1) | Amino Acid | 28 |
| HeFi-1 Light Chain-CDR2(L2) | Nucleotide | 29 |
| HeFi-1 Light Chain-CDR2(L2) | Amino Acid | 30 |
| HeFi-1 Light Chain-CDR3(L3) | Nucleotide | 31 |
| HeFi-1 Light Chain-CDR3(L3) | Amino Acid | 32 |

The present invention further comprises the use of functional derivatives or analogs of AC10 and HeFi-1 for treating immunological disorders. As used herein, the term “functional” in the context of a peptide or protein of the invention indicates that the peptide or protein is 1) capable of binding to CD30 and 2) induces CD30 signaling in a lymphocyte and/or exerts a cytostatic and/or cytotoxic effect on activated lymphocytes.

Generally, antibodies suitable for practicing the methods of the present invention immunospecifically bind CD30 and induce CD30 signaling in a lymphocyte and/or exert cytostatic and cytotoxic effects on activated lymphocytes. Antibodies suitable for practicing the methods of the invention are preferably monoclonal, and may be multispecific, human, humanized or chimeric antibodies, single chain antibodies, Fab fragments, F(ab′) fragments, fragments produced by a Fab expression library, and CD30 binding fragments of any of the above. The term “antibody,” as used herein, refers to immunoglobulin molecules and immunologically active portions of immunoglobulin molecules, i.e., molecules that contain an antigen binding site that immunospecifically binds CD30. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the invention, the antibodies are human antigen-binding antibody fragments of the present invention and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding antibody fragments, including single-chain antibodies, may comprise the variable region(s) alone or in combination with the entirety or a portion of the following: hinge region, CH1, CH2, CH3 and CL domains. Also included in the invention are antigen-binding fragments also comprising any combination of variable region(s) with a hinge region, CH1, CH2, CH3 and CL domains. Preferably, the antibodies are human, murine (e.g., mouse and rat), donkey, sheep, rabbit, goal, guinea pig, camelid, horse, or chicken. As used herein, ‘human ’ antibodies include antibodies having the amino acid sequence of a human immunoglobulin and include antibodies isolated from human immunoglobulin libraries, from human B cells, or from animals transgenic for one or more human immunoglobulin, as described infra and, for example in U.S. Pat. No. 5,939,598 by Kucherlapati et al.

The antibodies suitable for practicing the methods of the present invention may be monospecific, bispecific, trispecific or of greater multispecificity. Multispecific antibodies may be specific for different epitopes of CD30 or may be specific for both CD30 as well as for a heterologous protein. See, e.g., PCT publications WO 93/17715; WO 92/08802; WO 91/00360; WO 92/05793; Tutt, et al., 1991, J. Immunol. 147:60-69; U.S. Pat. Nos. 4,474,893; 4,714,681; 4,925,648; 5,573,920; 5,601,819; Kostelny et al., 1992, J. Immunol. 148:1547-1553. Multispecific antibodies, including bispecific and trispecific antibodies, useful for practicing the present invention are antibodies that immunospecifically bind to both CD30 (including but not limited to antibodies that have the CDRs and/or heavy chains of the monoclonal antibodies Ki-2, Ki-4, Ki-5, Ki-7, Ber-H2, HRS-1, HRS-4, Ki-1, Ki-6, M67, Ki-3, M44, HeFi-1, and AC10) and a lymphocyte surface receptor or receptor complex, such as an immunoglobulin gene superfamily member, a TNF receptor superfamily member, an integrin, a cytokine receptor, a chemokine receptor, a major histocompatibility protein, a lectin (C-type, S-type, or I-type), or a complement control protein. Examples of such molecules, and antibodies against such molecules from which a bispecific antibody can be derived, are provided in Section 5.12.2, infra. In a preferred embodiment, the binding of the portion of the multispecific antibody to the lymphocyte cell surface molecule or molecular complex enhances the cytotoxic or cytostatic effect of the anti-CD30 antibody by delivering a cytostatic or cytotoxic signal to the activated lymphocytes.

In certain specific embodiment of the present invention, the anti-CD30 antibody is an agonistic antibody. In another specific embodiment of the present invention, the anti-CD30 antibody is not a non-agonistic antibody. In another specific embodiment, the anti-CD30 antibody does not block binding of CD30 ligand to CD30.

Antibodies useful in the present methods may be described or specified in terms of the particular CDRs they comprise. In certain embodiments antibodies of the invention comprise one or more CDRs of AC10 and/or HeFi-1. The invention encompasses the use of an antibody or derivative thereof comprising a heavy or light chain variable domain, said variable domain comprising (a) a set of three CDRs, in which said set of CDRs are from monoclonal antibody AC10 or HeFi-1, and (b) a set of four framework regions, in which said set of framework regions differs from the set of framework regions in monoclonal antibody AC10 or HeFi-1, respectively, and in which said antibody or derivative thereof immunospecifically binds CD30 and induces CD30 signaling in a lymphocyte and/or exerts a cytotoxic or cytostatic effect on activated lymphocytes.

In a specific embodiment, the invention encompasses the use of an antibody or derivative thereof for treating an immunological disorder, wherein the antibody comprises a heavy chain variable domain, said variable domain comprising (a) a set of three CDRs, in which said set of CDRs comprises SEQ ID NO:4, 6, or 8 and (b) a set of four framework regions, in which said set of framework regions differs from the set of framework regions in monoclonal antibody AC10, and in which said antibody or derivative thereof immunospecifically binds CD30, and induces CD30 signaling in a lymphocyte and/or exerts a cytotoxic or cytostatic effect on activated lymphocytes.

In a specific embodiment, the invention encompasses the use of an antibody or derivative thereof for the treatment of an immunological disorder, wherein the antibody comprises a heavy chain variable domain, said variable domain comprising (a) a set of three CDRs, in which said set of CDRs comprises SEQ ID NO:20, 22 or 24 and (b) a set of four framework regions, in which said set of framework regions differs from the set of framework regions in monoclonal antibody HeFi-1, and in which said antibody or derivative thereof immunospecifically binds CD30, and induces CD30 signaling in a lymphocyte and/or exerts a cytotoxic or cytostatic effect on activated lymphocytes.

In a specific embodiment, the invention encompasses the use of an antibody or derivative thereof for the treatment of an immunological disorder, wherein the antibody comprises a light chain variable domain, said variable domain comprising (a) a set of three CDRs, in which said set of CDRs comprises SEQ ID NO: 12, 14 or 16, and (b) a set of four framework regions, in which said set of framework regions differs from the set of framework regions in monoclonal antibody AC10, and in which said antibody or derivative thereof immunospecifically binds CD30, and induces CD30 signaling, in a lymphocyte and/or exerts a cytotoxic or cytostatic effect on activated lymphocytes.

In a specific embodiment, the invention encompasses using an antibody or derivative thereof for the treatment of an immunological disease, wherein the antibody comprises a light chain variable domain, said variable domain comprising (a) a set of three CDRs, in which said set of CDRs comprises SEQ ID NO:28, 30, or 32, and (b) a set of four framework regions, in which said set of framework regions differs from the set of framework regions in monoclonal antibody HeFi-1, and in which said antibody or derivative thereof immunospecifically binds CD30, and induces CD30 signaling in a lymphocyte and/or exerts a cytotoxic or cytostatic effect on activated lymphocytes.

Additionally, antibodies that may be used in the methods of the present invention may also be described or specified in terms of their primary structures. Antibodies having at least 50%, at least 55%, at least 60%, at least 65%, at least 70%, at least 75%, at least 80%, at least 85%, at least 90%, at least 95% and most preferably at least 98% identity (as calculated using methods known in the art and described herein) to the variable regions and AC10 or HeFi-1 are also included in the present methods for treating immunological disorders. Antibodies useful in the methods of the present invention may also be described or specified in terms of their binding affinity to CD30. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×⁻¹³ M, 10⁻¹³ M, 5×10⁻¹⁴ M, 10⁻¹⁴ M, 5×10⁻¹⁵ M, or 10⁻¹⁵ M.

The antibodies of the invention, i.e., antibodies that are useful for treating immunological disorders, include derivatives that are modified, i.e., by the covalent attachment of any type of molecule to the antibody such that covalent attachment does not prevent the antibody from binding to CD30 or from exerting a cytostatic or cytotoxic effect on activated lymphocytes. For example, but not by way of limitation, the antibody derivatives include antibodies that have been modified, e.g., by glycosylation, acetylation, pegylation, phosphorylation, amidation, derivatization by known protecting/blocking groups, proteolytic),tic cleavage, linkage to a cellular ligand or other protein, etc. Any of numerous chemical modifications may be carried out by known techniques, including, but not limited to specific chemical cleavage, acetylation, formylation, metabolic synthesis of turicamycin, etc. Additionally, the derivative may contain one or more non-classical amino acids.

The antibodies that may be used in the treatment of immunological disorders may be generated by any suitable method known in the art. Polyclonal antibodies to CD30 can be produced by various procedures well known in the art. For example, CD30 can be administered to various host animals including, but not limited to, rabbits, mice, rats, etc. to induce the production of sera containing polyclonal antibodies specific for the protein. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed., 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and resell known in the art. In a non-limiting example, mice can be immunized with CD30 or a cell expressing CD30 or a fragment or derivative thereof. Once an immune response is detected, e.g. antibodies specific for CD3′ are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding CD30 and exerting a cytotoxic or cytostatic effect on activated lymphocytes. Ascites fluid, which generally contains high levels of antibodies, can be generated by injecting mice with positive hybridoma clones.

Antibody fragments which recognize specific epitopes may be generated by known techniques. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH 1 domain of the heavy chain.

For example, antibodies useful in the methods of the present invention can also be generated using various phage display methods known in the art. In phage display methods, functional antibody domains are displayed on the surface of phage particles which carry the nucleic acid sequences encoding them. In a particular embodiment, such phage can be utilized to display antigen binding domains expressed from a repertoire or combinatorial antibody library (e.g. human or murine). In phage display methods, functional antibody domains are displayed on the surface of phage particles which carry the nucleic acid sequences encoding them. In particular, DNA sequences encoding V_{H} and V_{L} domains are amplified from animal cDNA libraries (e.g., human or murine cDNA libraries of lymphoid tissues). The DNA encoding the V_{H} and V_{L} domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 binding domains expressed from phage with Fab, Fv or disulfide stabilized Fv antibody domains recombinantly fused to either the phage gene III or gene VIII protein. Phage expressing an antigen binding domain that binds to CD30 or an AC10 or HeFi-binding portion thereof can be selected or identified with antigen e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology, 191-280; PCT Application No. PCT/GB91/O1 134; PCT Publications WO 90/02809; WO 91/10737; WO 92/01047; WO 92/18619; WO 93/1 1236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in PCT publication WO 92/22324; Mullinax et al., BioTechniques 1992, 12(6):864-869; and Sawai et al., 1995, AJRI 34:26-34; and Better et al., 1988, Science 240:1041-1043 (said references incorporated by reference in their entireties).

Examples of techniques which can be used to produce single-chain Fvs and antibodies include those described in U.S. Pat. Nos. 4,946,778 and 5,258,498; Huston et al., 1991, Methods in Enzymology 203:46-88; Shu et al., 1993, PNAS 90:7995-7999; and Skerra et al., 1988, Science 240:1038-1040. For some uses, including in vivo use of antibodies in humans and in vitro proliferation or cytotoxicity assays, it is preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science, 1985, 229:1202; Oi et al., 1986, BioTechniques 4:214; Gillies et al., 1989, J. Immunol. Methods 125:191-202; U.S. Pat. Nos. 5,807,715; 4,816,567; and 4,816,397, which are incorporated herein by reference in their entirety. Humanized antibodies are antibody, molecules from non-human species antibodies that bind the desired antigen having one or more CDRs from the non-human species and framework and constant regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., 1988, Nature 332:323, which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology, 1991, 28(4/5):489-498; Studnicka et al., 1994, Protein Engineering 7(6):805-814; Roguska. et al., 1994, PNAS 91:969-973), and chain shuffling (U.S. Pat. No. 5,565,332).

Completely human antibodies are particularly desirable for the therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which express human immunoglobulin genes. For example, the human heavy and light chain immunoglobulin gene complexes may be introduced randomly or by homologous recombination into mouse embryonic stem cells. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of CD30. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see, Lonberg and Huszar, 1995, Int. Rev. Immunol. 13:65-93. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.) and Medarex (Princeton, N.J.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., 1994, Bio/technology 12:899-903).

Further, antibodies to CD30 can, in turn, be utilized to generate anti-idiotype antibodies that “mimic” proteins of the invention using techniques well known to those skilled in the art. (See, e.g. Greenspan & Bona, 1989, FASEB J. 7(5):437-444; and Nissinoff. 1991, J. Immunol. 147(8):2429-243S). Fab fragments of such anti-idiotypes can be used in therapeutic regimens to elicit an individual's own immune response against CD30 present on activated lymphocytes.

As alluded to above, proteins that are therapeutically or prophylactically useful against activated lymphocytes need not be antibodies. Accordingly, proteins of the invention may comprise one or more CDRs from an antibody that binds to CD30 and induces CD30 signaling in a lymphocyte and/or exerts a cytotoxic and or cytostatic effect on activated lymphocytes. Preferably, a protein of the invention is a multimer, most preferably a dimer. As used herein, a “protein of the invention” is a protein, including but not limited to an antibody, that binds to CD30, and induces CD30 signaling in a lymphocyte and/or exerts a cytotoxic or cytostatic effect on activated lymphocytes.

The invention also provides methods of treating immunological disorders using proteins, including but not limited to antibodies, that competitively inhibit binding of AC10 or HeFi-1 to CD30 as determined by any method known in the art for determining competitive binding, for example, the immunoassays described herein. In preferred embodiments, the protein competitively inhibits binding of AC10 or HeFi-1 to CD30 by at least 50%, more preferably at least 60%, yet more preferably at least 70%, and most preferably at least 75%. In other embodiments, the protein competitively inhibits binding of AC10 or HeFi-1 to CD30 by at least 80%, at least 85%, at least 90%, or at least 95%.

As discussed in more detail below, the present invention provides methods of treating immunological disorders using proteins, including antibodies, that bind to activated lymphocytes and exert a cytostatic or cytotoxic effect on the lymphocytes. The proteins can be administered either alone or in combination with other compositions in the prevention or treatment of immunological disorders. The proteins may further be recombinantly fused to a heterologous protein at the N- or C-terminus or chemically conjugated (including covalently and non-covalently conjugations) to cytotoxic agents, proteins or other compositions. For example, antibodies of the present invention may be recombinantly fused or conjugated to molecules useful as chemotherapeutics or toxins, or comprise a radionuclide for use as a radio-therapeutic. See, e.g., PCT publications WO 92/08495; WO 91/14438; WO 89/12624; U.S. Pat. No. 5,314,995; and EP 396,387.

Proteins useful in the present methods may be produced recombinantly b fusing the coding region of one or more of the CDRs of an antibody of the invention in frame with a sequence coding for a heterologous protein. The heterologous protein may provide one or more of the following characteristics: added therapeutic benefits; promote stable expression; provide a means of facilitating high yield recombinant expression; or provide a multimerization domain.

In addition to proteins comprising one or more CDRs of an antibody that binds to CD30, and induces CD30 signaling in a lymphocyte and/or exerts a cytotoxic or cytostatic effect on lymphocytes, proteins that are useful in the therapeutic methods of the invention may be identified using any method suitable for screening for protein-protein interactions. Initially, proteins are identified that bind to CD30, then their ability to exert a cytostatic or cytotoxic effect on activated lymphocytes can be determined. Among the traditional methods which can be employed are “interaction cloning” techniques which entail probing expression libraries with labeled CD30 in a manner similar to the technique of antibody probing of λgt11 libraries, supra) a. By way of example and not limitation, this can be achieved as follows: a cDNA clone encoding CD30 (or an AC10 or HeFi-1 binding domain thereof) is modified at the terminus by inserting the phosphorylation site for the heart muscle kinase (HMK) (Blanar & Rutter, 1992, Science 256:1014-1018). The recombinant protein is expressed in E. coli and purified on a GDP-affinity column to homogeneity (Edery et al., 1988, Gene 74:517-525) and labeled using γ³²P-ATP and bovine heart muscle kinase (Sigma) to a specific activity of 1×10⁸ cpm/μg, and used to screen a human placenta λgt11 cDNA library in a “far-Western assay” (Blanar & Rutter, 1992, Science 256:1014-1018). Plaques which interact with the CD30 probe are isolated. The cDNA inserts of positive λ plaques are released and subcloned into a vector suitable for sequencing, such as pBluescript KS (Stratagene).

One method which detects protein interactions is in vivo, the two-hybrid system, is described in detail for illustration purposes only and not by way of limitation. One version of this system has been described (Chien et al., 1991, Proc. Natl. Acad. Sci. USA, 88:9578-9582) and is commercially available from Clontech (Palo Alto, Calif.).

Once a CD30-binding protein is identified, its ability (alone or when multimerized or fused to a dimerization or multimerization domain) to elicit a cytostatic or cytotoxic effect on activated lymphocytes is determined by the methods described in Section 5.7, infra.

Without limitation as to mechanism of action, a protein that binds to CD30 and induces CD30 signaling in a lymphocyte and/or exerts a cytostatic or cytotoxic effect on activated lymphocytes preferably has more than one CD30-binding site and therefore a capacity to cross link CD30 molecules on the surface of an activity ated lymphocyte. Proteins which bind to CD30 or compete for binding to CD30 with AC10 or HeFi-1 can acquire the ability, to induce cytostatic or cytotoxic effects on activated lymphocytes if dimerized or multimerized. Where the CD30-binding protein is a monomeric protein, it can be expressed in tandem, thereby resulting in a protein with multiple CD30 binding sites. The CD30-binding sites can be separated by a flexible linker region. In another embodiment, the CD30-binding proteins can be chemically cross-linked, for example using gluteraldehyde, prior to administration. In a preferred embodiment, the CD30-binding region is fused with a heterologous protein, wherein the heterologous protein comprises a dimerization and multimerization domain. Prior to administration of the protein of the invention to a subject for the purpose of treating or preventing immunological disorders, such a protein is subjected to conditions that allows formation of a homodimer or heterodimer. A heterodimer, as used herein, may comprise identical dimerization domains but different CD30-binding regions, identical CD30-binding regions but different dimerization domains, or different CD30-binding regions and dimerization domains.

Particularly preferred dimerization domains are those that originate from transcription factors.

In one embodiment, the dimerization domain is that of a basic region leucine zipper (“bZIP”). bZIP proteins characteristically possess two domains—a leucine zipper structural domain and a basic domain that is rich in basic amino acids, separated by a “fork” domain (C. Vinson et al., 1989, Science, 246:911-916). Two bZIP proteins dimerize by forming a coiled coil region in which the leucine zipper domains dimerize. Accordingly, these coiled coil regions may be used as fusion partners for proteins that will be useful in the therapeutic methods described herein.

Particularly useful leucine zipper domain are those of the yeast transcription factor GCN4, the mammalian transcription factor CCAAT/enhancer-binding protein C/EBP, and the nuclear transform in oncogene products, Fos and Jun (see Landschultz et al., 1988, Science 240:1759-1764; Baxevanis and Vinson, 1993, Curr. Op. Gen. Devel., 3:278-285; and O'Shea et al., 1989, Science, 243:538-542).

In another embodiment, the dimerization domain is that of a basic-region helix-loop-helix (“bHLH”) protein (Murre et al., 1989, Cell, 56:777-783). bHLH proteins are also composed of discrete domains, the structure of which allows them to recognize and interact with specific sequences of DNA. The helix-loop-helix region promotes dimerization through its amphipathic helices in a fashion analogous to that of the leucine zipper region of the bZIP proteins (Davis et al., 1990 Cell, 60:733-746; Voronova and Baltimore, 1990 Proc. Natl. Acad. Sci. USA, 87:4722-4726). Particularly useful hHLH proteins are myc, max, and mac.

Heterodimers are known to form between Fos and Jun (Bohmann et al., 1987, Science, 238:1386-1392), among members of the ATF/CREB family (Hai et al., 1989, Genes Dev., 3:2083-2090), among members of the C/EBP family (Cao et al., 1991, Genes Dev., 5:1538-1552; Williams et al, 1991, Genes Dev., 5:1553-1567; and Roman et al., 1990, Genes Dev., 4:1404-1415), and between members of the ATF/CREB and Fos/Jun families Hai and Curran, 1991, Proc. Natl. Acad. Sci. USA, 88:3720-3724). Therefore, when a protein of the invention is administered to a subject as a heterodimer comprising different dimerization domains, any combination of the foregoing may be used.

In a preferred aspect, a proteins of the invention, including but not limited to an antibody-drug conjugate of the invention, is substantially purified (e.g., substantially free from substances that limit its effect or produce undesired side-effects). In certain specific embodiments, the protein of the invention is 40% pure, more preferably about 50% pure, and most preferably about 60% pure. In certain specific embodiments, the protein of the invention is approximately 60-65%, 65-70%, 70-75%, 75-80%, 80-85%, 85-90%, 90-95%, or 95-98% pure. In another specific embodiment, the protein of the invention is approximately 99% pure.

5.1.1 Isolation of AC10 or HeFi-1 Genes

The invention relates to the use of AC10 or HeFi-1 nucleic acids, e.g., for gene therapy of immunological disorders or for recombinant expression of an antibody molecule that can be use for the treatment of an immunological disorder. Accordingly, the invention provides purified nucleic acids consisting of at least 8 nucleotides (i.e., a hybridizable portion) of an AC10 or HeFi-1 gene sequence in other embodiments, the nucleic acids consist of at least 25 (contiguous) nucleotides, 50 nucleotides, 100, or 200 nucleotides of an AC10 or HeFi-1 sequence, or a full-length AC10 or HeFi-1 variable region coding sequence. In the same or other embodiments, the nucleic acids are smaller than 50, 75, 100, or 200 or 5000 nucleotides in length. Nucleic acids can be single or double stranded. The invention also relates to nucleic acids hybridizable to or complementary to the foregoing sequences or their reverse complements, and in particular, such nucleic acids that encode proteins that bind to CD30, compete with AC10 or HeFi-1 for binding to CD30, and/or increase the binding of CD30 ligand to CD30 by at least 45%, 50%, 60%, or 65%. In specific aspects, nucleic acids are provided which comprise a sequence complementary to at least 10, 25, 50, 100, or 200 nucleotides or the entire coding region of an AC10 or HeFi-1 variable region gene.

Nucleic acids encoding derivatives and analogs of AC10 or HeFi-1 proteins are additionally provided.

5.1.2 Cloning Procedures

Specific embodiments for the cloning of an AC10 or HeFi-1 nucleic acid follow. In a specific embodiment, total RNA is isolated from a mAb AC10 or HeFi-1-producing hybridoma and polymerase chain reaction is used to amplify desired variable region sequences, using primers based on the sequences disclosed herein. By way of another example, mRNA is isolated from a mAb AC10 or HeFi-1-producing hybridoma, cDNA is made and ligated into an expression vector (e.g., a bacteriophage derivative) such that it is capable of being expressed by the host cell into which it is then introduced. Various screening assays can then be used to select for the expressed product. In one embodiment, selection is on the basis of hybridization to a labeled probe representing a portion of an AC10 or HeFi-1 gene or its RNA or a fragment thereof (Benton and Davis, 1977, Science 196:180; Grunstein and Hogness, 1975, Proc. Natl. Acad. Sci. U.S.A. 72:3961). Those DNA fragments with substantial homology to the probe will hybridize. It is also possible to identify the appropriate fragment by restriction enzyme digestion(s) and comparison of fragment sizes with those expected according to a known restriction map if such is available. Further selection can be carried out on the basis of the properties of the gene.

Alternatively, the presence of the desired gene may be detected by assays based on the physical, chemical, or immunological properties of its expressed product. For example, cDNA clones, or DNA clones which hybrid-select the proper mRNAs, can be selected and expressed to produce a protein that has, e.g., similar or identical electrophoretic migration, isoelectric focusing behavior, proteolytic digestion maps, or functional activity, as known for an AC10 or HeFi-1 protein. For example, ability to bind CD30 can be detected in an ELISA (enzyme-linked immunosorbent assay)-type procedure.

An AC10 or HeFi-1 gene can also be identified by mRNA selection using nucleic acid hybridization followed by in vitro translation. In this procedure, fragments are used to isolate complementary mRNAs by hybridization. Functional assays (e.g., binding to CD30, etc.) of the in vitro translation products of the isolated products of the isolated mRNAs identifies the mRNA and, therefore, the complementary DNA fragments that contain the desired sequences.

In another embodiment, an AC10 or HeFi-1 nucleic, most preferably an AC10 or HeFi-1 nucleic acid encoding the heavy or light chain variables region or a heavy or light chain CDR, can be chemically synthesized from the sequences disclosed herein. For example, the AC10 and HeFi-1 sequences can be synthesized by standard methods known in the art, e.g., by use of an automated DNA synthesizer (such as those commercially available from Biosearch, Applied Biosystems, etc.). Alternatively, the AC10 or HeFi-1 nucleic acid may be synthesized by a commercially available service, for example by Blue Heron Biotechnology (Bothell, Wash.) or QIAGEN Inc. (Valencia, Calif.). Other methods of isolating AC10 or HeFi-1 genes known to the skilled artisan can be employed.

The isolated AC10 or HeFi-1 nucleic acid (e.g. a nucleic acid encoding AC10 or HeFi-1 or one or more CDRs or variable regions thereof) can then be inserted into an appropriate cloning vector. A large number of vector-host systems known in the art may be used. Possible vectors include, but are not limited to, plasmids or modified viruses, but the vector system must be compatible with the host cell used. Such vectors include, but are not limited to, bacteriophages such as lambda derivatives, or plasmids such as PBR322 or pUC plasmid derivatives or the Bluescript vector (Stratagene). The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and an AC10 or HeFi-1 gene may be modified by homopolymeric tailing, or by PCR with primers containing the appropriate sequences. Recombinant molecules can be introduced into host cells via transformation, transfection, infection, electroporation, etc., so that many copies of the gene sequence are generated. Antibodies comprising one or more CDRs from AC10 or HeFi-1 and framework regions from a different immunoglobulin molecule can be produced using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, 1991, Molecular Immunology 28(4/5):489-498; Studnicka et al., 1.994, Protein Engineering 7(6):805-814; Roguska et al., 1994, PNAS 91:969-973), and chain shuffling (U.S. Pat. No. 5,565,332).

In a preferred embodiment of the present invention, an AC10 or HeFi-1 nucleic acid, for example a nucleic acid encoding an AC10 or HeFi-1 heavy or light chain variable region, can be cloned into an immunoglobulin expression vector. Briefly, the AC10 or HeFi-1 nucleic acid is synthesized or otherwise obtained by any of the methods described herein, preferably flanked by appropriate restriction sites, then cloned into a vector suitable for expression of immunoglobulin molecules. A number of vectors have been described that contain, for example, sequences encoding immunoglobulin constant regions and restriction sites suitable for in frame cloning of antibody variable regions operably linked to a promoter and optionally a signal sequence useful for expression in a desired host cell. Non-limiting examples of vectors that have been designed for this purpose are described in McLean et al., 2000, Mol Immunol. 37(14):837-45; Liang et al., 2001, J. Immunol Methods 247(1-2):119-30; Persic et al., 1997, Gene 187(1):9-18; Skerra. 1994 Gene 141(1):79-84; Walls et al., 1993, Nucleic Acids Res 21(12):2921-29; Coloma et al., 1992, J. Immunol. Methods 152(1):89-104. These vectors or similarly designed vectors can be used for cloning AC10 and HeFi-1 sequences for expression purposes.

In an alternative method, the desired gene may be identified and isolated after insertion into a suitable cloning vector in a “shot gun” approach. Enrichment for the desired gene, for example, by size fractionization, can be done before insertion into the cloning vector.

In specific embodiments, transformation of host cells with recombinant DNA molecules that incorporate an isolated AC10 or HeFi-1 gene, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The AC10 or HeFi-1 sequences provided by the instant invention include those nucleotide sequences encoding substantially the same amino acid sequences as found in native AC10 or HeFi-1 variable regions, and those encoded amino acid sequences with functionally equivalent amino acids, as well as those encoding other AC10 or HeFi-1 derivatives or analogs, as described below for AC10 or HeFi-1 derivatives and analogs.

5.2 Binding Assays

As described above, the proteins, including antibodies, that are useful for the treatment of immunological disorders according to the methods of the present-invention bind to CD30 and exert a cytostatic or cytotoxic effect on activated lymphocytes. Methods of demonstrating the ability, of a protein to bind to CD30 are described herein.

Antibodies may be assayed for immunospecific binding to CD30 by any method known in the art. The immunoassays which can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et. al., eds., 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody to immunoprecipitate CD30 can be assessed by, e.g., Western blot analysis. One of skill in the art would be knowledgeable as to the parameters that can be modified to increase the binding of the antibody to CD30 and decrease the background (e.g., pre-clearing the cell lysate with sepharose beads). For further discussion regarding immunoprecipitation protocols see, e.g., Ausubel et al., eds., 1994, Current Protocols in Molecular Biology, Vol. 1, John Wiley & Sons, Inc., New York at 10.16.1.

Western blot analysis generally comprises preparing protein samples, electrophoresis of the protein samples in a polyacrlamide eel (e.g., 8%-20% SDS-PAGE depending on the molecular weight of the antigen), transferring the protein sample from the polyacrylamide gel to a membrane such as nitrocellulose. PVDF or nylon, incubating the membrane in blocking solution (e.g., PBS with 3% BSA or non-fat milk), washing the membrane in washing buffer (e.g., PBS-Tween 20), blotting the membrane with primary antibody (i.e., the putative anti-CD30 antibody) diluted in blocking buffer, washing the membrane in washing buffer, incubating the membrane with a secondary antibody (which recognizes the primary antibody, e.g., an anti-human antibody) conjugated to an enzyme substrate (e.g., horseradish peroxidase or alkaline phosphatase) or radioactive molecule (e.g., ³²P or ¹²⁵I) diluted in blocking buffer, washing the membrane in wash buffer, and detecting the presence of the secondary antibody. One of skill in the art would be knowledgeable as to the parameters that can be modified to increase the signal detected and to reduce the background noise. For further discussion regarding Western blot protocols see, e.g., Ausubel et al., eds., 1994, Current Protocols in Molecular Biology, Vol. 1, John Wiley & Sons, Inc., New York at 10.8.1.

ELISAs comprise preparing antigen (i.e., CD30), coating the well of a 96 well microtiter plate with the CD30, adding the antibody conjugated to a detectable compound such as an enzyme (e.g., horseradish peroxidase or alkaline phosphatase) to the well and incubating for a period of time, and detecting the presence of the antibody. In ELISAs the antibody does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of CD30 protein to the coated well. One of skill in the art would be knowledgeable as to the parameters that can be modified to increase the signal detected as well as other variations of ELISAs known in the art. For further discussion regarding ELISAs see, e.g., Ausubel et al. eds., 1994, Current Protocols in Molecular Biology, Vol. 1, John Wiley & Sons, Inc., New York at 11.2.1.

The binding affinity of an antibody to CD30 and the off-rate of an antibody CD30 interaction can be determined by competitive binding assays. One example of a competitive binding assay is a radioimmunoassay comprising the incubation of labeled CD30 (e.g., ³H or ¹²⁵I) with the antibody of interest in the presence of increasing amounts of unlabeled CD30, and the detection of the antibody bound to the labeled CD30. The affinity of the antibody for CD30 and the binding off-rates can then be determined from the data by Scatchard plot analysis. Competition with a second antibody (such as AC10 or HeFi-1) can also be determined using radioimmunoassays. In this case, CD30 is incubated with the antibody of interest conjugated to a labeled compound (e.g. ³H or ¹²⁵I) in the presence of increasing amounts of an unlabeled second antibody. Alternatively, the binding affinity of an antibody to CD30 and the on- and off-rates of an antibody-CD30 interaction can be determined by surface plasmon resonance.

Proteins that are useful in the methods of the invention may also be assayed for their ability to bind to CD30 by a standard assay known in the art. Such assays include far Westerns and the yeast two hybrid system. These assays are described in Section 5.2, supra. Another variation on the far Western technique described above entails measuring the ability of a labeled candidate protein to bind to CD30 in a Western blot. In one non-limiting example of a far Western blot, CD30 or the fragment thereof of interest is expressed as a fusion protein further comprising glutathione-S-transferase (GST) and a protein serine/threonine kinase recognition site (such as a cAMP-dependent kinase recognition site). The fusion protein is purified on glutathione-Sepharose beads (Pharmacia Biotech) and labeled with bovine heart kinase (Sigma) and 100 μCi of ³²P-ATP (Amersham). The test protein(s) of interest are separated by SDS-PAGE and blotted to a nitrocellulose membrane, then incubated with the labeled CD30. Thereafter, the membrane is washed and the radioactivity quantitated. Conversely, the protein of interest can be labeled by the same method and used to probe a nitrocellulose membrane onto which CD30 has been blotted.

5.3 Sequences Related to AC10 and HeFi-1

The present invention further encompasses the use of proteins and nucleic acids comprising a region of homology to CDRs of AC10 and HeFi-1, or the coding regions therefor, respectively, for the treatment or prevention of an immunological disorder. In various embodiments, the region of homology is characterized by at least 50%, at least 55%, at least 60%, at least 65%, at least 70%, at least 75%, at least 80%, at least 85%, at least 90%, at least 95% or at least 98% identity with the corresponding region of AC10 or HeFi-1.

In one embodiment, the present invention provides a method of treating or preventing an immunological disorder comprising administering to a patient in need thereof a protein with a region of homology to a CDR of HeFi-1 (SEQ ID NO:20, SEQ ID NO:22′; SEQ ID NO:24; SEQ ID NO:28, SEQ ID NO:30 or SEQ ID NO:32), provided that the protein induces CD30 signaling in a lymphocyte and/or exerts a cytotoxic or cytostatic effect on activated lymphocytes. In another embodiment, the present invention provides a method of treating or preventing an immunological disorder comprising administering to a patient in need thereof a protein with a region of homology to a CDR of AC10 (SEQ ID NO:4; SEQ ID NO:6; SEQ ID NO:8; SEQ ID NO:12; SEQ ID NO:14; or SEQ ID NO:16), provided that the protein induces CD30 signaling in a lymphocyte 90 and/or exerts a cytotoxic or cytostatic effect on activated lymphocytes.

In another embodiment, the present invention provides a method of treating or preventing an immunological disorder comprising administering to a patient in need thereof a nucleic acid with a region of homology to a CDR coding region of HeFi-1 (SEQ ID NO:19, SEQ ID NO:21, SEQ ID NO:23, SEQ ID NO:27, SEQ ID NO:29 or SEQ ID NO:31), provided that the encoded protein induces CD30 signaling in a lymphocyte and/or exerts a cytotoxic or cytostatic effect on activated lymphocytes. In yet another embodiment, the present invention provides a method of treating or preventing an immunological disorder comprising administering to a patient in need thereof a nucleic acid with a region of homology to a CDR coding region of AC10 (SEQ ID NO:3, SEQ ID NO:5. SEQ ID NO:7, SEQ ID NO: 11, SEQ ID NO: 13, SEQ ID NO: 15), provided that the encoded protein induces CD30 signaling in a lymphocyte and/or exerts a cytotoxic or cytostatic effect on activated lymphocytes.

The present invention further encompasses methods of treating or preventing an immunological disorder comprising administering to a patient in need thereof a protein or nucleic acids comprising a region of homology to the variable regions of AC10 and HeFi-1, or the coding region therefor, respectively. In various embodiments, the region of homology is characterized by at least 50%, at least 55%, at least 60%, at least 65%, at least 70%, at least 75%, at least 80%, at least 85%, at least 90%, at least 95% or at least 98% identity with the corresponding region of AC10 or HeFi-1.

In one embodiment, the present invention provides methods of treating or preventing an immunological disorder comprising administering to a patient in need thereof a protein with a region of homology to a variable region of HeFi-1 (SEQ ID NO: 18 or SEQ ID NO: 26). In another embodiment, the present invention provides methods of treating or preventing an immunological disorder comprising administering to a patient in need thereof a protein with a region of homology to a variable region of AC10 (SEQ ID NO: 2 or SEQ ID NO: 10).

In one embodiment, the present invention provides methods of treating or preventing an immunological disorder comprising administering to a patient in need thereof a nucleic acid with a region of homology to a variable region coding region of HeFi-1 (SEQ ID NO:17 or SEQ ID NO:25). In another embodiment, the present invention provides methods of treating or preventing an immunological disorder comprising administering to a patient in need thereof a nucleic with a region of homology to a variable region coding region of AC10 (SEQ ID NO:1 or SEQ ID NO:9).

To determine the percent identity of two amino acid sequences or of two nucleic acids, e.g. between the sequences of an AC10 or HeFi-1 variable region and sequences from other proteins with regions of homology to the AC10 or HeFi-1 variable region, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment, the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al, 1990, J. Mol. Biol. 215:403-410. BLAST nucleotide searches can be performed with the NBLAST program, score=100, wordlength=12 to obtain nucleotide sequences homologous to a nucleic acid encoding a SCA-1 modifier protein. BLAST protein searches can be performed with the XBLAST program, score=50, wordlength=3 to obtain amino acid sequences homologous to a SCA-1 modifier protein. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci., 10:3-5; and FASTA described in Pearson and Lipman, 1988, Proc. Natl. Acad. Sci. 85:2444-8. Within FASTA, ktup is a control option that sets the sensitivity and speed of the search. If ktup=2, similar regions in the two sequences being compared are found by looking at pairs of aligned residues if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see http://bioweb.pasteur.fr/docs/man/man/fasta.1.html#sect2, the contents of which are incorporated herein by reference.

Alternatively, protein sequence alignment may be carried out using the CLUSTAL W algorithm, as described by Higgins et al., 1996, Methods Enzymol. 266:383-402.

The percent identity between two sequences can be determined using techniques similar to those described above, with or without allowing gaps. In calculating percent identity, only exact matches are counted.

5.4 Methods of Producing the Proteins of the Indention

The proteins, including antibodies, that are useful in the methods of the present invention can be produced by any method known in the art for the synthesis of proteins, in particular, by chemical synthesis or preferably, by recombinant expression techniques.

Recombinant expression of a protein that binds to CD30 and induces CD30 signaling in a lymphocyte and/or exerts a cytotoxic or cytostatic effect on activated lymphocytes requires construction of an expression vector containing a nucleic acid that encodes the protein. Once a nucleic acid encoding such a protein has been obtained, the vector for the production of the protein molecule may be produced by recombinant DNA technology using techniques well known in the art. Thus, methods for preparing a protein by expressing a nucleic acid containing nucleotide sequence encoding said protein are described herein. Methods which are well known to those skilled in the art can be used to construct expression vectors containing coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding a protein of the invention operably linked to a promoter. Wherein the protein is an antibody, the nucleotide sequence may encode a heave or light chain thereof, or a heave or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,12′,464) and the variable domain of the antibody may be cloned into such a vector for expression of the entire heavy or light chain.

The expression vector is transferred to a host cell by conventional techniques and the transfected cells are then cultured by conventional techniques to produce a protein of the invention. Thus, the invention encompasses host cells containing a nucleic acid encoding a protein of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the proteins molecules of the invention. Such host-expression systems represent vehicles by which the coding sequences of interest may be produced and subsequently purified, but also represent cells which may, when transformed or transfected with the appropriate nucleotide coding sequences, express a protein of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter). Preferably, bacterial cells such as Escherichia coli, and more preferably, eukaryotic cells, especially for the expression of whole recombinant antibody molecules, are used for the expression of a recombinant protein of the invention. For example, mammalian cells such as Chinese hamster ovary cells (CHO), in conjunction with a vector such as the major intermediate early gene promoter element from human cytomegalovirus is an effective expression system for proteins of the invention (Foecking et al., 1986, Gene 45: 101; Cockett et al., 1990, Bio/Technology 8:2).

In bacterial systems, a number of expression vectors may be advantageously selected depending upon the use intended for the folding and post-translation modification requirements protein being expressed. Where possible, when a large quantity of such a protein is to be produced, for the generation of pharmaceutical compositions comprising a protein of the invention, vectors which direct the expression of high levels of fusion protein products that are readily purified may be desirable. Such vectors include, but are not limited, to the E. coli expression vector pUR278 (Ruther et al., 1983, EMBO 1. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express fusion proteins with glutathione S-transferase (GST). In general, such fusion proteins are soluble and can easily be purified from lysed cells by adsorption and binding to matrix glutathioneagarose beads followed by elution in the presence of free glutatluone. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the coding sequence of the protein of the invention may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the protein of the invention in infected hosts. (See, e.g., Logan & Shenk, 1984, Proc. Natl. Acad. Sci. USA 8 1:355-359). Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein of the invention. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, and W138.

For long-term, high-yield production of recombinant proteins that bind to CD30 and exert a cytotoxic or cytostatic effect on activated lymphocytes, stable expression is preferred. For example, cell lines which stably express the protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the protein of the invention.

A number of selection systems may be used, including but not limited to the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Goldspiel et al., Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932 and Morgan and Anderson, 1993, Ann. Rev. Biochem. 62: 191-217; May, 1993, TIB TECH 11(5):155-215); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics. John Wiley & Sons, NY (1994); Colberre-Garapin et al., 1981, J. Mol. Biol. 150:1, which are incorporated by reference herein in their entireties.

The expression levels of a protein of the invention can be increased by vector amplification (for a review, see Bebbington and Hentschel, “The Use of Vectors Based on Gene Amplification for the Expression of Cloned Genes in Mammalian Cells in DNA Cloning”, Vol.3. (Academic Press, New York. 1987)). When a marker in the vector system expressing antibody is amplifiable, increase in the level of inhibitor present in culture of host cell will increase the number of copies of the marker gene. Since the amplified region is associated with the antibody gene, production of the protein of the invention will also increase (Crouse et al., 1983, Mol. Cell. Biol. 3:257).

Wherein the protein of the invention is an antibody, the host cell may be co-transfected with two expression vectors of the invention, the first vector encoding a heavy chain derived protein and the second vector encoding a light chain derived protein. The two vectors may contain identical selectable markers which enable equal expression of heavy and light chain proteins. Alternatively, a single vector may be used which encodes, and is capable of expressing, both heavy and light chain proteins. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once a protein molecule of the invention has been produced by an animal, chemically synthesized, or recombinantly expressed, it may be purified by any method known in the art for purification of proteins, for example, by chromatography (e.g., ion exchange; affinity, particularly by affinity for the specific antigen, Protein A (for antibody molecules, or affinity for a heterologous fusion partner wherein the protein is a fusion protein; and sizing column chromatography), centrifugation, differential solubility, or by any other standard technique for the purification of proteins.

The present invention encompasses the use of proteins of the invention that are fusion proteins, i.e., proteins that are recombinantly fused or chemically conjugated (including both covalent and non-covalent conjugation) to heterologous proteins (of preferably at least 10, 20, 30, 40, 50, 60, 70, 80, 90 or at least 100 amino acids). The fusion does not necessarily need to be direct, but may occur through linker sequences.

The present invention further includes compositions comprising proteins of the invention fused or conjugated to antibody domains other than the variable regions. For example, the proteins of the invention may be fused or conjugated to an antibody Fc region, or portion thereof. The antibody portion fused to a protein of the invention may comprise the constant region, hinge region. CH 1 domain. CR2 domain, and CH3 domain or any combination of whole domains or portions thereof. The proteins may also be fused or conjugated to the above antibody portions to form multimers. For example, Fc portions fused to the proteins of the invention can form dimers through disulfide bonding between the Fc portions. Higher multimeric forms can be made by fusing the proteins to portions of IgA and IgM. Methods for fusing or conjugating the proteins of the invention to antibody portions are known in the art. See, e.g., U.S. Pat. Nos. 5,336,603; 5,622,929; 5,359,046; 5,349,053; 5,447,851; 5,112,946; EP 307,434: EP 367,166; PCT publications WO 96/04388; WO 9 1/06570; Ashkenazi et al. 1991, Proc. Nat. Acad. Sci. USA 88:10535-10539; Zheng et al., 1995, J. Immunol. 154:5590-5600; and Vil et al., 1992, Proc. Natl. Acad. Sci. USA 89:11337-11341 (said references incorporated by reference in their entireties).

5.5 Conjugates and Fusion Proteins

As discussed, supra, the methods of the invention for treatment and prevention of immunological disorders encompass the use of proteins that bind to CD30 and exert a cytostatic and/or cytotoxic effect on activated lymphocytes, and that are further fused or conjugated to heterologous proteins or cytotoxic agents.

The present invention thus provides for methods of treatment or prevention of immunological disorders by administration of a protein or nucleic acid of the invention. In certain embodiments of the invention, a protein or nucleic acid of the invention may be chemically modified to improve its cytotoxic and/or cytostatic properties. For example, a protein of the invention can be administered as a conjugate. Particularly suitable moieties for conjugation to proteins of the invention are chemotherapeutic agents, pro-drug converting enzymes, radioactive isotopes or compounds, or toxins.

In one embodiment, a protein of the invention is fused to a marker sequence, such as a peptide, to facilitate purification. In preferred embodiments, the marker amino acid sequence is a hexa-histidine peptide, such as the tag provided in a pQE vector (QIAGEN, Inc., 9259 Eton Avenue. Chatsworth, Calif., 91311), among others, many of which are commercially available. As described in Gentz et al. 1989, Proc. Natl. Acad. Sci. USA 86:821-824, for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., 1984, Cell 37:767) and the “flag” tag. Such fusion proteins can be generated by standard recombinant methods known to those of skill in the art.

In another embodiment, the proteins of the invention are fused or conjugated to a therapeutic agent. For example, a protein of the invention may be conjugated to a cytotoxic agent such as a chemotherapeutic agent (see infra Section 5.6), a toxin (e.g. a cytostatic or cytocidal agent), or a radionuclide (e.g., alpha-emitters such as, for example, ²¹²Bi, ²¹¹At, or beta-emitters such as, for example, ¹³¹I, ⁹⁰Y, or ⁶⁷Cu). Examples of additional agents that are useful for conjugating to the anti-CD30 molecules of the invention are provided in Section 5.12.1, infra.

The conjugates of the invention used for enhancing the therapeutic effect of the anti-CD30 antibodies that are useful in the methods of the present invention include non-classical therapeutic agents such as toxins. Such toxins include, but are not limited to, abrin, ricin A, pseudomonas exotoxin, or diphtheria toxin.

Alternatively, an antibody of the invention can be conjugated to a second antibody to form an antibody heteroconjugate as described by Segal in U.S. Pat. No. 4,676,980, which is incorporated herein by reference in its entirety. Heteroconjugates useful for practicing the present invention comprise antibodies or antibody portions that bind to CD30 (including but not limited to antibodies that have the CDRs and/or heavy chains of the monoclonal antibodies Ki-2, Ki-4, Ki-5, Ki-7, Ber-H2, HRS-1, HRS-4, Ki-1, Ki-6, M67, Ki-3, M44, HeFi-1, and AC10) and antibody or a antibody portions that bind to a lymphocyte surface receptor or receptor complex, such as an immunoglobulin gene superfamily member, a TNF receptor superfamily member, an integrin, a cytokine receptor, a chemokine receptor, a major histocompatibility protein, a lectin (C-type, S-type, or I-type), or a complement control protein. Examples of such molecules, and antibodies against such molecules that can be used to make heteroconjugates, are provided in Section 5.12.2, infra.

As discussed above, in certain embodiments of the invention, a protein of the invention can be co-administered with a pro-drug converting enzyme. The pro-drug converting enzyme can be expressed as a fusion protein with or conjugated to a protein of the invention. Exemplary pro-drug converting enzymes are carboxypeptidase G2, beta-glucuronidase, penicillin-V-amidase, penicillin-G-amidase, β-lactamase, β-glucosidase, nitroreductase and carboxypeptidase A.

5.6 Antibody-Drug Conjugates

The present invention encompasses the use of anti-CD30 antibody-drug conjugates (anti-CD30 ADCs) for the treatment or prevention of an immunological disorder. The ADCs of the invention are tailored to produce clinically beneficial cytotoxic or cytostatic effects on CD30-expressing cells when administered to a patient with an immune disorder involving CD30-expressing cells, preferably when administered alone but also in combination with other therapeutic agents.

Techniques for conjugating such drugs to proteins, and in particular to antibodies, are well known, see, e.g., Arnon et al, “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc., 1985); Hellstrom et al., “Antibodies For Drug Delivery”, in Controlled Drug Delivery (2nd ed.), Robinson et al. (eds.), pp. 623-53 (Marcel Dekker, Inc., 1987); Thorpe, “Antibody Carriers Of Cytotoxic Agents In Cancer Therapy: A Review”, in Monoclonal Antibodies'84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Because in many of the disease states that are encompassed by the treatment methods of the present invention a significant amount of soluble CD30 is shed from the activated lymphocytes, it is preferable when using an anti-CD30 antibody that is conjugated to a drug (e.g. a cytotoxic agent or an immunosuppressive agent) or prodrug converting enzyme that the drug or prodrug converting enzyme is active in the vicinity of the activated lymphocytes rather than any place in the body that soluble CD30 may be found.

Two approaches may be taken to minimize drug activity outside the activated lymphocytes that are targeted by the anti-CD30 antibodies of the invention: first, an antibody that binds to cell membrane but not soluble CD30 may be used, so that the drug, including drug produced by the actions of the prodrug converting enzyme, is concentrated at the cell surface of the activated lymphocyte. A more preferred approach for minimizing the activity of drugs bound to the antibodies of the invention is to conjugate the drugs in a manner that would reduce their activity unless they are hydrolyzed or cleaved off the antibody. Such methods would employ attaching the drug to the antibodies with linkers that are sensitive to the environment at the cell surface of the activated lymphocyte (e.g., the activity of a protease that is present at the cell surface of the activated lymphocyte) or to the environment inside the activated lymphocyte the conjugate encounters when it is taken up by the activated lymphocyte (e.g., in the endosomal or, for example by virtue of pH sensitivity or protease sensitivity, in the lysosomal environment).

In one embodiment, the linker is an acid-labile hydrazone or hydrazide group that is hydrolyzed in the lysosome (see, e.g., U.S. Pat. No. 5,622,929) In alternative embodiments, drugs can be appended to anti-CD30 antibodies through other acid-labile linkers, such as cis-aconitic amides, orthoesters, acetals and ketals (Dubowchik and Walker, 1999, Pharm. Therapeutics 83:67-123; Neville et al., 1989, Biol. Chem. 264:14653-14661). Such linkers are relatively stable under neutral pH conditions, such as those in the blood, but are unstable at below pH 5, the approximate pH of the lysosome.

In other embodiments, drugs are attached to the anti-CD30 antibodies of the invention using peptide spacers that are cleaved by intracellular proteases. Target enzymes include cathepsins B and D and plasmin, all of which are known to hydrolyze dipeptide drug derivatives resulting in the release of active drug inside target cells (Dubowchik and Walker, 1999, Pharm. Therapeutics 83:67-123). The advantage of using intracellular proteolytic drug release is that the drug is highly attenuated when conjugated and the serum stabilities of the conjugates can be extraordinarily high.

In yet other embodiments, the linker is a malonate linker (Johnson et al., 1995. Anticancer Res. 15:1387-93), a maleimidobeiizoyl linker (Lau et al., 1995, Bioorg-Med-Chem. 3(10):1299-1304), or a 3′-N-amide analog (Lau et al, 1995, Bioorg-Med-Chem. 3(103:1305-12).

The drugs used for conjugation to the anti-CD30 antibodies of the present invention can include conventional chemotherapeutics, such as doxorubicin, paclitaxel, melphalan, vinca alkaloids, methotrexate, mitomycin C, etoposide, and others. In addition, potent agents such CC-1065 analogues, calichiamicin, maytansine, analogues of dolastatin 10, rhizoxin, and palytoxin can be linked to the anti-CD30 antibodies using the conditionally stable linkers to form potent immunoconjugates. Examples of other suitable drugs for conjugation to the anti-CD3 0 antibodies of the present invention are provided in Section 5.12.1, infra.

5.6.1 Linkers

As discussed above in Section 5.6, ADCs are generally made by conjugating a drug to an antibody through a linker. Thus, a majority of the ADCs of the present invention, which comprise an anti-CD30 antibody and a high potency drug and/or an internalization-promoting drug, further comprise a linker. Any linker that is known in the art may be used in the ADCs of the present invention, e.g., bifunctional agents (such as dialdehydes or imidoesters) or branched hydrazone linkers (see, e.g., U.S. Pat. No. 5,824,805, which is incorporated by reference herein in its entirety).

In certain, non-limiting, embodiments of the invention, the linker region between the drug moiety and the antibody moiety of the anti-CD30 ADC is cleavable or hydrolyzable under certain conditions, wherein cleavage or hydrolysis of the linker releases the drug moiety from the antibody moiety. Preferably, the linker is sensitive to cleavage or hydrolysis under intracellular conditions.

In a preferred embodiment, the linker region between the drug moiety and the antibody moiety of the anti-CD30 ADC is hydrolyzable if the pH changes by a certain value or exceeds a certain value. In a particularly preferred embodiment of the invention, the linker is hydrolyzable in the milieu of the lysosome, e.g., under acidic conditions (i.e., a pH of around 5-5.5 or less). In other embodiments, the linker is a peptidyl linker that is cleaved by a peptidase or protease enzyme, including but not limited to a lysosomal protease enzyme, a membrane-associated protease, an intracellular protease, or an endosomal protease. Preferably, the linker is at least two amino acids long, more preferably at least three amino acids long. Peptidyl linkers that are cleavable by enzymes that are present in CD30-expressing cancers are preferred. For example, a peptidyl linker that is cleavable by cathepsin-B (e.g., a Gly-Phe-Leu-Gly linker), a thiol-dependent protease that is highly expressed in cancerous tissue, can be used. Other such linkers are described, e.g., in U.S. Pat. No. 6,214,345, which is incorporated by reference in its entirety herein.

In other, non-mutually exclusive embodiments of the invention, the linker by which the anti-CD30 antibody and the drug of an ADC of the invention are conjugated promotes cellular internalization. In certain embodiments, the linker-drug moiety of the ADC promotes cellular internalization. In certain embodiments, the linker is chosen such that the structure of the entire ADC promotes cellular internalization.

In a specific embodiment of the invention, derivatives of valine-citrulline are used as linker (val-cit linker). The synthesis of doxorubicin with the val-cit linker have been previously described (U.S. Pat. No. 6,214,345 to Dubowchik and Firestone, which is incorporated by reference herein in its entirety).

In another specific embodiment, the linker is a phe-lys linker.

In another specific embodiment, the linker is a thioether linker (see, e.g., U.S. Pat. No. 5,622,929 to Willner et al., which is incorporated by reference herein in its entirety).

In yet another specific embodiment, the linker is a hydrazone linker (see, e.g., U.S. Pat. Nos. 5,122,368 to Greenfield et al. and 5,824,805 to King et al., which are incorporated by reference herein in their entireties).

In yet other specific embodiments, the linker is a disulfide linker. A variety of disulfide linkers are known in the art, including but not limited to those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene). SPDB and SMPT (see, e.g., Thorpe et al., 1987, Cancer Res., 47:5924-5931; Wawrzynczak et al., 1987, In Immunoconjugates: Antibody Conjugates in Radioimagery and Therapy of Cancer, ed. C. W. Vogel, Oxford U. Press, pp. 28-55; see also U.S. Pat. No. 4,880,935 to Thorpe et al., which is incorporated by reference herein in its entirety).

A variety of linkers that can be used with the compositions and methods of the present invention are described in U.S. provisional application No. 60/400,403, entitled “Drug Conjugates and their use for treating cancer, an autoimmune disease or an infectious disease”, by Inventors: Peter D. Senter, Svetlana Doronina and Brian E. Toki, submitted on Jul. 31, 2002, which is incorporated by reference in its entirety herein.

In yet other embodiments of the present invention, the linker unit of an anti-CD30 antibody-linker-drug conjugate (anti-CD30 ADC) links the cytotoxic or cytostatic agent (drug unit; -D) and the anti-CD30 antibody unit (-A). As used herein the term anti-CD30 ADC encompasses anti-CD30 antibody drug conjugates with and without a linker unit. The linker unit has the general formula:
-Tₐ-W_{w}—Y_{y}—
wherein:

-T- is a stretcher unit;
a is 0 or 1;
each —W— is independently an amino acid unit;
w is independently an integer ranging from 2 to 12;
—Y— is a spacer unit; and
y is 0, 1 or 2.

5.6.1.1 The Stretcher Unit

The stretcher unit (-T-), when present, links the anti-CD30 antibody unit to an amino acid unit (—W—). Useful functional groups that can be present on an anti-CD30 antibody, either naturally or via chemical manipulation include, but are not limited to, sulfhydryl, amino, hydroxyl, the anomeric hydroxyl group of a carbohydrate, and carboxyl. Preferred functional groups are sulfhydryl and amino. Sulfhydryl groups can be generated by reduction of the intramolecular disulfide bonds of an anti-CD30 antibody. Alternatively, sulfhydryl groups can be generated by reaction of an amino group of a lysine moiety of an anti-CD30 antibody with 2-iminothiolane (Traut's reagent) or other sulfhydryl generating reagents. In specific embodiments, the anti-CD30 antibody is a recombinant antibody and is engineered to carry one or more lysines. In other embodiments, the recombinant anti-CD30 antibody is engineered to carry additional sulfhydryl groups, e.g., additional cysteines.

In certain specific embodiments, the stretcher unit forms a bond with a sulfur atom of the anti-CD30 antibody unit. The sulfur atom can be derived from a sulfhydryl (—SH) group of a reduced anti-CD30 antibody (A). Representative stretcher units of these embodiments are depicted within the square brackets of Formulas (Ia) and (Ib; see infra), wherein A-, —W—, —Y—, -D, w and y are as defined above and R¹ is selected from —C₁-C₁₀ alkylene-, —C₃-C₈ carbocyclo-, —O—(C₁-C₈alkyl)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, C₁-C₁₀alkylene-(C₃-C₈ heterocyclo)-, —(C₃-C₈ heterocyclo)-C₁-C₁₀ alkylene-, —(CH₂CH₂O)ᵣ—, and —(CH₂CH₂O)ᵣ—CH₂—; and r is an integer ranging from 1-10.

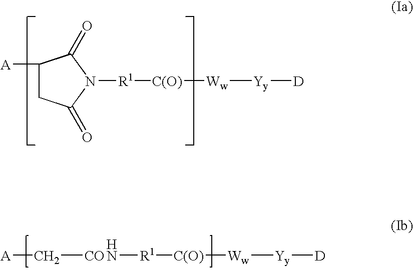

An illustrative stretcher unit is that of formula (Ia) where R¹ is —(CH₂)₅—:

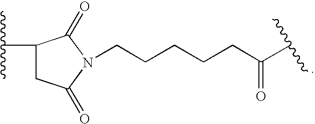

Another illustrative stretcher unit is that of formula (Ia) where R¹ is —(CH₂CH₂O)ᵣ—CH₂—: and r is 2:

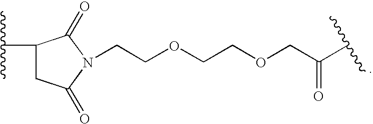

Still another illustrative stretcher unit is that of formula (Ib) where R¹ is —(CH₂)₅—:

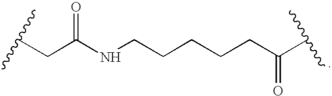

In certain other specific embodiments, the stretcher unit is linked to the anti-CD30 antibody unit (A) via a disulfide bond between a sulfur atom of the anti-CD30 antibody unit and a sulfur atom of the stretcher unit. A representative stretcher unit of this embodiment is depicted within the square brackets of Formula (II), wherein R¹, A-, —W—, —Y—, -D, w and y are as defined above

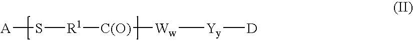

In even other specific embodiments, the reactive group of the stretcher contains a reactive site that can be reactive to an amino group of an anti-CD30 antibody. The amino group can be that of an arginine or a lysine. Suitable amine reactive sites include, but are not limited to, activated esters such as succinimide esters, 4-nitrophenyl esters, pentafluorophenyl esters, anhydrides, acid chlorides, sulfonyl chlorides, isocyanates and isothiocyanates. Representative stretcher units of these embodiments are depicted within the square brackets of Formulas (IIIa) and (IIIb), wherein R¹, A-, —W—, —Y—, -D, w and y are as defined above;

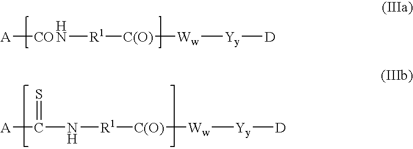

In yet another aspect of the invention, the reactive function of the stretcher contains a reactive site that is reactive to a modified carbohydrate group that can be present on an anti-CD30 antibody. In a specific embodiment, the anti-CD30 antibody is glycosylated enzymatically to provide a carbohydrate moiety. The carbohydrate may be mildly oxidized with a reagent such as sodium periodate and the resulting carbonyl unit of the oxidized carbohydrate can be condensed with a stretcher that contains a functionality such as a hydrazide, an oxime, a reactive amine, a hydrazine, a thiosemicarbazone, a hydrazine carboxylate, and an arylhydrazide such as those described by Kaneko, T. et al. Bioconjugate Chem 1991, 2, 133-41. Representative stretcher units of this embodiment are depicted within the square brackets of Formulas (IVa)-(IVc), wherein R¹, A-, —W—, —Y—, -D, xv and y are as defined above.

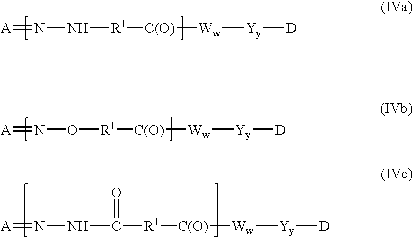

5.6.1.2 The Amino Acid Unit

The amino acid unit (—W—) links the stretcher unit (-T-) to the Spacer unit (—Y—) if the Spacer unit is present, and links the stretcher unit to the cytotoxic or cytostatic agent (Drug unit; D) if the spacer unit is absent.

—W_{w}— is a dipeptide, tripeptide, tetrapeptide, pentapeptide, hexapeptide, heptapeptide, octapeptide, nonapeptide, decapeptide, undecapeptide or dodecapeptide unit. Each —W— unit independently has the formula denoted below in the square brackets, and w is an integer ranging from 2 to 12: 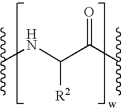
wherein R² is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

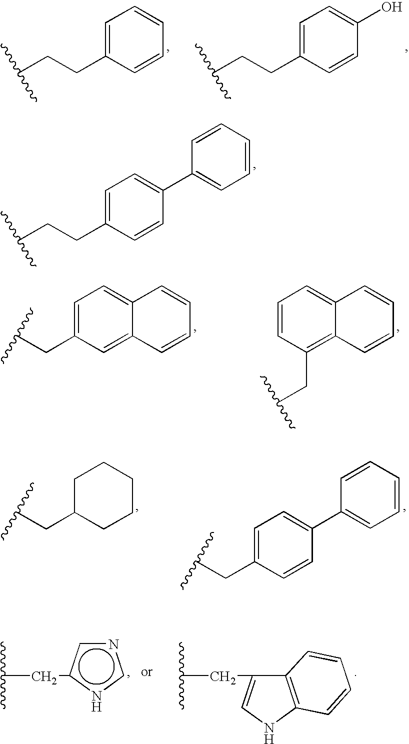

The amino acid unit of the linker unit can be enzymatically cleaved by an enzyme including, but not limited to, a tumor-associated protease to liberate the drug unit (-D) which is protonated in vivo upon release to provide a cytotoxic drug (D).

Illustrative W_{w} units are represented by formulas (V)-(VII):

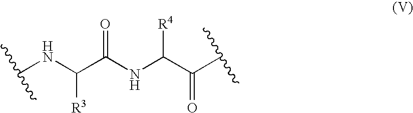

wherein R³ and R⁴ are as follows:

| R³ | R⁴ |
|---|---|
| benzyl | (CH₂)₄NH₂; |
| methyl | (CH₂)₄NH₂; |
| isopropyl | (CH₂)₄NH₂; |
| isopropyl | (CH₂)₃NHCONH₂; |
| benzyl | (CH₂)₃NHCONH₂; |
| isobutyl | (CH₂)₃NHCONH₂; |
| sec-butyl | (CH₂)₃NHCONH₂; |
| 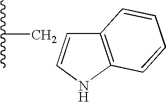 | (CH₂)₃NHCONH₂; |
| benzyl | methyl; and |
| benzyl | (CH₂)₃NHC(═NH)NH₂; |

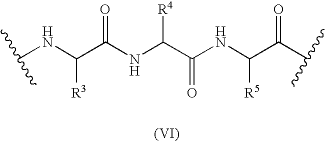

wherein R³, R⁴ and R⁵ are as follows:

| R³ | R⁴ | R⁵ |
|---|---|---|
| benzyl | benzyl | (CH₂)₄NH₂; |
| isopropyl | benzyl | (CH₂)₄NH₂; and |
| H | benzyl | (CH₂)₄NH₂; |

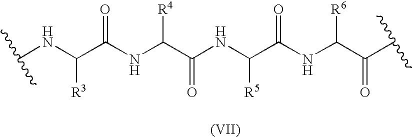

wherein R³, R⁴, R⁵ and R⁶ are as follows:

| R³ | R⁴ | R⁵ | R⁶ |
|---|---|---|---|
| H | benzyl | isobutyl | H; and |
| methyl | isobutyl | methyl | isobutyl. |

Preferred amino acid units include, but are not limited to, units of formula (V) where: R³ is benzyl and R⁴ is —(CH₂)₄NH₂; R³ is isopropyl and R⁴ is —(CH₂)₄NH₂; R³ is isopropyl and R⁴ is —(CH₂)₃NHCONH₂. Another preferred amino acid unit is a unit of formula (VI), where: R³ is benzyl, R⁴ is benzyl, and R⁵ is —(CH₂)₄NH₂.

—W_{w}— units useful in the present invention can be designed and optimized in their selectivity for enzymatic cleavage by a particular tumor-associated protease. The preferred —W_{w}— units are those whose cleavage is catalyzed by the proteases, cathepsin B, C and D, and plasmin.

In one embodiment, —W_{w}— is a dipeptide, tripeptide or tetrapeptide unit.

Where R², R³, R⁴, R⁵ or R⁶ is other than hydrogen, the carbon atom to which R², R³, R⁴, R⁵ or R⁶ is attached is chiral.

Each carbon atom to which R², R³, R⁴, R⁵ or R⁶ is attached is independently in the (S) or (R) configuration.

In a preferred embodiment, the amino acid unit is a phenylalanine-lysine dipeptide (phe-lys or FK linker). In another preferred embodiment, the amino acid unit is a valine-citrulline dipeptide (val-cit or VC linker).

5.6.1.3 The Spacer Unit

The spacer unit (—Y—), when present, links an amino acid unit to the drug unit. Spacer units are of two general types: self-immolative and non self-immolative. A non self-immolative spacer unit is one in which part or all of the spacer unit remains bound to the drug unit after enzymatic cleavage of an amino acid unit from the anti-CD30 antibody-linker-drug conjugate or the drug-linker compound. Examples of a non self-immolative spacer unit include, but are not limited to a (glycine-glycine) spacer unit and a glycine spacer unit (both depicted in Scheme 1). When an anti-CD30 antibody-linker-drug conjugate of the invention containing a glycine-glycine spacer unit or a glycine spacer unit undergoes enzymatic cleavage via a tumor-cell associated-protease, a cancer-cell-associated protease or a lymphocyte-associated protease, a glycine-glycine-drug moiety or a glycine-drug moiety is cleaved from A-T-W_{w}—. To liberate the drug, an independent hydrolysis reaction should take place within the target cell to cleave the glycine-drug unit bond.

In a preferred embodiment, -Y_{y}— is a p-aminobenzyl ether which can be substituted with Qₘ where Q is is —C₁-C₈ alkyl, —C₁-C₈ alkoxy, -halogen, -nitro or -cyano; and m is an integer ranging from 0-4.

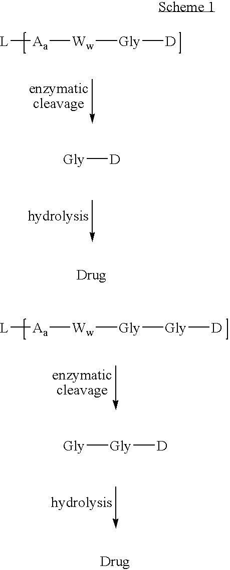

In one embodiment, a non self-immolative spacer unit (—Y—) is -Gly-Gly-.

In another embodiment, a non self-immolative the spacer unit (—Y—) is -Gly-.

In one embodiment, the drug-linker compound or an anti-CD30 antibody-linker-dig conjugate lacks a spacer unit (y=0).

Alternatively, an anti-CD30 antibody-linker-drug conjugate of the invention containing a self-immolative spacer unit can release the drug (D) without the need for a separate hydrolysis step. In these embodiments, —Y— is a p-aminobenzyl alcohol (PAB) unit that is linked to —W_{w}— via the nitrogen atom of the PAB group, and connected directly to -D via a carbonate, carbamate or ether group (Scheme 2 and Scheme 3). 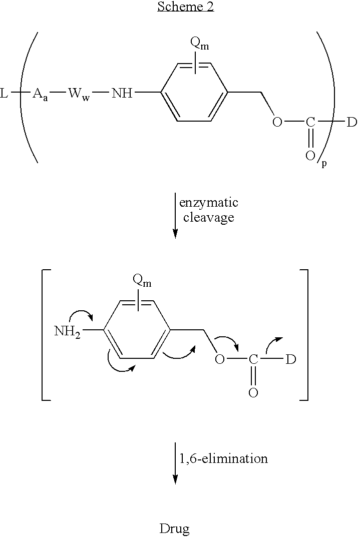
where Q is —C₁-C₈ alkyl, —C₁-C₈ alkoxy, -halogen, -nitro or -cyano; m is an integer ranging from 0-4; and p is an integer ranging from 1-20. 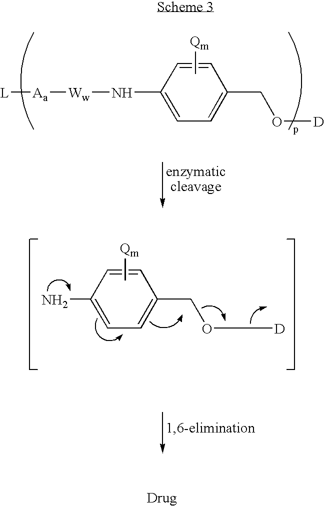
where Q is —C₁-C₈ alkyl, —C₁-C₈ alkoxy, -halogen, -nitro or -cyano; m is an integer ranging from 0-4; and p is an integer ranging from 1-20.

Other examples of self-immolative spacers include, but are not limited to, aromatic compounds that are electronically equivalent to the PAB group such a 2-aminoimidazol-5-methanol derivatives (see Hay et al., Bioorg. Med. Chem. Lett., 1999, 9, 2237 for examples) and ortho or para-aminobenzylacetals. Spacers can be used that undergo facile cyclization upon amide bond hydrolysis, such as substituted and unsubstituted 4-aminobutyric acid amides (Rodrigues et al., Chemistry, Biology, 1995, 2, 223), appropriately substituted bicyclo[2.2.1] and bicyclo[2.2.2] ring systems (Storm, et al., J. Amer. Chem. Soc., 1972, 94, 5815) and 2-aminophenylpropionic acid amides (Amsberry, et al., J. Org. Chem., 1990, 55, 5867). Elimination of amine-containing drugs that are substituted at the α-position of glycine (Kingsbury, et al., J. Med. Chem., 1984, 27, 1447) are also examples of self-immolative spacer strategies that can be applied to the anti-CD30 antibody-linker-drug conjugates of the invention.

In an alternate embodiment, the spacer unit is a branched bis(hydroxymethyl)styrene (BHMS) unit (Scheme 4), which can be used to incorporate additional drugs. 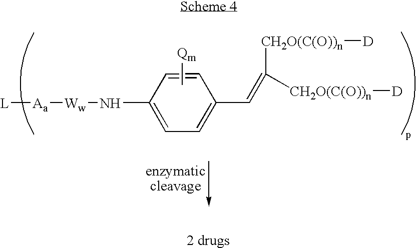
where Q is —C₁-C₈ alkyl, —C₁-C₈ alkoxy, -halogen, -nitro or -cyano; m is an integer ranging from 0-4; n is 0 or 1; and p is an integer raging from 1-20.

In one embodiment, the two -D moieties are the same.

In another embodiment, the two -D moieties are different.

Preferred spacer units (—Y_{y}—) are represented by Formulas (VIII)-(X): 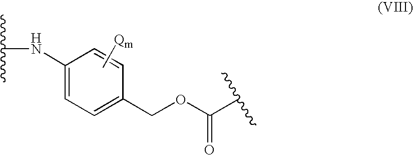
where Q is C₁-C₈ alkyl, C₁-C₈ alkoxy, halogen. nitro or cyano; and m is an integer ranging from 0-4;

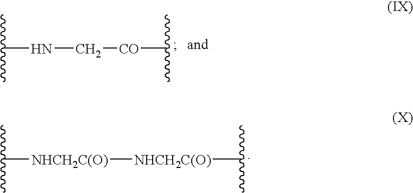

5.6.2 Drugs

The present invention encompasses the use of anti-CD30 ADCs for the treatment or prevention of an immunological disorder. As used herein, the term “drug” or “cytotoxic agent,” where employed in the context of an anti-CD30 ADC of the invention, does not include radioisotopes. Otherwise, any drug that is known to the skilled artisan can be used in connection with the ADCs of the present invention.

The drugs used for conjugation to the anti-CD30 antibodies of the present invention can include conventional chemotherapeutics, such as doxorubicin, paclitaxel, melphalan, vinca alkaloids, methotrexate, mitomycin C, etoposide, and others. In addition, potent agents such CC-1065 analogues, calichiamicin, maytansine, analogues of dolastatin 10, rhizoxin, and palytoxin can be linked to the anti-CD30 antibodies using the conditionally stable linkers to form potent immunoconjugates. Examples of other suitable drugs for conjugation to the anti-CD30 antibodies of the present invention are provided in Section 5.12.1 below.

In certain embodiments, the ADCs of the invention comprise drugs that are at least 40-fold more potent than doxorubicin on CD30-expressing cells. Such drugs include, but are not limited to: DNA minor groove binders, including enediynes and lexitropsins, duocarmycins, taxanes (including paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epithilone A and B, estramustine, cryptophysins, cemadotin, maytansinoids, dolastatins, e.g., auristatin E, dolastatin 10, MMAE, discodermolide, eleutherobin, and mitoxantrone.

In certain specific embodiments, an anti-CD30 ADC of the invention comprises an enediyne moiety. In a specific embodiment, the enediyne moiety is calicheamicin. Enediyne compounds cleave double stranded DNA by generating a diradical via Bergman cyclization.

A variety of cytotoxic and cytostatic agents that can be used with the compositions and methods of the present invention are described in U.S. provisional application No. 60/400,403, entitled “Drug Conjugates and their use for treating cancer, an autoimmune disease or an infectious disease”, by Inventors: Peter D. Senter, Svetlana Doronina and Brian E. Toki, filed on Jul. 31, 2002, which is incorporated by reference in its entirety herein.

In other specific embodiments, the cytotoxic or cytostatic agent is auristatin E or a derivative thereof.

In preferred embodiments, the auristatin E derivative is an ester formed between auristatin E and a keto acid. For example, auristatin E can be reacted with paraacetyl benzoic acid or benzoylvaleric acid to produce AEB and AEVB, respectively. Other preferred auristatin derivatives include MMAE and AEFP.

The synthesis and structure of auristatin E, also known in the art as dolastatin-10, and its derivatives are described in U.S. patent application Ser. Nos. 09/845,786 and 10/001,191; in the International Patent Application No.: PCT/US02/13435, in U.S. Pat. Nos. 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, all of which are incorporated by reference in their entireties herein.

In specific embodiments, the drug is a DNA minor groove binding agent. Examples of such compounds and their syntheses are disclosed in U.S. Pat. No. 6,130,237, which is incorporated by reference in its entirety herein. In certain embodiments, the drug is a CBI compound.

In certain embodiments of the invention, an ADC of the invention comprises an anti-tubulin agent. Anti-tubulin agents are a well established class of cancer therapy compounds. Examples of anti-tubulin agents include, but are not limited to, taxanes (e.g., Taxol® (paclitaxel), docetaxel), T67 (Tularik), vincas, and auristatins (e.g., auristatin E, AEB, AEVB, MMAE, AEFP). Antitubulin agents included in this class are also: vinca alkaloids, including vincristine and vinblastine, vindesine and vinorelbine; taxanes such as paclitaxel and docetaxel and baccatin derivatives, epithilone A and B, nocodazole, colchicine and colcimid, estramustine, cryptophysins, cemadotin, maytansinoids, combretastatins, dolastatins, discodermolide and eleutherobin.

In a specific embodiment, the drug is a maytansinoid, a group of anti-tubulin agents. In a more specific embodiment, the drug is maytansine. Further, in a specific embodiment, the cytotoxic or cytostatic agent is DM-1 (ImmunoGen, Inc.; see also Chari et al. 1992, Cancer Res 52:127-131). Maytansine, a natural product, inhibits tubulin polymerization resulting in a mitotic block and cell death. Thus, the mechanism of action of maytansine appears to be similar to that of vincristine and vinblastine. Maytansine, however, is about 200 to 1,000-fold more cytotoxic in vitro than these vinca alkaloids.

In another specific embodiment, the drug is an AEFP.

In certain specific embodiments of the invention, the drug is not a polypeptide of greater than 50, 100 or 200 amino acids, for example a toxin. In a specific embodiment of the invention, the drug is not ricin.

In other specific embodiments of the invention, an ADC of the invention does not comprise one or more of the cytotoxic or cytostatic agents the following non-mutually exclusive classes of agents: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, nitrosoureas, platinols, purine antimetabolites, puromycins, radiation sensitizers, steroids, taxanes, topoisomerase inhibitors, vinca alkaloids, purine antagonists, and dihydrofolate reductase inhibitors. In more specific embodiments, the high potency drug is not one or more of an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine. VP-16, VM-26, azothioprine, mycophenolate mofetil, methotrexate, acyclovir, gangcyclovir, zidovudine, vidarabine, ribavarin, azidothymidine, cytidine arabinoside, amantadine, dideoxyuridine, iododeoxyuridine, poscarnet, and trifluridine.

5.6.2.1 Dolastatin Drugs

In certain embodiments, the cytotoxic or cytostatic agent is a dolastatin. In more specific embodiments, the dolastatin is of the auristatin class. In a specific embodiment of the invention, the cytotoxic or cytostatic agent is MMAE (MMAE; Formula XI). In another specific embodiment of the invention, the cytotoxic or cytostatic agent is AEFP (Formula XVI).

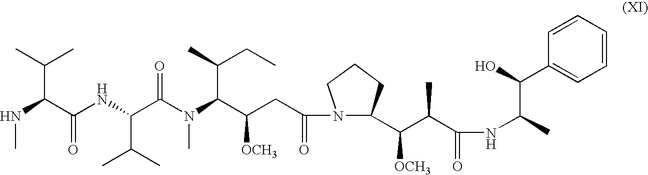

In certain embodiments of the invention, the cytotoxic or cytostatic agent is a dolastatin of formulas XII-XVIII.

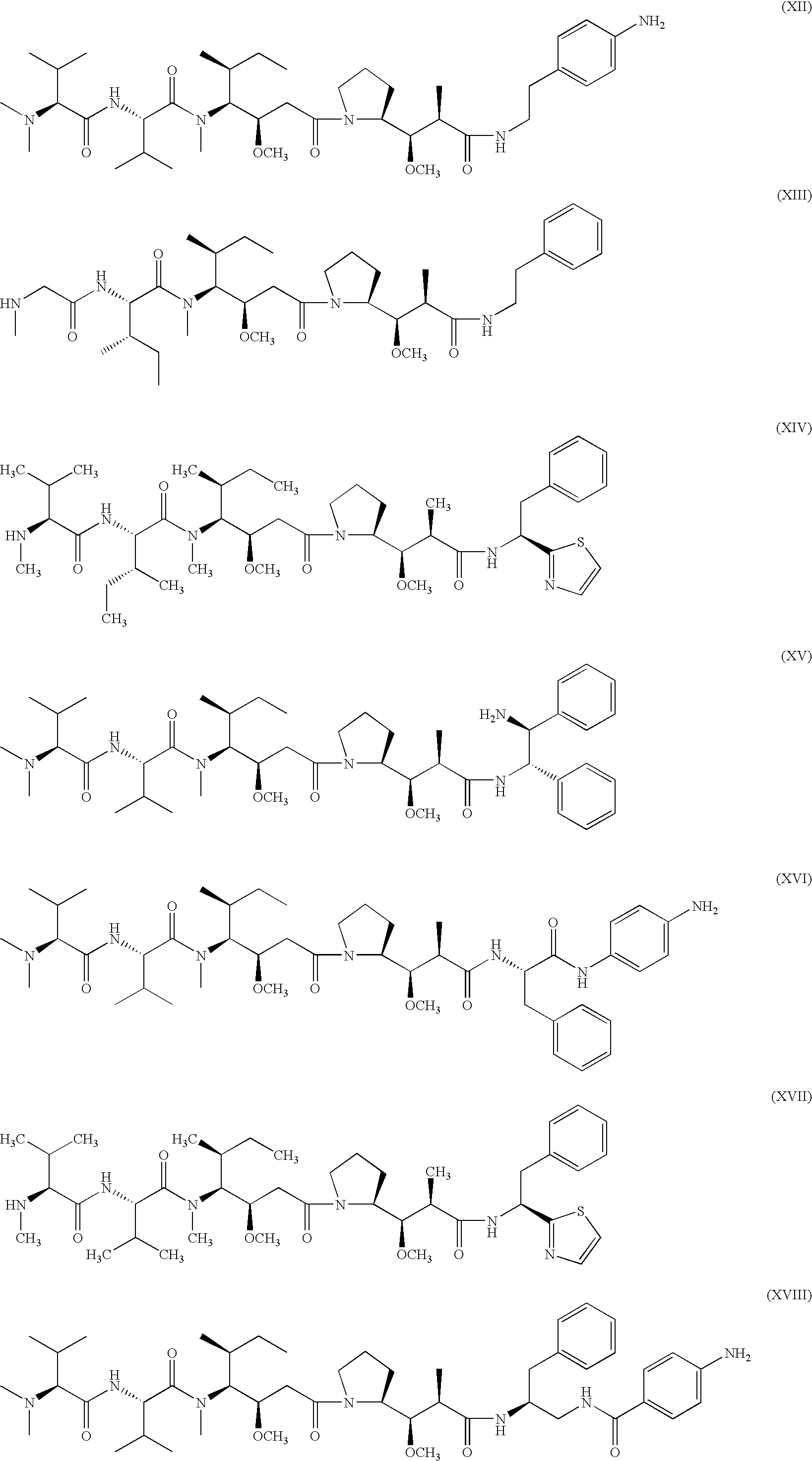

5.6.3 Formation of Anti-CD30 Antibody-Drug Conjugates

The generation of anti-CD30 antibody drug conjugates (ADCs) can be accomplished by any technique known to the skilled artisan. Briefly, the anti-CD30 ADCs comprise an anti-CD30 antibody, a drug, and a linker that joins the drug and the antibody. A number of different reactions are available for covalent attachment of drugs to antibodies. This is often accomplished by reaction of the amino acid residues of the antibody molecule, including the amine groups of lysine, the free carboxylic acid groups of glutamic and aspartic acid, the sulfhydryl groups of cysteine and the various moieties of the aromatic amino acids. One of the most commonly used non-specific methods of covalent attachment is the carbodiimide reaction to link a carboxy (or amino) group of a compound to amino (or carboxy) groups of the antibody. Additionally, bifunctional agents such as dialdehydes or imidoesters have been used to link the amino group of a compound to amino groups of the antibody molecule. Also available for attachment of drugs to antibodies is the Schiff base reaction. This method involves the periodate oxidation of a drug that contains glycol or hydroxy groups, thus forming an aldehyde which is then reacted with the antibody molecule. Attachment occurs via formation of a Schiff base with amino groups of the antibody molecule. Isothiocyanates can also be used as coupling agents for covalently attaching drugs to antibodies. Other techniques known to the skilled artisan and within the scope of the present invention. Non-limiting examples of such techniques are described in, e.g., U.S. Pat. Nos. 5,665,358, 5,643,573, and 5,556,623, which are incorporated by reference in their entireties herein.

In certain embodiments, an intermediate, which is the precursor of the linker, is reacted with the drug under appropriate conditions. In certain embodiments, reactive groups are used on the drug and/or the intermediate. The product of the reaction between the drug and the intermediate, or the derivatized drug, is subsequently reacted with the anti-CD30 antibody under appropriate conditions. Care should be taken to maintain the stability of the antibody under the conditions chosen for the reaction between the derivatized drug and the antibody.

5.7 Assays for Cytotoxic and Cytostatic Activities

By definition, a protein of the invention must exert a cytostatic or cytotoxic effect on an activated lymphocyte. Activated lymphocytes that can be assayed for a cytostatic or cytotoxic effect of a CD30 binding protein may be cultured cell lines (e.g., Jurkat and CESS, both of which are available from the ATCC; or Karpas 299 and L540, both of which are available from Deutsche Sammlung von Mikroorganismen und Zellkulturen GmbH), or from lymphocytes prepared from a fresh blood sample. Lymphocytes can be activated by the appropriate cocktails of antibodies and cytokines, as will be recognized by one of skill in the art. For example, T lymphocytes can be activated using a combination anti-CD3 and anti-CD28 antibodies and IL-2, as described in Section 11 below.

Many methods of determining whether a protein exerts a cytostatic or cytotoxic effect on a cell are known to those of skill in the art, and can be used to elucidate whether a particular protein is a protein of the invention. Illustrative examples of such methods are described below.

Wherein a protein that binds to CD30 does not exert a cytostatic or cytotoxic effect on activated lymphocytes, the protein can be multimerized according to the methods described in Section 5.1, supra, and the multimer assayed for its ability to exert a cytostatic or cytotoxic effect on activated lymphocytes.

In a preferred embodiment, the proteins of the invention are cross-linked prior to assessing their cytotoxic or cytostatic effect on activated lymphocytes. In an exemplary embodiment, in which the protein of the invention is an anti-CD30 antibody, the antibody can be cross-linked in solution, and one or more dilutions of the anti-CD30 antibody can be titrated into 96-well flat bottom tissue culture plates in the absence or presence of secondary antibodies. Activated lymphocytes are then added to the plates at approximately 5,000 cells/well. The cytostatic or cytotoxic effect can then be assessed as described herein, for example as an inhibition of radiolabeled thymidine incorporation into the activated lymphocytes.

Once a protein is identified that both (i) binds to CD30 and (ii) exerts a cytostatic or cytotoxic effect on activated lymphocytes, its therapeutic value is validated in an animal model. Exemplary animal models of immunological disorders are described in Section 5.7.1, infra.

In a preferred embodiment for determining whether an anti-CD30 antibody exerts a cytostatic effect on activated lymphocytes, a thymidine incorporation assay may be used. For example, activated lymphocytes at a density of 5,000 cells/well of a 96-well plated can be cultured for a 72-hour period and exposed to 0.5 μCi of ³H-thymidine during the final 8 hours of the 72-hour period, and the incorporation of ³H-thymidine into cells of the culture is measured in the presence and absence of the antibody.

There are many cytotoxicity assays known to those of skill in the art. Some of these assays measure necrosis, while others measure apoptosis (programmed cell death). Necrosis is accompanied by increased permeability of the plasma membrane; the cells swell and the plasma membrane ruptures within minutes. On the other hand, apoptosis is characterized by membrane blebbing, condensation of cytoplasm and the activation of endogenous endonucleases. Only one of these effects on activated lymphocytes is sufficient to show that a CD30-binding protein is useful in the treatment or prevention of activated lymphocytes as an alternative to the assays measuring cytostatic or cytotoxic effects described above.

In one embodiment, necrosis measured by the ability or inability of a cell to take up a dye such as neutral red, trypan blue, or ALAMAR™ blue (Page et al., 1993, Intl. J. of Oncology 3:473-476). In such an assay, the cells are incubated in media containing the dye, the cells are washed, and the remaining dye, reflecting cellular uptake of the dye, is measured spectrophotometrically.

In another embodiment, the dye is sulforhodamine B (SRB), whose binding to proteins can be used as a measure of cytotoxicity (Skehan et al., 1990, J. Nat'l Cancer Inst. 82:1 107-12).

In yet another embodiment, a tetrazolium salt, such as MTT, is used in a quantitative calorimetric assay for mammalian cell survival and proliferation by detecting living, but not dead, cells (see, e.g., Mosmann, 1983, J. Immunol. Methods 65:55-63).

In yet another embodiment, apoptotic cells are measured in both the attached and “floating” compartments of the cultures. Both compartments are collected by removing the supernatant, trypsinizing the attached cells, and combining both preparations following a centrifugation wash step (10 minutes, 2000 rpm). The protocol for treating tumor cell cultures with sulindac and related compounds to obtain a significant amount of apoptosis has been described in the literature (see, e.g., Piazza et al., 1995, Cancer Research 55:3110-16). Features of this method include collecting both floating and attached cells, identification of the optimal treatment times and dose range for observing apoptosis as detected by DNA fragmentation, and identification of optimal cell culture conditions.

In yet another embodiment, apoptosis is quantitated by measuring DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemical, 1999, no. 2, pp. 34-37 (Roche Molecular Biochemicals).

In yet another embodiment, apoptosis can be observed morphologically. Following treatment with a test protein or nucleic acid, cultures can be assayed for apoptosis and necrosis by fluorescence microscopy following labeling with acridine orange and ethidium bromide. The method for measuring apoptotic cell number has previously been described by Duke & Cohen, 1992, Current Protocols In Immunology, Coligan et al., eds., 3.17.1-3.17.16. In another mode of the embodiment, cells can be labeled with the DNA dye propidium iodide, and the cells observed for morphological changes such as chromatin condensation and margination along the inner nuclear membrane, cytoplasmic condensation, increased membrane blebbing and cellular shrinkage.

5.7.1 Animal Models of Immunological Disorders

The molecules of the invention can be tested or validated in animal models of immunological disorders before they are subjected to clinical testing. A number of established animal models of immunological disorders are known to the skilled artisan, any of which can be used to assay the efficacy of the molecules of the invention. Non-limiting examples of such models are described below.

Some examples for animal models of systemic and organ-specific autoimmune diseases including diabetes, lupus, systemic sclerosis, Sjögren's Syndrome, experimental autoimmune encephalomyelitis (multiple sclerosis), thyroiditis, myasthenia gravis, arthritis, uveitis, inflamatory bowel disease have been described by Bigazzi, P., “Animal Models of Autoimmunity: Spontaneous and Induced”, in The Autoimmune Diseases, Rose and Mackay (eds.), pp. 211-244 (Academic Press, 1998) and in “Animal Models for Autoimmune and Inflamatory, Disease”, in Current Protocols in Immunology, Coligan et al. (eds.), Chapter 15 (Wiley, 1997).

Allergic conditions, e.g. asthma and dermatitis, can also be modeled in rodents. Airway hypersensitivity can be induced in mice by ovalbumin (Tomkinson et al., 2001, J. Immunol. 166:5792-5800) or Schistosoma mansoni egg antigen (Tesciuba et al., 2001, J. Immunol. 167:1996-2003). The Nc/Nga strain of mice show marked increase in serum IgE and spontaneously develop atopic dermatitis-like leisons (Vestergaard et al., 2000, Mol. Med. Today 6:209-210; Watanabe et al., 1997, Int. Immunol. 9:461-466; Saskawa et al., 2001, Int. Arch. Allergy Immunol. 126:239-247).

Injection of immuno-competent donor lymphocytes into a lethally irradiated histo-incompatible host is a classical approach to induce acute GVHD in mice. Alternatively, the parent-B6D2F1 murine model provides a system to induce both acute and chronic GVHD. In this model the B6D2F1 mice are F1 progeny from a cross between the parental strains of C57BL/6 and DBA/2 mice. Transfer of DBA/2 lymphoid cells into non-irradiated B6D2F1 mice causes chronic GVHD, whereas transfer of C57BL/6, C57BL/10 or B10.D2 lymphoid cells causes acute GVHD (Slayback et al., 2000, Bone Marrow Transpl. 26: 931-938; Kataoka et al., 2001, Immunology 103:310-318).

Additionally, both human hematopoietic stem cells and mature peripheral blood lymphoid cells can be engrafted into SCID mice, and these human lympho-hematopoietic cells remain functional in the SCID mice (McCune et al., 1988, Science 241:1632-1639; Kamel-Reid and Dick, 1988, Science 24′:1706-1709; Mosier et al., 1988, Nature 335:256-259). This has provided a small animal model system for the direct testing of potential therapeutic agents on human lymphoid cells. For example, a human-mouse chimera model has been applied to examine the therapeutic potentials of anti-IL-4, anti-IL-13, anti-IL-5, and the double-mutein IL-4 (Tournoy et al., 2001, J. Immunol. 166:6982-6991).

5.8 Assays for Signaling Activity

In certain preferred embodiments, a protein of the invention is capable of inducing one or more hallmarks of signaling through CD30 upon binding to a CD30-expressing lymphocyte. CD30-expressing lymphocytes that can be assayed for a signaling effect of a CD30 binding protein may be cultured cell lines (e.g., Jurkat and CESS, both of which are available from the ATCC; or Karpas 299 and L540, both of which are available from Deutsche Sammlung von Mikroorganismen und Zellkulturen GmbH), or lymphocytes prepared from a fresh blood sample.

In a preferred embodiment, the proteins of the invention are cross-linked prior to assessing their activity on activated lymphocytes. In an exemplary embodiment, where the protein of the invention is an anti-CD30 antibody, the anti-CD30 antibody can be cross-linked in solution. Briefly, one or more dilutions of the anti-CD30 antibody can be titrated into 96-well flat bottom tissue culture plates in the absence or presence of secondary antibodies. Lymphocytes are then added to the plates at approximately 5,000 cells/well. The signaling activity of the antibody can then be assessed as described herein.

Many methods of determining whether a protein induces one or more hallmarks of signaling through CD30 are known to those of skill in the art. Illustrative examples of such methods are described below.

5.8.1 Calcium Release

In one embodiment, a protein of the invention can induce the release of intracellular free Ca²⁺ in Jurkat cells when it is cross-linked, for example with a secondary antibody. The release of intracellular free Ca²⁺ can be measured as described by Ellis et al. (1993, J. Immunol., 151, 2380-2389) or by Mond and Brunswick (1998, Current Protocols in Immunology, Unit 3.9, Wiley).

5.8.2 TRAF Localization

Four TNF receptor-associated factors (TRAFs) including TRAF1, TRAF2, TRAF3, and TRAF5 have been demonstrated to interact with the cytoplasmic tail of CD30 (Gedrich et al., 1996, J. Biol. Chem., 271, 12852-12858; Lee et al., 1996. Proc. Natl. Acad. Sci. USA. 93, 9699-9703; Ansieau et al., 1996, Proc. Natl. Acad. Sci. USA., 93, 14053-14058; Aizawa et al., 1997, J. Biol. Chem. 272, 2042-2045; Tsitsikov et al., 1997, Proc. Natl. Acad. Sci. USA. 94, 1390-1395; Lee er al., 1997, J. Exp. Med., 185, 1275-12S5; Duckett and Thompson, 1997. Genes Dev. 11, 2810-2821). Using co-transfection studies, yeast two-hybrid screening, and GST fusion proteins, the TRAF interacting sites have been mapped to the carboxyl terminal of the cytoplasmic tail of CD30, and the association between CD30 and the TRAFs in the cytosolic phase has been hypothesized to be a key event in the CD30-mediated signal cascade. The interaction between CD30 and TRAF does not appear to require CD30 ligation (Ansieau et al., 1996, Proc. Natl. Acad. Sci. USA., 93, 14053-14058; Aizawa et al., 1997, J. Biol. Chem., 272, 2042-2045). However, cross-linking of CD30 leads to a disappearance of TRAF1 and TRAF2 from the detergent-soluble fractions of cell lysates (Duckett and Thompson, 1997, Genes Dev., 11, 2810-2821; Arch et al., 2000, Biochem. Biophys. Res. Commun., 272, 936-945). The disappearance of TRAF2 is accompanied by a corresponding increase in the quantity of TRAF2 detectable in the detergent-insoluble fraction containing the nuclei (Arch et al., 2000, Biochem. Biophys. Res. Commun., 272, 936-945). Further subcellular localization studies have confirmed that cross-linking of CD30 induces a translocation of TRAF2 from the cytosol to the perinuclear region of cells (Arch et al., 2000, Biochem. Biophys. Res. Commun., 272, 936-945). Such CD30-mediated translocation of TRAF2 is hypothesized to modulate cell survival by regulating the sensitivity of cells to undergo apoptosis induced by other TRAF-binding members of the TNF receptor superfamily (Duckett and Thompson, 1997, Genes Dev., 11, 2810-2821; Arch et al., 2000, Biochem. Biophys. Res. Commun., 272, 936-945).

To determine whether an antibody of the invention induces nuclear translocation of TRAF2, the antibody of the invention is contacted with CD30+ cells and a cross-linking agent, such as a secondary antibody. Confocal microscopy can then be used to compare localization of TRAF2 in cells incubated with the antibody of the invention (plus cross-linking reagent) versus cells not incubated with the antibody of the invention.

In an alternative embodiment, whether an antibody of the invention induces TRAF2 nuclear localization can be assayed by measuring the amount of TRAF2 in various cell fractions, for example on a Western Blot. For example, 2 μg/ml of an antibody of the invention can be incubated with CD30⁺ cells at 0.5×10⁶/ml. The antibody is cross-linked by 20 μg/ml of a secondary antibody (e.g., where the antibody of the invention is a mouse monoclonal antibody, a goat anti-mouse IgG Fc specific antibody (Jackson ImmunoReseach, West Grove, Pa.) can be used as a secondary antibody) at 37° C. and 5% CO₂. At designated time-points (e.g., 2 to 24 hours), 5×10⁶ cells are removed and spun down. After two washes with ice-cold PBS, cells are lysed at 100×10⁶/ml in a lysis buffer (0.15 M NaCl, 0.05 M Tris-HCl, pH 8.0, 0.005 M EDTA, and 0.5% NP-40 or Triton X-100) supplemented with a protease inhibitor cocktail (Roche Diagnositc GmBH, Mannheim, Germany). Lysis is done at 4° C. for 2 hours with constant mixing. After lysis, the detergent-soluble and detergent-insoluble fractions are separated by centrifugation at 14,000×g for 20 minutes. The detergent-soluble fraction is then transferred to a separate tube and an equal volume of 2×SDS-PAGE reducing sample buffer is added to it. An equal volume of 1×SDS-PAGE reducing sample buffer is also added to the detergent-insoluble fraction, i.e., the pellet after centrifugation. Both fractions are heated to 100° C. for 2 minutes. About 10 μl of the fractions from each time point is then resolved by 12% Tris-glycine SDS-PAGE (Invitrogen, Carlsbad, Calif.). Resolved proteins are Western-transferred onto PVDF membranes (Invitrogen), which is blocked with Tris buffer saline (0.05 M Tris-HCl, pH 8.0, 0.138 M NaCl, 0.0027 M KCl) supplemented with 0.05% Tween 20 and 5% BSA. The blots are immunoblotted with an anti-TRAF2 antibody (Santa Cruz, San Diego, Calif.). The presence of TRAF2 protein in the different fractions is detected by horseradish peroxidase (HRP)-conjugated F(ab′)₂ goat anti-rabbit IgG Fc (Jackson ImmunoResearch) and the peroxidase substrate kit DAB (Vector Laboratories, Burlingame, Calif.). Alternatively, the SuperSignal West Pico Chemiluminescent Substrate kit (Pierce, Rockford, Ill.) can also be used for detection.

5.8.3 NF-κB Activation

Another well-defined signal transduction event that can be induced by certain antibodies of the invention is the activation of NF-κB. Anti-CD30 mAbs including M44, M67, and Ber-H2 can activate NF-κB as detected by standard mobility shift DNA-binding assay (McDonald et al., 1995, Eur. J. Immunol., 25, 2870-2876; Ansieau et al., 1996, Proc. Natl. Acad. Sci. USA. 93, 14053-14058: Horie et al., 1998, Int. Immunol., 10, 203-210). Such effect can be observed in Hodgkin cells, T cells, and transfectant expressing CD30 (McDonald et al., 1995, Eur. J. Immunol., 25, 2870-2876: Biswas et al., 1995, Immunity, 2, 587-596; Ansieau er al., 1996, Proc. Natl. Acad. Sci. USA., 93, 14053-14058; Horie et al., 1998, Int. Immunol., 10, 203-210). Initial mapping studies revealed that the interaction between TRAF1, TRAF2, and TRAF5 with the cytoplasmic tail of CD30 was required for the CD30-mediated activation of NF-κB (Lee et al., 1996, Proc. Natl. Acad. Sci. USA., 93, 9699-9703; Ansieau et al., 1996, Proc. Natl. Acad. Sci. USA., 93, 14053-14058; Aizawa et al., 1997, J. Biol. Chem., 272, 2042-2045; Duckett et al., 1997, Mol. Cell. Biol., 17, 1535-1542). More recently, evidence has become available that ligation of CD30 by agonistic mAbs can also activate NF-κB via a TRAF2/5-independent pathway (Horie et al., 1998, Int. Immunol., 10, 203-210). Some of the biological consequences of the CD30-mediated activation of NF-κB include activation of gene transcription (Biswas et al., 1995, Immunity, 2, 587-596; Maggi et al., 1995, Immunity, 3, 251-255) and regulation of cell survival (Mir et al., 2000, Blood, 96, 4307-4312; Horie etal., 2002, Oncogene, 21,2439-2503). Any of these characteristics of NF-κB activation can be assayed to determine whether an antibody of the invention induces one or more hallmarks of CD30 signaling.

Whether NF-κB activation is induced in CD30⁺ cells by an antibody of the invention can be measured by, for example, incubating CD30⁺ cells at 3×10⁶/ml with the antibody at 2 μg/ml, the antibody then cross-linked (e.g., where the antibody is a mouse monoclonal antibody, the antibody can be cross-linked by 20 μg/ml of a goat anti-mouse IgG Fc specific antibody (Jackson ImmunoReseach, West Grove, Pa.)) and the culture incubated at 37° C. and 5% CO₂ for 1 hour with constant shaking. The cell density is adjusted to 1.2×10⁶/ml, and incubation with shaking is carried on for an additional hour. Thereafter, cell density is further reduced to 0.6×10⁶/ml, and cells are incubated for an additional 46 hours at 37° C. and 5% CO₂ without any further shaking. At the end of incubation, nuclear extracts can be prepared from stimulated cells and analyzed for NT-κB activation.

NF-κB activation is assayed by collecting the cells by centrifugation at 1850×g for 20 minutes and then washing them once in 5 packed cell volumes of PBS. The cell pellet is resuspended in 5 packed cell volumes of a hypotonic buffer (0.01 M Hepes, pH 7.9, 0.0015 M MgCl₂, 0.01 M KCl, 0.0002 M phenylmethyl sulphonyl fluoride, 0.0005 M dithiothreitol). Cells are collected by centrifugation at 1850×g for 5 minutes. The pellet is then resuspended in 3 packed cell volumes of the hypotonic buffer and allowed to swell on ice for 10 minutes. After that, swollen cells are homogenized with slow up-and-down strokes in a Dounce homogenizer, using a tight B pestle. Cell lysis is monitored by trypan blue exclusion, and enough strokes should be applied to achieve more than 80% cell lysis. The nuclei are pelleted by centrifugation at 3300×g for 15 minutes. The supernatant (cytoplasmic extract) is removed. The nuclear pellet is then resuspended in ½ packed nuclei volume of a low-salt buffer (0.02 M Hepes, pH 7.9, 25% volume/volume glycerol, 0.0015 M MgCl₂, 0.02 M KCl, 0.0002 M EDTA, 0.0002 M phenylmethyl sulphonyl fluoride, 0.0005 M dithiothreitol). An equal volume of a high-salt buffer (0.02 M Hepes, pH 7.9, 25% volume/volume glycerol, 0.0015 M MgCl₂, 1.2 M KCl, 0.0002 M EDTA, 0.0002 M phenylmethyl sulphonyl fluoride, 0.0005 M dithiothreitol) is then slowly added to the nuclei suspension with gentle stirring to give a final KCl concentration of roughly 0.3 M. The extraction is allowed to continue for 30 minutes with gentle stirring. After extraction, the nuclei are removed by centrifugation at 25,000×g for 30 minutes. The nuclear extraction is then dialyzed against 50 volumes of a dialysis buffer (0.02 M Hepes, pH 7.9, 20% volume/volume glycerol, 0.1 M KCl, 0.0002 M EDTA, 0.0002 M phenylmethyl sulphonyl fluoride, 0.0005 M dithiothreitol) until the conductivity of the nuclear extract is the same as the dialysis buffer. The nuclear extract is centrifuged once more at 25,000×g for 20 minutes to remove residual debris, and the protein concentration of the supernatant is determined by the micro-BCA assay (Pierce).

The presence of NF-κB in nuclear extract of anti-CD30 stimulated cells can be detected by standard mobility shift DNA-binding assay using the Gel Shift Assay System (Promega, Madison, Wis.). A double stranded oligonucleotide probe containing a consensus NF-κB binding motif with the sequence 5′-AGT TGA GGG GAC TTT CCC AGG C-3′ (SEQ ID NO:33) (Lenardo and Baltimore, 1989, Cell, 58, 227-229) is used as the specific probe to detect NF-κB in nuclear extracts. This probe is phosphorylated by T4 polynucleotide kinase and [α-³²P]ATP. The phosphorylated probe is purified by Sepharose G25 spin columns equilibrated with TE buffer (0.01 M Tris-HCl, pH 8.0, 0.001 M EDTA). Purified probed is then precipitated with ammonium acetate and ethanol and then resuspended in 100 μl of TE buffer. Reaction mixtures containing nuclear extracts from anti-CD30-treated cells and control-treated cells are separately combined with the Gel Shift Binding buffer, water and unlabeled competitor probes according to the manufacturers instruction. An unlabeled oligonucleotide containing the NF-κB consensus and an unlabeled irrelevant oligonucleotide are included in the reaction mixture as the sequence-specific and sequence-nonspecific competitors. After incubation for 10 minutes at room temperature, 1 μl of the ³²P-labeled NF-κB consensus oligonucleotide is added to each reaction. The reactions are allowed to continue for an additional 20 minutes at room temperature. At the end of the incubation, 1 μl of a 10× loading buffer (0.25M Tris-HCl, pH 7.5, 40% volume/volume glycerol, 0.2% bromophenol blue) is added to the reactions. The reactions are then loaded into individual wells of a 6% DNA retardation gel (Invitrogen) and resolved at 100 volt for 90 minutes in 0.5×TBE (0.045M Tris-HCl, 0.045 M boric acid, 0.001M EDTA). After electrophoresis, the gel is covered with plastic wrap and exposed to X-ray film at −70° C. to detect the specific interaction between NF-κB and the oligonucleotide containing the NF-κB binding sequence.

5.9 Immune Disorders

The methods of the present invention are useful for treating or preventing an immunological disorder, wherein the immunological disorder is characterized by inappropriate activation of lymphocytes. As used herein, the phrase “immunological disorder” does not encompass immunological cancers such as Hodgkin's Disease and anaplastic large cell lymphoma. Treatment or prevention of an immunological disorder, according to the methods of the present invention, is achieved by administering to a patient in need of such treatment or prevention a protein, preferably an antibody, that binds to activated lymphocytes that are associated with the disease state and exerts a cytotoxic or cytostatic effect on the lymphocytes.

Examples of diseases that can be treated or prevented by the methods of the present invention include, but are not limited to, rheumatoid arthritis, multiple sclerosis, endocrine ophthalmopathy, uveoretinitis, systemic lupus erythematosus, myasthenia gravis, Grave's disease, glomerulonephritis, autoimmune hepatological disorder, autoimmune inflammatory bowel disease, anaphylaxis, allergic reaction, Sjogren's syndrome, juvenile onset (Type I) diabetes mellitus, primary biliary cirrhosis, Wegener's granulomatosis, fibromyalgia, inflammatory bowel disease, polymyositis, dermiatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, presenile dementia, demyelinating diseases, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyly, and telangiectasia), adult onset diabetes mellitus (Type II diabetes), male and female autoimmune infertility, ankylosing spondolytis, ulcerative colitis, Crohn's disease, mixed connective tissue disease, polyarteritis nedosa, systemic necrotizing vasculitis, juvenile onset rheumatoid arthritis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic encephalomyelitis, toxic epidermal necrolysis, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis. Shulman's syndrome, Felty's syndrome, filariasis, cyclitis, chronic cyclitis, heterochronic cyclitis, Fuch's cyclitis, IgA nepliropathy, Henoch-Schonlein purpura, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Eaton-Lambert syndrome, relapsing polychondritis, cryoglobulinemia, Waldenstrom's macroglobulemia, Epstein-Barr virus infection, mumps, Evan's syndrome, and autoimmune gonadal failure.

Accordingly, the methods of the present invention encompass treatment of disorders of B lymphocytes (e.g., systemic lupus erythematosus, Goodpasture's syndrome, rheumatoid arthritis, and type I diabetes), Th₁-lymphocytes (e.g., rheumatoid arthritis, multiple sclerosis, psoriasis, Sjorgren's syndrome, Hashimoto's thyroiditis, Grave's disease, primary biliary cirrhosis, Wegener's granulomatosis, tuberculosis, or acute graft versus host disease), and Th₂-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease).

An alternative way of classifying immunological disease states is by the underlying biological mechanism. The present invention is directed to treatment and prevention of immunological diseases arising by any of the following mechanisms, which are classified into four types:

Anaphylactic reactions. These reactions are mediated by IgE antibodies which bind to receptors on mast cells. When cross-linking occurs with antigens, the IgE antibodies stimulate the mast cells to release a number of pharmacologically active substances that can cause the symptoms characteristic of anaphylaxis. These reactions to antigenic challenge are immediate and potentially life-threatening. Examples of anaphylactic responses include, but are not limited to, allergic rhinitis, gastrointestinal allergy, atopic dermatitis, bronchial asthma and equine heaves and laminitis.

Cytotoxic (cytolytic) reactions. These cell surface reactions result from an interaction of antigen with IgM and/or IgG which activates the complement cascade, leading to the destruction of the cell. Examples of cytolytic reactions include, but are not limited to, leukocytopenia, hemolytic disease of newborn and Goodpasture's disease. Autoimmune disorders that involve cytotoxic/cytolytic reactions are hemolytic anemia, thrombocytopenia and thyroiditis.

Immune complex reactions. Immune complex reactions occur when large complexes of antigen and IgG or IgM accumulate in the circulation or in tissue, fixing complement. Granulocytes are attracted to the site of complement fixation and release damaging lytic enzymes from their granules. An example of this type of reaction is serum sickness. Autoimmune disorders that involve immune complex reactions include systemic lupus erythematosus, chronic glomerulonephritis and rheumatoid arthritis.

Cell-mediated immunity (CMI) reaction, or delayed-type hypersensitivity (DTH). In contrast to the first three types of immune responses, this hypersensitivity response is mediated by T lymphocytes rather than antibodies produced by B lymphocytes. Activated T lymphocytes release cytokines which can result in the accumulation and activation of macrophages, K cells and NK cells, which cause local tissue damage. This reaction can occur 1-2 days after antigenic challenge.

5.10 Gene Therapy

In a specific embodiment, nucleic acids encoding proteins of the invention are administered to treat, inhibit or prevent an immunological disorder. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see, Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; Morgan and Anderson, 1993, Ann. Rev. Biochem. 62:191-217; May, 1993, TIBTECH 1, 1(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the therapeutic comprises nucleic acid sequences encoding an antibody, said nucleic acid sequences being part of expression vectors that express the antibody or fragments or chimeric proteins or have or light chains thereof in a suitable host. In particular, such nucleic acid sequences have promoters operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al., 1989, Nature 342:435-438. In specific embodiments, the expressed antibody molecule is a single chain antibody; alternatively, the nucleic acid sequences include sequences encoding both the heavy and light chains, or fragments thereof, of the antibody.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered its vivo, where it is expressed to produce the encoded product. This can be accomplished by any of numerous methods known in the art, for example by constructing them as part of an appropriate nucleic acid expression vector and administering the vector so that the nucleic acid sequences become intracellular. Gene therapy vectors can be administered by infection using defective or attenuated retrovirals or other viral vectors (see, e.g., U.S. Pat. No. 4,980,286); direct injection of naked DNA; use of microparticle bombardment (e.g., a gene gun; Biolistic, Dupont); coating with lipids or cell-surface receptors or transfecting agents; encapsulation in liposomes, microparticles, or microcapsules; administration in linkage to a peptide which is known to enter the nucleus; administration in linkage to a ligand subject to receptor-mediated endocytosis (see, e.g., Wu and Wu, 1987, J. Biol. Chem. 262:4429-4432) (which can be used to target cell types specifically expressing the receptors); etc. In another embodiment, nucleic acid-ligand complexes can be formed in which the ligand comprises a fusogenic viral peptide to disrupt endosomes, allowing the nucleic acid to avoid lysosomal degradation. In vet another embodiment, the nucleic acid can be targeted in vivo for cell specific uptake and expression, by targeting a specific receptor (see, e.g., PCT Publications WO 92/06 180; WO 92/22635; WO92/20316; WO93/14188, and WO 93/20221). Alternatively, the nucleic acid can be introduced intracellularly and incorporated within host cell DNA for expression by homologous recombination (Koller and Smithies, 1989, Proc. Natl. Acad. Sci. USA 86:8932-8935; Zijlstra et al., 1989, Nature 342:435-438).

In a specific embodiment, viral vectors that contain nucleic acid sequences encoding an antibody of the invention are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy are cloned into one or more vectors, thereby facilitating delivery of the gene into a patient. More detail about retroviral vectors can be found in Boesen et al., 1994, Biotherapy 6:29 1-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Another approach to gene therapy involves transferring a gene, e.g. an AC10 or HeFi-1 gene, to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline. 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to fibroblasts; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, 1992, Cell 71:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

The compounds or pharmaceutical compositions of the invention are preferably tested in vitro, and then in vivo for the desired therapeutic or prophylactic activity, prior to use in humans. For example, in vitro assays to demonstrate the therapeutic or prophylactic utility of an protein or pharmaceutical composition include determining the effect of the protein or pharmaceutical composition on an activated lymphocytes. The cytotoxic and/or cytostatic effect of the protein or composition on the activated lymphocytes can be determined utilizing techniques known to those of skill in the art. Alternatively, in vitro assays which can be used to determine whether administration of a specific protein or pharmaceutical composition is indicated, include in vivo cell culture assays in which activated lymphocytes, including activated lymphocytes from a patient, are grown in culture, and exposed to or otherwise a protein or pharmaceutical composition, and the effect of such compound upon the activated lymphocytes is observed.

5.11 Therapeutic/Prophylactic Administration and Compositions

The invention provides methods of treatment and prophylaxis by administration to a subject of an effective amount of a CD30-binding protein which has a cytotoxic or cytostatic effect on activated lymphocytes (i.e., a protein of the invention), a nucleic acid encoding said CD30-binding protein (i.e., a nucleic acid of the invention), or a pharmaceutical composition comprising a protein or nucleic acid of the invention (hereinafter, a pharmaceutical of the invention).

The outcome of the present therapeutic and prophylactic methods is to at least produce in a patient a healthful benefit, which includes but is not limited to: prolonging the lifespan of a patient, prolonging the onset of symptoms of an immune disorder, and/or alleviating a symptom of the immune disorder after onset of a symptom. Such a healthful benefit can result inhibiting disease progression and/or reducing disease symptoms.

As used herein, the term “prevention” refers to administration of a protein or nucleic acid of the invention to the patient before the onset of symptoms or molecular indications of the immune disorder of interest, for example to an individual with a predisposition or at a high risk of acquiring the immune disorder. In contrast, the term “treatment” refers to administration of a protein or nucleic acid of the present invention to the patient after the onset of symptoms or molecular indications of the immune disorder at any clinical stage.

In a preferred embodiment, the protein of the invention is the monoclonal antibody AC10 or HeFi-1 or a fragment or derivative thereof. In a preferred aspect, a pharmaceutical of the invention comprises a substantially purified protein or nucleic acid of the invention (e.g., substantially free from substances that limit its effect or produce undesired side-effects).

The subject is preferably an animal, and is preferably a mammal, including but not limited to animals such as cows, pigs, horses, chickens, cats, dogs, etc. Most preferably, the subject is human.

Formulations and methods of administration that can be employed are described above; additional appropriate formulations and routes of administration can be selected from among those described herein below.

Various delivery systems are known and can be used to administer a nucleic acid or protein of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction include but are not limited to intradermal, intramuscular, intraperitoneal, intravenous, subcutaneous, intranasal, epidural, and oral routes. Nucleic acids and proteins of the invention may be administered by any convenient route, for example by infusion or bolus injection, by absorption through epithelial or mucocutaneous linings (e.g., oral mucosa, rectal and intestinal mucosa, etc.) and may be administered together with other biologically active agents such as chemotherapeutic agents (see Section 5.2.1). Administration can be systemic or local.

In a specific embodiment, it may be desirable to administer the nucleic acid or protein of the invention by injection, bad means of a catheter, by means of a suppository, or by means of an implant, said implant being of a porous, non-porous, or gelatinous material, including a membrane, such as a sialastic membrane, or a fiber. Preferably, when administering a protein, including an antibody, of the invention, care must be taken to use materials to which the protein does not absorb.

In another embodiment, the compound or composition can be delivered in a vesicle, in particular a liposome (see Langer, 1990, Science 249:1527-1533. Treat et al., 1989, in Liposomes in the Therapy of Infectious Disease and Cancer, Lopez-Berestein and Fidler (eds.), Liss, New York, pp. 353-365; Lopez-Berestein, ibid. pp. 317-327; see generally, ibid.)

In yet another embodiment, the compound or composition can be delivered in a controlled release system. In one embodiment, a pump may be used (see Langer, supra; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, 1974, Langer and Wise (eds.), CRC Pres., Boca Raton, Fla.; Controlled Drug Bioavailability, Drug Product Design and Performance, 1984, Smolen and Ball (eds.), Wiley, New York; Ranger and Peppas, 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

Other controlled release systems are discussed in the review by Langer, 1990, Science 249:1527-1533.

In a specific embodiment where a nucleic acid of the invention is administered, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

As alluded to above, the present invention also provides pharmaceutical compositions (pharmaceuticals of the invention). Such compositions comprise a therapeutically effective amount of a nucleic acid or protein of the invention, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the nucleic acid or protein of the invention, preferably in purified form, together with a suitable amount of carrier so as to provide the form for proper administration to the patient. The formulation should suit the mode of administration.

In a preferred embodiment, the pharmaceutical of the invention is formulated in accordance with routine procedures as a pharmaceutical composition adapted for intravenous administration to human beings. Typically, compositions for intravenous administration are solutions in sterile isotonic aqueous buffer. Where necessary, the pharmaceutical of the invention may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical of the invention is to be administered by infusion, it can be dispensed with an infusion bottle containing sterile pharmaceutical grade water or saline. Where the pharmaceutical of the invention is administered by injection, an ampoule of sterile water for injection or saline can be provided so that the ingredients may be mixed prior to administration.

The amount of the nucleic acid or protein of the invention which will be effective in the treatment or prevention of an immunological disorder can be determined by standard clinical techniques. In addition, in vitro assays may optionally be employed to help identify optimal dosage ranges. The precise dose to be employed in the formulation will also depend on the route of administration, and the stage of immunological disorder, and should be decided according to the judgment of the practitioner and each patient's circumstances. Effective doses may be extrapolated from dose-response curves derived from in vitro or animal model test systems.

5.12 Combination Therapy for Treatment of Immunological Disorders

The nucleic acids and proteins of the invention can be administered together with one or more cytostatic, cytotoxic and/or immunosuppressive agents for the treatment and prevention of immunological disorders. Additionally, combination therapy may include administration of an agent that targets a receptor or receptor complex other than CD30 on the surface of activated lymphocytes. An example of such an agent is a second, non-CD30 antibody that binds to a molecule at the surface of an activated lymphocyte. Another example is a ligand that targets such a receptor or receptor complex. Preferably, such an antibody or ligand binds to a cell surface receptor on activated lymphocytes and enhances the cytotoxic or cytostatic effect of the anti-CD30 antibody by delivering a cytostatic or cytotoxic signal to the activated lymphocytes.

Such combinatorial administration can have an additive or synergistic effect on disease parameters.

With respect to therapeutic regimens, in a specific embodiment., a nucleic acid or protein of the invention is administered concurrently with an immunosuppressive agent or a molecule that targets a lymphocyte cell surface receptor or receptor complex. In another specific embodiment, the immunosuppressive agent or lymphocyte cell surface receptor targeting-agent is administered prior or subsequent to administration of a nucleic acid or protein of the invention, by at least an hour and up to several months, for example at least an hour, five hours, 12 hours, a day, a week, a month, or three months, prior or subsequent to administration of a nucleic acid or protein of the invention.

5.12.1 Immunosuppressive, Cytotoxic and Cytostatic Agents

A useful class of immunosuppressive, cytotoxic or cytostatic agents for practicing the combinatorial therapeutic regimens of the present invention include, but are not limited to, the following non-mutually exclusive classes of agents: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, nitrosoureas, platinols, purine antimetabolites, puromycins, radiation sensitizers, steroids, taxanes, topoisomerase inhibitors, and vinca alkaloids.

Individual immunosuppressive, cytotoxic or cytostatic agents encompassed by the invention include but are not limited to an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramrycin, mitomycin C, mitoxantrone, nitroimidazole, paclitaxel plicamycin, procarbizine, streptozotocin, tenoposide, 6-thioguanine, thioTEPA, topotecan, vinblastine, vincristine, vinorelbine, VP-16 and VM-26.

In a preferred embodiment, the immunosuppressive, cytotoxic or cytostatic agent is an antimetabolite. The antimetabolite can be a purine antagonist (e.g. azothioprine) or mycophenolate mofetil), a dihydrofolate reductase inhibitor (e.g., methotrexate), acyclovir, gangcyclovir, zidovudine, vidarabine, ribavarin, azidothymidine, cytidine arabinoside, amantadine, dideoxyuridine, iododeoxyuridine, poscarnet, and trifluridine.

In another preferred embodiment, the immunosuppressive, cytotoxic or cytostatic agent is tacrolimus, cyclosporine or rapamycin.

In another preferred embodiment, the immunosuppressive agent is a glucocorticoid or glucocorticoid analogue. Examples of glucocorticoids useful in the present methods include cortisol and aldosterone. Examples of glucocorticoid analogues useful in the present methods include prednisone and dexamethasone.

In yet another preferred embodiment, the immunosuppressive agent is an anti-inflammatory agent, such as consisting arylcarboxylic derivatives, pyrazole-containing derivatives, oxicam derivatives and nicotinic acid derivatives. Classes of anti-inflammatory agents useful in the methods of the present invention include cyclooxygenase inhibitors, 5-lipoxygenase inhibitors, and leukotriene receptor antagonists.

Suitable cyclooxygenase inhibitors include meclofenamic acid, mefenamic acid, carprofen, diclofenac, diflunisal, fenbufen, fenoprofen, ibuprofen, indomethacin, ketoprofen, nabumetone, naproxen, sulindac, tenoxicam, tolmetin, and acetylsalicylic acid.

Suitable lipoxygenase inhibitors include redox inhibitors (e.g., catechol butane derivatives, nordihydroguaiaretic acid (NDGA), masoprocol, phenidone, lanopalen, indazolinones, naphazatrom, benzofuranol, alkylhydroxylamine), and non-redox inhibitors (e.g., hydroxythiazoles, methoxyalkylthiazoles, benzopyrans and derivatives thereof, methoxytetrahydropyran, boswellic acids and acetylated derivatives of boswellic acids, and quinolinemethoxyphenylacetic acids substituted with cycloalkyl radicals), and precursors of redox inhibitors.

Other suitable lipoxygenase inhibitors include antioxidants (e.g. phenols, propyl gallate, flavonoids and/or naturally occurring substrates containing flavonoids, hydroxylated derivatives of the flavones, flavonol, dihydroquercetin, luteolin, galangin, orobol, derivatives of chalcone, 4,2°,4′-trihydroxychalcone, ortho-aminophenols, N-hydroxyureas, benzofuranols, ebselen and species that increase the activity, of the reducing selenoenzymes), iron chelating agents (e.g., hydroxamic acids and derivatives thereof. N-hydroxyureas, 2-benzyl-1-naphthol, catechols, hydroxylamines, carnosol trolox C, catechol, naphthol, sulfasalazine, zyleuton, 5-hydroxyanthranilic acid and 4-(omega-arylalkyl)phenylalkanoic acids), imidazole-containing compounds (e.g., ketoconazole and itraconazole), phenothiazines, and benzopyran derivatives.

Yet other suitable lipoxygenase inhibitors include inhibitors of eicosanioids (e.g., octadecatetraenoic, eicosatetraenoic, docosapentaenoic, eicosahexaenoic and docosahexaenoic acids and esters thereof, PGE1 (prostagiandin E1), PGA2 (prostaglandin A2), viprostol, 15-monohydroxyeicosatetraenoic, 15-monohydroxy-eicosatrienoic and 15-monohydroxyeicosapentaenoic acids, and leukotrienes B5, C5 and D5), compounds interfering with calcium flows, phenothiazines, diphenylbutylantines, verapamil, fuscoside, curcumin, chlorogenic acid, caffeic acid, 5,8,11,14-eicosatetrayenoic acid (ETYA), hydroxyphenylretinamide, Ionapalen, esculin, diethylcarbamazine, phenantroline, baicalein, proxicromil, thioethers, diallyl sulfide and di-(1-propenyl) sulfide.

Leukotriene receptor antagonists include calcitriol, ontazolast, Bayer Bay-x-1005, Ciba-Geigy CGS-25019C, ebselen, Leo Denmark ETH-615, Lilly LY-293111, Ono ONO-4057, Terumo TMK-6S8, Boehiringer Ingleheim BI-RM-270, Lilly LY 213024, Lilly LY 264086, Lilly LY 292728, Ono ONO LB457, Pfizer 105696, Perdue Frederick PF 10042, Rhone-Poulenc Rorer RP 66153, SmithKline Beecham SB-201146, SmithKline Beecham SB-201993, SmithKline Beecham SB-209247, Searle SC-53228, Sumitamo SM 15178, American Home Products WAY 121006, Bayer Bay-o-8276, Warner-Lambert CI-987, Warner-Lambert CI-987BPC-15LY 223982, Lilly LY 233569, Lilly LY-255283, MacroNex MNX-160, Merck and Co. MK-591, Merck and CO. MK-886, Ono ONO-LB-448, Purdue Frederick PF-5901, Rhone-Poulenc Rorer RG 14893, Rhone-Poulenc Rorer RP 66364, Rhone-Poulenc Rorer RP 69698, Shionoogi S-2474, Searle SC-41930, Searle SC-50505, Searle SC-51146, Searle SC-52798, SmithKline Beecham SK&F-104493, Leo Denmark SR-2566, Tanabe T-757 and Teijin TEI-1338.

In certain preferred embodiments of the present invention, the immunosuppressive, cytotoxic or cytostatic agent is conjugated to an antibody of the invention rather than being administered separately. Antibody-drug conjugates useful in the present methods are described in Section 5.6, supra.

5.12.2 Lymphocyte Receptor Targeting Agents

Agents that are particularly useful in the present combinatorial methods are molecules that bind to lymphocyte cell surface, preferably against a receptor or receptor complex distinct from CD30. Besides CD30, a wide variety of receptors or receptor complexes expressed on lymphocyte surface are involved in regulating the proliferation, differentiation, and functions of different lymphocyte subsets. Such molecules can be targeted, for example, to provide additional cytostatic or cytotoxic signals to activated lymphocytes.

In one embodiment, suitable receptors for targeting alongside CD30 are immunoglobulin gene superfamily members, including but not limited to CD2, CD3, CD4, CD8, CD19, CD22, CD28, CD79, CD90, CD1152/CTLA-4, PD-1, and ICOS (Barclay et al., 1997, The Leucocyte Antigen FactsBook, 2nd ed, Academic Press; Coyle and Gurtierrez-Ramos, 2001, Nature Immunol. 2:203-209). In another embodiment, TNF receptor superfamily members can be targeted, including but not limited to CD27, CD40, CD95/Fas, CD134/OX40, CD137/4-1BB, TNF-R1, TNFR-2, RANK, TACI, BCMA, osteoprotegerin. Apo2/TRAIL-R1, TRAIL-R2, TRAIL-R3, TRAIL-R4, and APO-3. (Locksley et al., 2001, Cell, 104, 487-501). In yet another embodiment, an integrin can be targeted, including but not limited to CD11a, CD11b, CD11c, CD18, CD29, CD41, CD49a, CD49b, CD49c, CD49d, CD49e, CD49f, CD103, and CD104 (Barclay et al., 1997, The Leucocyte Antigen FactsBook, 2nd ed, Academic Press). In yet other embodiments, a suitable receptor for targeting in addition to CD30 is a cytokine receptor (Fitzgerald et al., 2001, The Cytokine Factsbook. 2nd ed, Academic Press), a chemokine receptor (Luther and Cyster, 2001, Nature Immunol. 2:102-107; Gerard and Rollins. 2001, Nature Immunol. 2: 108-115), a major histocompatibility protein, a lectin (C-type, S-type, or I-type), or a complement control protein.

In one embodiment, agents that bind to these non-CD30 receptors or receptor complexes enhance the cytotoxic or cytostatic effect of the CD30-binding agent (e.g., an anti-CD30 antibody) by delivering a cytostatic or cytotoxic signal to the activated lymphocytes. In combination with the anti-CD30 molecules of the invention, an additive or synergistic effect on growth inhibition or apoptosis can be achieved in the targeted lymphocyte.

In another embodiment, agents against these receptors or receptor complexes need not be growth inhibitory or apoptotic on their own, but, in combination with a CD30-binding agent, an enhanced effect on growth inhibition or apoptosis beyond that induced by the CD30-binding agent alone can be achieved. In certain specific embodiments, the enhanced effect is approximately a 5%, 10% 15%, 20%, 25%, 30%, 40%, 50%, 75%, 100% or greater enhancement in the cytostatic or cytotoxic activity of a given amount or concentration of a CD30-binding agent. In one embodiment, the enhanced effect refers to an approximately 5%, 10% 15%, 20%, 25%, 30%, 40%, 50%, 75% reduction in the ED₅₀ of the CD30-binding agent, i.e., the amount of the CD30-binding agent capable of achieving the same cytotoxic or cytostatic effect is less than what would be required to achieve the same cytotoxic or cytostatic effect in the absence of administration of such agents that bind to receptor or receptor complexes other than CD30.

In one embodiment, targeting a non-CD30 receptor or receptor complex according to the methods of the present invention can be achieved by administering a ligand.

In another embodiment, targeting can be achieved by administering an antibody against the receptor or receptor complex. The antibody can be a polyclonal antibody, a monoclonal antibody, an epitope-binding antibody fragment, or another type of antibody derivative equivalent to those anti-CD30 derivatives described in Sections 5.1 and 5.4, supra. In certain specific embodiments, the antibody is a multivalent antibody or a heteroconjugate comprising a CD30-binding portion, as described in Sections 5.1 and 5.4.

A number of antibodies suitable for co-administration with anti-CD30 are known in the art, as will be recognized by the skilled artisan. Listed below are exemplary, non-limiting examples of such antibodies: the anti-CD2 antibodies include BTI-322 (Medimmune) and UMCD2; the anti-CD3 antibodies OKT3, “SMART” Anti-CD3 (Nuvion™; Protein Design Laboratories) FN18, UCHT1, 145-2C11, and HIT3a; the anti-CD5 antibodies HI211 (6T-003). HISM2 (6T-004), MEM-128 (6T-014), 7.8 (6T-080, OKT1, UCHT2, and BL1a; the anti-CTLA-4 antibodies 11D4. 10AS, 7F8, 4F10, ANC152.2/8H5, and BNI3.1; and the anti-PD-1 antibody J43.

Natural ligands have also been defined for many of the receptors or receptor complexes (Barclay et al., 1997, The Leucocyte Antigen FactsBook, 2nd ed, Academic Press; Coyle and Gurtierrez-Ramos, 2001, Nature Immunol. 2:203-20; Locksley et al., 2001, Cell 104:487-501). Listed below are exemplary, non-limiting examples of such ligands: LFA-3, a ligand for CD2; CD80 and CD86, ligands for CD28 and CTLA-4; PD-L1 and PD-L2, ligands for PD-1; B7RP-1, a ligand for ICOS; CD70, a ligand for CD27; CD154, a ligand for CD40; FasL, a ligand for CD95/Fas; TNFa, a ligand for TNF-R1 and TNF-R2; TRANCE, a ligand for RANK, APRIL, a ligand for TACI; BLYS, a ligand for BCMA, TRAIL, a ligand for TRAIL-R1, -R2, —R3, and R4; and TWEAK, a ligand for APO-3.

5.13 Effective Dose

Toxicity and therapeutic efficacy of the proteins and compositions of the invention can be determined by standard pharmaceutical procedures in cell cultures or experimental animals, e.g., for determining the LD₅₀ (the dose lethal to 0.50% of the population) and the ED₅₀ (the dose therapeutically effective in 50% of the population). The dose ratio between toxic and therapeutic effects is the therapeutic index and it can be expressed as the ratio LD₅₀/ED₅₀. Proteins that exhibit large therapeutic indices are preferred. While proteins that exhibit toxic side effects may be used, care should be taken to design a delivery system that targets such proteins to the site of affected tissue in order to minimize potential damage to uninfected cells and, thereby, reduce side effects.

The data obtained from the cell culture assays and animal studies can be used in formulating a range of dosage for use in humans. The dosage of such proteins lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Generally, the dosage of a protein of the invention in a pharmaceutical of the invention administered to a immunological disorders patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign proteins. Thus, lower dosages of humanized, chimeric or human antibodies and less frequent administration is often possible.

5.14 Formulations

Pharmaceutical compositions for use in accordance with the present invention may be formulated in conventional manner using one or more physiologically acceptable carriers or excipients. Pharmaceutical compositions comprising the anti-CD30 antibodies of the invention may further comprise a second antibody, such as an antibody described in Section 5.12.2, supra, or an immunosuppressive agent, such as one of those enumerated in Section 5.12.1, supra.

Thus, the proteins and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate) lubricants (e.g., magnesium stearate, talc or silica); disintegrants (e.g., potato starch or sodium starch glycolate): or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicles before use. Such liquid preparations may be prepared by conventional means with pharmaceutically acceptable additives such as suspending agents (e.g. sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the proteins for use according to the present invention are conveniently delivered in the form of an aerosol spray presentation from pressurized packs or a nebulizer, with the use of a suitable propellant, e.g., dichlorodifluoromethane, triclilorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The proteins may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The proteins may also be formulated in rectal compositions such as suppositories or retention enemas, e.g., containing conventional suppository bases such as cocoa butter or other glycerides.

In addition to the formulations described previously, the proteins may also be formulated as a depot preparation. Such long acting formulations may be administered by implantation (for example subcutaneously or intramuscularly) or by intramuscular injection. Thus, for example, the proteins may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration preferably for administration to a human.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

The invention is further described in the following examples which are in no way intended to limit the scope of the invention.

### 6. EXAMPLE 1

### Expression of CD30 on the Jurkat Human T Lymphocyte Cell Line

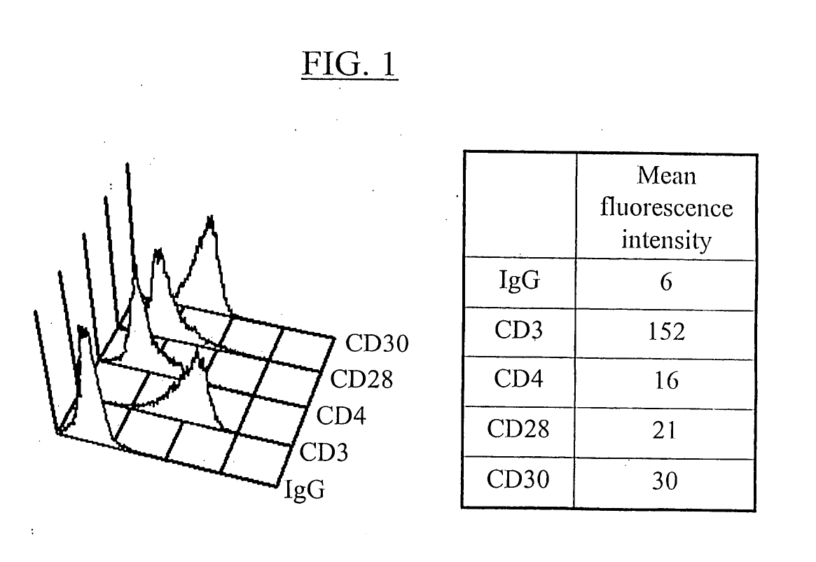

The Jurkat cell line is an acute T leukemia cell line that expresses the CD3/T cell receptor (TCR) complex and other important accessory molecules involved in T cell functions. Sub-lines derived from the original Jurkat line have been applied extensively as model systems to elucidate signaling pathways mediated by a multitude of receptor systems, e.g., CD3/T cell receptor (TCR). A number of signaling pathways in Jurkat T cells initiated upon the ligation of surface receptors have been demonstrated to take place in normal T lymphocytes subjected to antigenic challenge. Jurkat T cells were found to consistently express detectable levels of CD30 (FIG. 1), and therefore they may be a model system to examine the function of CD30 in activated lymphocytes.

### 7. EXAMPLE 2

### Cross-Linking of CD30 on Jurkat T Cells by Anti-CD30 mAbs Inhibited DNA Synthesis

The effect of signaling through CD30 by anti-CD30 on the proliferation of Jurkat T cells was assessed by tritiated thymidine (³H-TdR) incorporation assays. Jurkat cells were treated with soluble anti-CD30 in graded doses or anti-CD30 cross-linked by a secondary cross-linking antibody (Ab). For secondary cross-linking of cAC10, the monoclonal antibody (mAb) was mixed with F(ab′)₂ fragments of goat anti-human (GAM) IgG Fc (Jackson ImmunoResearch, West Groove, Pa.) in culture medium (RPMI-1640, 10% FBS, 2 mM L-glutamine, 1 mM sodium pyruvate, and 0.1 mM non-essential amino acids). For secondary cross-linking of murine anti-CD30 mAb (AC10 and HeFi-1), the mAbs were mixed with F(ab′)₂ fragments of goat anti-mouse (GAM) IgG Fc (Jackson ImmunoResearch) in culture medium. Final ratios of 1:2.5, 1:5, and 1:10 between the primary mAbs and secondary cross-linking antibodies were used. Antibody cocktails were allowed to incubate at room temperature for 15 minutes. Serial dilutions of these antibody cocktails in culture medium were then prepared to achieve the desired final concentrations. One hundred ill of antibody cocktails were then mixed with 100 μl Jurkat cell suspension containing 5000 cells in 96-well tissue culture (TC) plates. Cells were incubated with the antibody cocktails for 44 hours before a 4-hour pulse with 1 μCi of ³H-TdR. Cells were then harvested onto filters, and incorporated tritiated thymidine was measured by scintillation counting. Thymidine incorporation values obtained from the treated cells were then compared to the untreated control cultures.

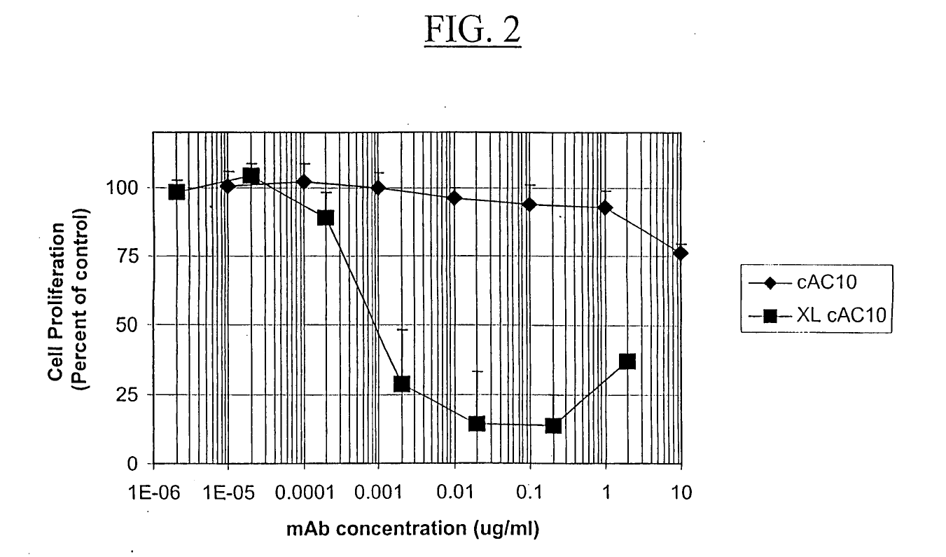

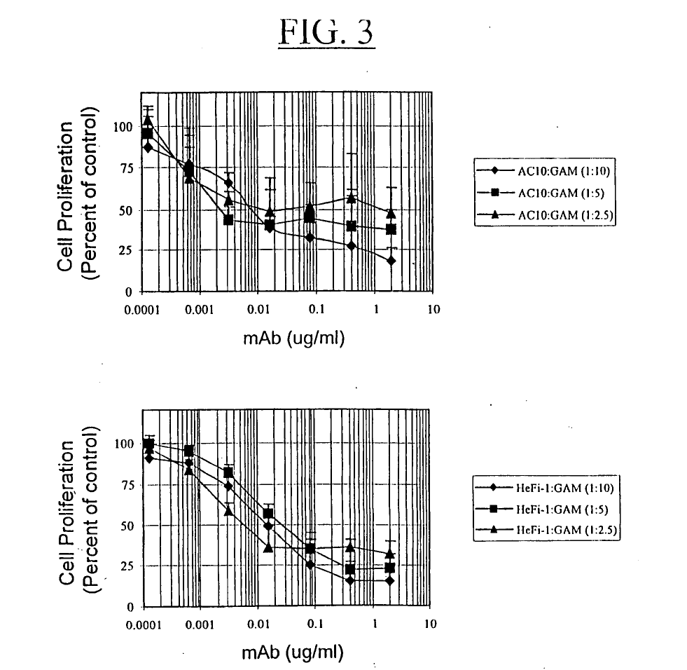

Chimeric AC10 (cAC10) without secondary cross-linking did not inhibit Jurkat cell proliferation at concentrations below 1 μg/ml. Cross-linking cAC10 wraith a secondary Ab lowered the concentration of cAC10 needed to significantly inhibit Jurkat cell proliferation. Maximal inhibition was achieved at concentrations of cAC10 as little as 0.1 μg/ml (FIG. 2). Both AC10 and HeFi-1 inhibited thymidine incorporation in Jurkat cell dose-dependently when a secondary cross-linking antibodies was used (FIG. 3). The use of cross-linking antibodies in vitro likely simulates cross-linking via binding of the Fc portion of antibody to Fc receptors expressed on monocyte, macrophages, B lymphocytes, and NK cells in vivo. A ratio of 1:10 between the primary and secondary cross-linking antibody appeared to be most efficient for proliferation inhibition. Detectable inhibition was observed at primary antibody concentrations of more than 0.001 μg/ml for both AC10 and HeFi-1. Maximal level of inhibition was observed at concentrations greater than 0.1 μg/ml. AC10 and HeFi-1 immobilized onto TC wells were also active in inhibiting Jurkat T cell proliferation (data not shown).

### 8. EXAMPLE 3

### Cross-Linking of CD30 by Anti-CD30 mAbs Induced DNA Fragmentation (Apoptosis) in Jurkat T Cells

The relationship between cell cycle status and DNA replication in Jurkat cells treated with cross-linked anti-CD30 in Abs was further investigated. Four μg/ml of the anti-CD30 mAb AC10 or HeFi-1 were mixed with F(ab′)₂ fragments of GAM IgG Fc (Jackson ImmunoResearch) at final ratios (weight:weight) of 1:4 in culture medium (RPMI-1640, 10% FBS, 2 mM L-glutamine, 1 mM sodium pyruvate, and 0.1 mM non-essential amino acids). The antibody cocktails were allowed to incubate at room temperature for 15 minutes. Serial 1:10 dilutions of these antibody cocktails in culture medium were then prepared. One ml of antibody cocktails was then mixed with 1 ml of Jurkat cell suspension containing 0.25×10⁶ cells in 12-well TC plates.

After 24 or 48 hours of incubation, cells were harvested by centrifugation and resuspended in 2 ml of fresh medium equilibrated at 37° C. Bromodeoxyuridine (BrdU) (Sigma, St. Louis, Mo.) was used to label cells that were actively synthesizing DNA. Twenty μl of 1 mM BrdU dissolved in water were added to the cell suspension, and the incubation continued at 37° C. for 15 minutes. Cells were then spun down, washed once with PBS, and resuspended in 0.5 ml of ice-cold PBS. One ml of 100% ethanol at −20° C. was added drop-wise to the cell suspension for cell fixation. After fixation at 4 C for 30 minutes, fixed cells were spun down. The cell pellets were resuspended in 1 ml of 2 M HCl with 0.5% Triton X-100 (Sigma) and incubated for 30 minutes at room temperature before cells were spun down. One ml of 0.1 M Na₂B₄O₇ at pH 8.5 was used to resuspend the cell pellets and to neutralize the acid for 10 minutes at room temperature. Cells were then pelleted by centrifugation and washed once with PBS containing 0.5% Tween 20 (Sigma) and 1% BSA. Fifty μl of PBS, 0.5% Tween 20, 1% BSA were used to resuspend each cell pellet, to which 25 μl of FITC labeled anti-BrdU (BD Immunocytometry Systems, San Jose, Calif.) were added, and the cell suspension were incubated at room temperature for 30 minutes. Cells were then washed twice with PBS, 0.5% Tween 20, 1% BSA, and then resuspended in 0.5 ml of PBS containing 5 μg/ml of propidium iodide (PI) (Sigma) to quantify DNA contents in cells. DNA synthesis and cell cycle status were then examined by flow cytometry.

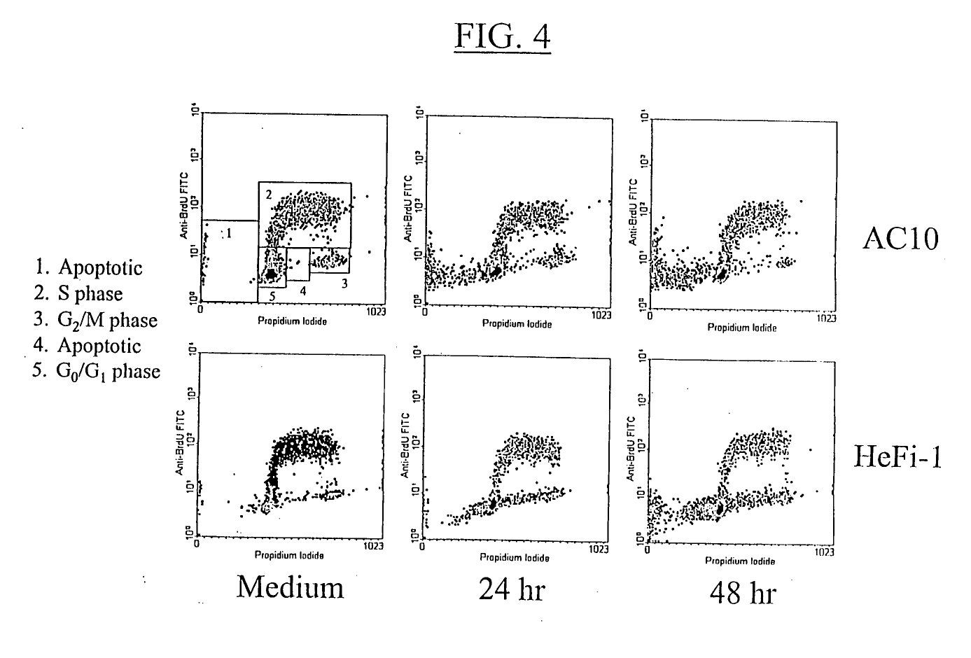

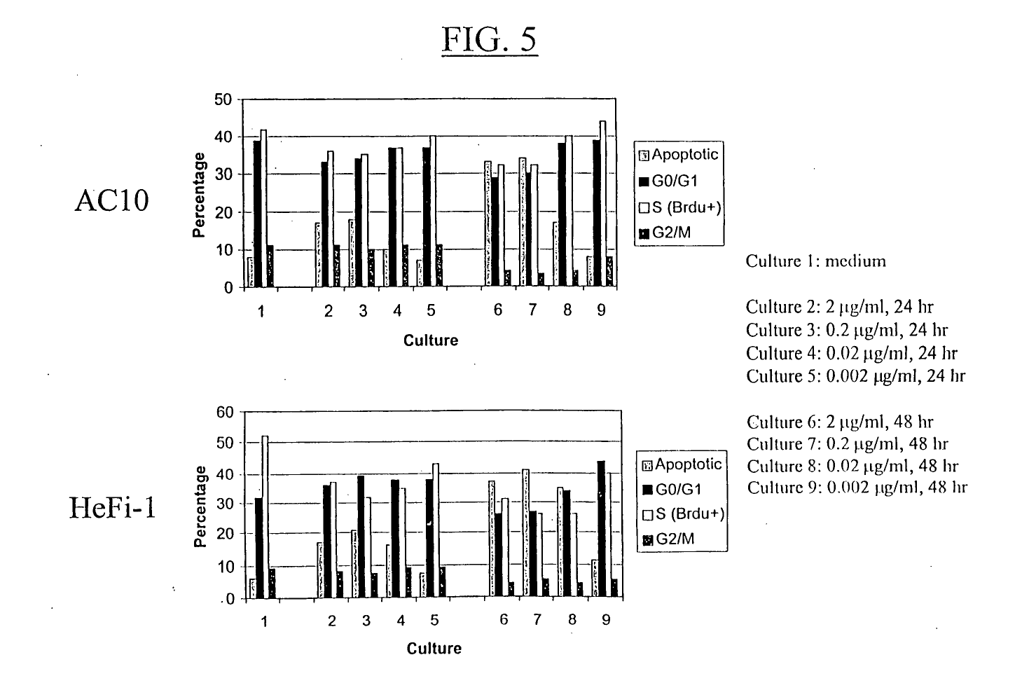

FIG. 4 shows the appearance of Jurkat cells with DNA fragmentation in cultures treated with either AC10 or HeFi-1 cross-linked by a goat anti-mouse IgG Fc antibody. Cells with DNA fragmentation are represented by events showing less than G₀/G₁ phase DNA content (Window 1 in FIG. 4) and events showing less than G₂/M phase DNA content that did not incorporate BrdU, i.e., cells not actively synthesizing DNA, (Window 4 in FIG. 4). DNA fragmentation was detectable after 24 and 48 hours of incubation. Such DNA fragmentation in Jurkat cell is characteristics of apoptosis. These data suggest that the inhibition of proliferation of Jurkat cells shown in FIG. 5 was accompanied by cells undergoing apoptosis in response to CD30 signaling. When the percentages of cells in different parts of the cell cycle from cultures treated with graded doses of AC10 and HeFi-1 were compiled, it was evident that AC10 or HeFi-1 at concentrations of >0.002 μg/ml were able to induce substantial apoptosis in Jurkat cells, especially after 48 hours of treatment (FIG. 5). The appearance of apoptotic cells was paralleled by a corresponding decrease in the percentages of cells in the G₀/G₁, S, and G₂/M phases of the cell cycle (FIG. 5).

### 9. EXAMPLE 4

### Co-Cross-Linking of CD3 with CD30 Enhanced Apoptosis Induced in Jurkat T Cells

Activation-induced cell death (ACID) is a key mechanism used by the immune system to eliminate auto-reactive lymphocytes and effector lymphocytes thereby conferring tolerance to self-antigens and terminating immune responses, respectively. Ligation of CD3/TCR complexes on T lymphocytes or the sIg/B cell antigen receptor complexes on B lymphocytes activates T and B lymphocytes. Depending on the context of antigen receptor ligation and signaling through accessory receptors, lymphocytes can proliferate and differentiate into effector cells or they can undergo apoptosis. A number of accessory receptors, e.g., CD19, CD27, CD28, CD40, CD134/OX40, CD137/4-1BB, and ICOS co-stimulate with the antigen receptors on B or T lymphocytes to promote cellular proliferation and/or differentiation. Hence, they are important for promoting immune responses. On the other hand, signaling through accessory receptors including CD5, CD22, CD152/CTLA4, and PD-1 inhibits lymphocyte proliferation and various lymphocyte functions. Therefore, these receptors may play important roles in the elimination of auto-reactive lymphocytes. They are also involved in the termination of immune responses by suppressing the activities of effector lymphocytes. Defects in regulating signal transduction pathways mediated by accessory receptors may contribute to autoimmune, allergic, and inflammatory diseases.

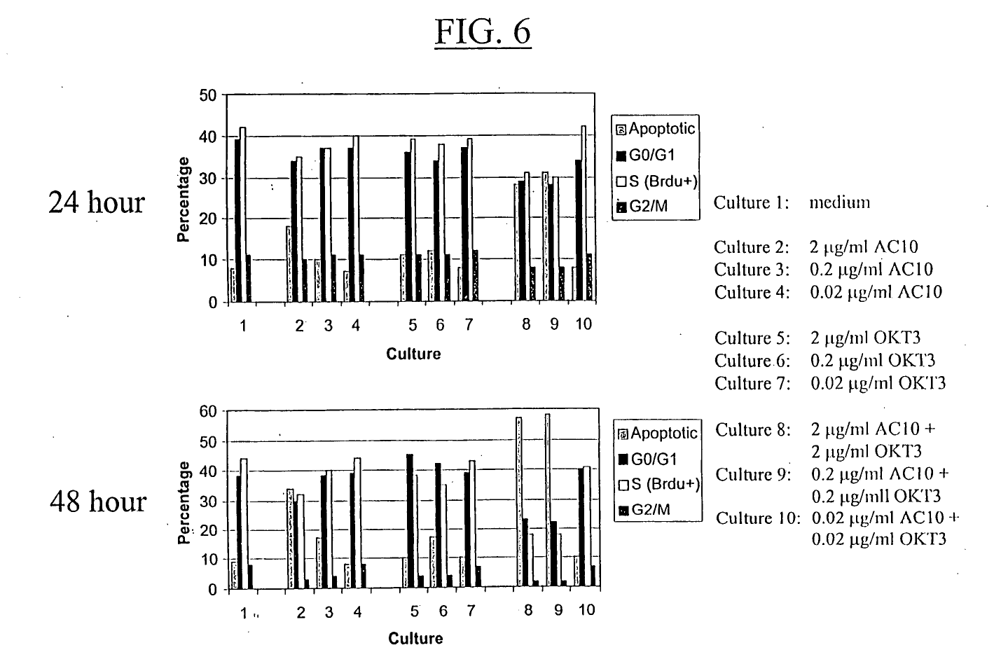

We asked the question if the CD30 signaling pathway cooperates with that of the CD3/TCR complex to regulate apoptosis in lymphocytes. Jurkat T cells were treated with either anti-CD30 mAb alone, anti-CD3 mAb, alone, or a combination of anti-CD30 and anti-CD3 mAbs. Antibodies were cross-linked by goat anti-mouse IgG Fc as described for FIG. 4 and FIG. 5. Cell cycle distribution was determined by BrdU and PI staining as in FIG. 4. Anti-CD30 (AC10) alone induced apoptosis in Jurkat T cells, whereas anti-CD3 (OKT3, ATCC, Manassas, Va.) did not have any effect compared to medium control. However, anti-CD3 greatly enhanced apoptosis induced by anti-CD30 after 24 and 48 hours of incubation. The best synergistic effect between AC10 and OKT3 was observed when both mAbs were used at 0.2 μg/ml (FIG. 6).

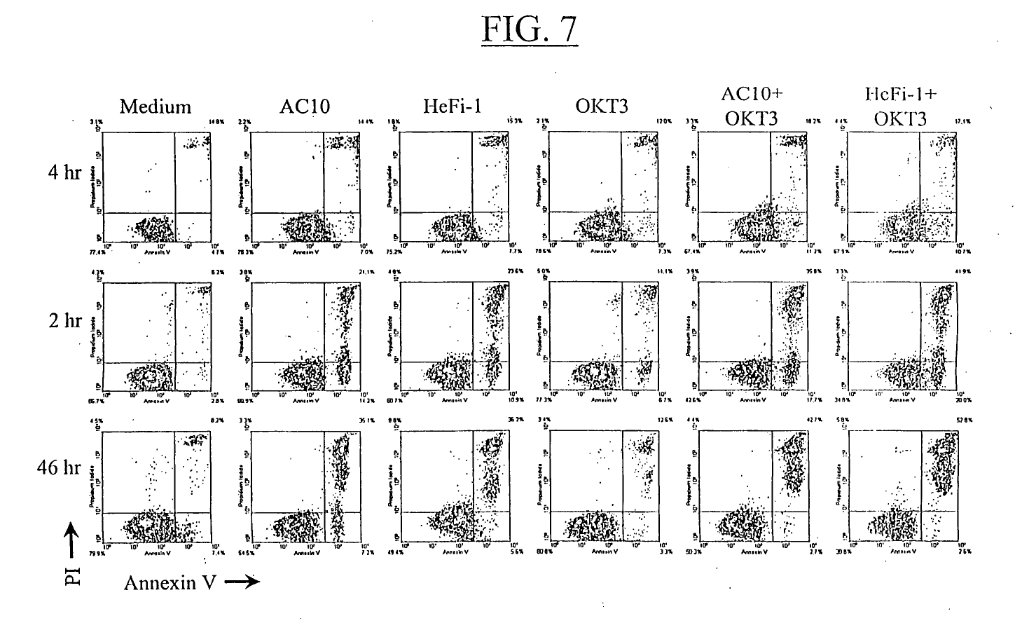

Binding of Annexin V onto the extracellular face of the plasma membrane coupled with membrane permeability to PI was used as a complementary approach to study anti-CD30-induced apoptosis in Jurkat cells. Antibody cocktails containing AC10, HeFi-1. OKT3, AC10+OKT3, or HeFi-1+OKT3 cross-linked with a GAM antibody were prepared as described in Examples 2 and 3. Primary mAbs were used at a final concentration of 2 μg/ml. A 10-fold excess of GA-M antibody was used to cross-link the primary antibodies. After an incubation of 15 minutes at room temperature, antibody cocktails were added to Jurkat cells. At the times designated in FIG. 7, percentages of apoptotic and dead cells in the cultures were determined by Annexin V binding and permeability to PI using the Annexin V-FITC Apoptosis Detection Kit I (BD PharMingen, San Diego, Calif.) according to the manufacturer's instruction. Cells that were Annexin V⁻/PI⁻ were live cells (lower left quadrants in FIG. 7), cells that were Annexin V⁺/PI⁻ were apoptotic cells (lower right quadrants in FIG. 7), and cells that were Annexin V⁺/P⁺ were dead cells (upper right quadrants in FIG. 7). Consistent to the data obtained from PI/BrdU staining shown in FIG. 6 AC10 and HeFi-1 induced detectable apoptosis in Jurkat cells as early as 4 hours after incubation. The presence of an anti-CD3 mAb significantly enhanced the effects of AC10 and HeFi-1. The percentages of apoptotic and dead cells continued to increase during the 48 hours of incubation. These data suggest that the specific combination of anti-CD3 and anti-CD30 mAbs may have significant ability to delete CD30 positive T lymphocytes for controlled suppression of the immune responses in autoimmunity, allergic reactions and chronic inflamatory diseases.

### 10. EXAMPLE 5

### cAC10 Antibody Drug Conjugates (ADCs)

10.1 Synthesis of cAC10-vcMMAE

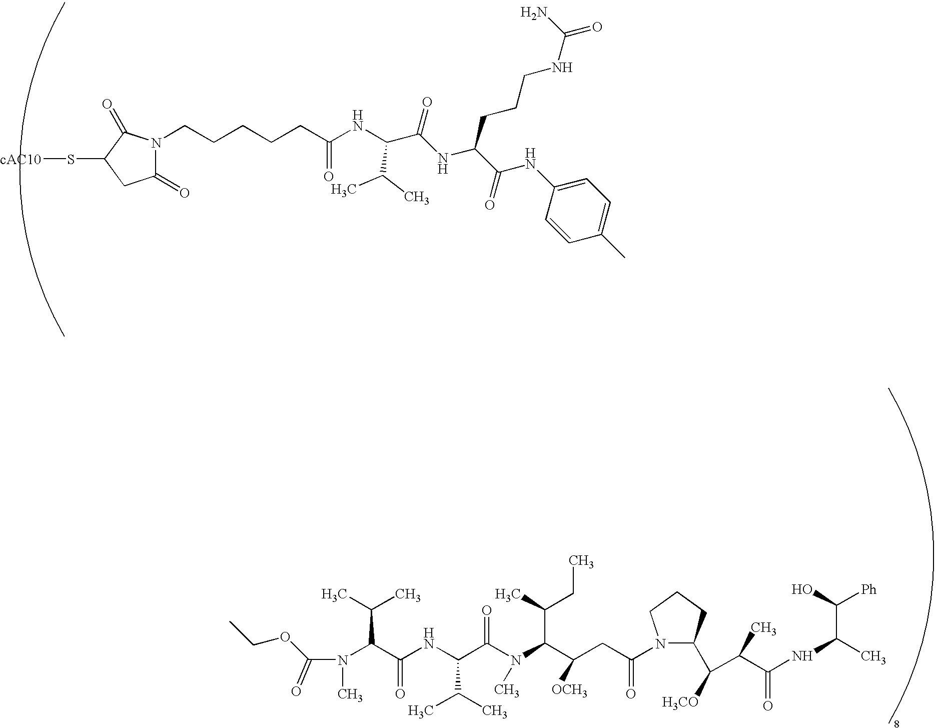

The synthesis of the ADC cAC10-vcMMAE, the general structure of which is depicted above, is described below.

10.1.1 Drug-Linker Compound Synthesis

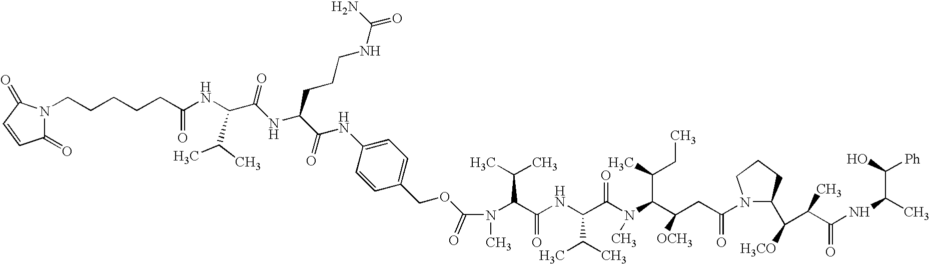

The synthesis of auristatin E has been previously described (Pettit G R, and Barkoczy, J., 1997, U.S. Pat. No. 5,635,483, Pettit, G R, The Dolastatins, Prog. Chem. Org. Nat. Prod., 70, 1-79, 199). The monomethyl derivative of Auristatin E (MMAE) was prepared by replacing a protected form of monomethylvaline for N,N-dimethylvaline in the synthesis of auristatin E (Senter et al., U.S. provisional application No. 60/400,403 filed Jul. 31, 2002, which is incorporated by reference herein in its entirety).

To prepare drug-linker compound, MMAE (1.69 g, 2.35 mmol), maleimidocaproyl-L-valine-L-citrulline-p-aminobenzyl alcohol p-nitrophenylcarbonate (2.6 g, 3.52 mmol, 1.5 eq., prepared as described in Dubowchik. G. M., et al., Bioconjugate Chem. 2002, 13, 855-869) and HOBt (64 mg, 0.45 mmol, 0.2 eq.) were diluted with DMF (25 mL). After 2 min, pyridine (5 mL) was added and the reaction was monitored using reverse-phase HPLC. The reaction was shown to be complete in 24 h. The reaction mixture was concentrated to provide a dark oil, which was diluted with 3 mL of DMF. The DMF solution was purified using flash column chromatography (silica gel, eluant gradient: 100% dichloromethane to 4:1 dichloromethane-MeOH). The relevant fractions were combined and concentrated to provide an oil that solidified under high vacuum to provide a mixture of the desired drug-linker compound and unreacted MMAE as a dirty yellow solid (R_{f} 0.40 in 9:1 dichloromethane-MeOH). The dirty yellow solid was diluted with DMF and purified using reverse-phase preparative-HPLC (Varian Dynamax C₁₈ column 41.4 mm×25 cm, 8μ, 100 Å, using a gradient run of MeCN and 0.1% aqueous TFA at 45 mL/min from 10% to 100% over 40 min followed by 100% MeCN for 20 min) to provide the desired drug-linker compound as an amorphous white powder (Rf 0.40 in 9:1 dichloromethane-MeOH) which was >95% pure by HPLC and which contained less than 1% of MMAE. Yield: 1.78 g (57%); ES-MS m/z 1316.7 [M+H]⁺; UV λₘₐₓ 215, 248 nm.

10.1.2 Conjugate Preparation

Antibody Reduction. To 4.8 mL cAC10 (10 mg/mL) was added 600 μL of 500 mM sodium borate/500 mM NaCl, pH 8.0, followed by 600 μL of 100 mM DTT in water. After incubation at 37° C. for 30 min, the buffer was exchanged by elution over G25 resin equilibrated and eluted with PBS containing 1 mM DTPA (Aldrich). The thiol/Ab value was checked by determining the reduced antibody concentration from the solution's 280 nm absorbance, and the thiol concentration by reaction with DTNB (Aldrich) and determination of the absorbance at 412 nm.

Conjugation of the Reduced Antibody. The reduced mAb was chilled on ice. The drug-linker compound was used as a DMSO solution of known concentration, and the quantity of drug-linker added to the reaction mixture was calculated as follows: L stock solution=V×[Ab]×Fold Excess/[Drug-Linker], where V and [Ab] are the volume and molar concentration of the reduced antibody solution, respectively. 2.3 mL cold PBS/DTPA was added to the reduced antibody solution. 133.6 uL of 7.5 mM drug-linker compound stock solution was diluted into 1.47 mL acetonitrile. The acetonitrile drug-linker solution was chilled on ice, then added to the reduced antibody solution. The reaction was terminated after 1 hr by the addition of a 20 fold molar excess of cysteine over maleimide. The reaction mixture was concentrated by centrifugal ultrafiltration and purified by elution through de-salting G25 in PBS. cAC10-vcMMAE was then filtered through 0.2 micron filters under sterile conditions and immediately frozen at −80C. cAC10-vMMAE was analyzed for 1) concentration, by UV absorbance; 2) aggregation, by size exclusion chromatography; 3) drug/Ab, by measuring unreacted thiols with DTNB, and 4) residual free drug, by reverse phase HPLC.

10.2 Synthesis of cAC10-fkAEFP

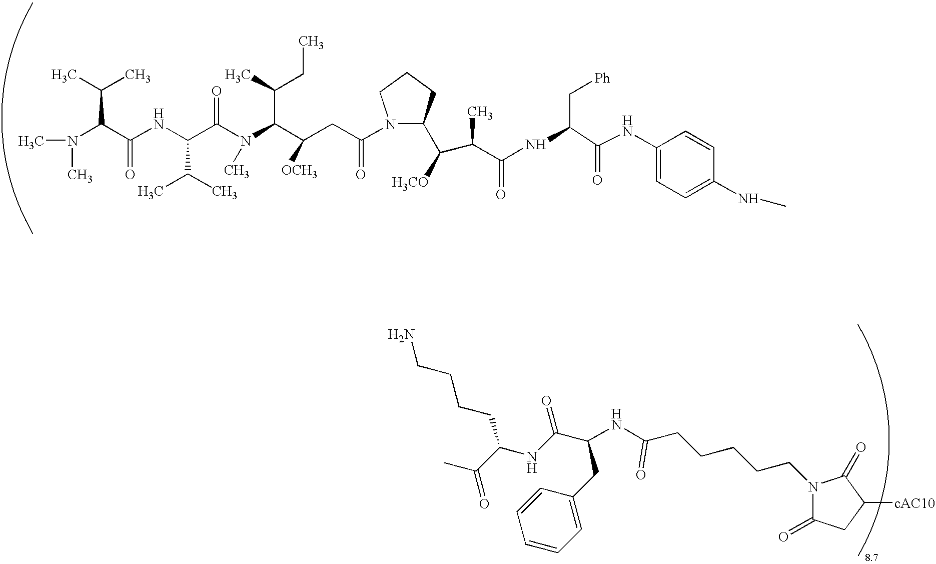

The synthesis of the ADC cAC10-fkAEFP, the general structure of which is depicted above, is described below.

10.2.1 AEFP Synthesis

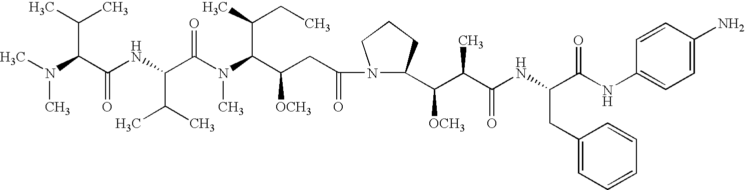

Boc-phenylalanine (1.0 g, 3.8 mmol) was added to a suspension of 1,4-diaminobenzene.HCl (3.5 g, 19.0 mmol, 5.0 eq.) in triethylamine (10.7 mL, 76.0 mmol, 20 eq.) and dichloromethane (50 mL). To the resulting solution was added DEPC (3.2 mL, 19.0 mmol, 5.0 eq.) via syringe. HPLC showed no remaining Boc-phe after 24 h. The reaction mixture was filtered, and the filtrate was concentrated to provide a dark solid. The dark solid residue was partitioned between 1:1 EtOAc-water, and the EtOAc layer was washed sequentially with water and brine. The EtOAc layer was dried and concentrated to provide a dark brown/red residue that was purified using HPLC (Varian Dynamax column 41.4 mm×25 cm, 5μ, 100 Å, using a gradient run of MeCN and water at 45 mL/min form 10% to 100% over 40 min followed by 100% MeCN for 20 min). The relevant fractions were combined and concentrated to provide a red-tan solid intermediate. Yield: 1.4 g (100%); ES-MS mil: 355.9 [M+H]⁺; UV λₘₐₓ 215, 265 nm; ¹H NMR (CDCl₃) δ 7.48 (1H, br s), 7.22-7.37 (5H, m), 7.12 (2H, d, J=8.7 Hz), 7.61 (2H, d, J=8.7 Hz), 5.19 (1H, br s), 4.39-4.48 (1H, m), 3.49 (2H, s), 3.13 (2H, d, J=5.7 Hz), 1.43 (9H, s).

The red-tan solid intermediate (0.5 g, 1.41 mmol) and diisopropylethylamine (0.37 mL, 2.11 mmol, 1.5 eq.) were diluted with dichloromethane (10 mL), and to the resulting solution was added Fmoc-Cl (0.38 g, 1.41 mmol). The reaction was allowed to stir, and a white solid precipitate formed after a few minutes. Reaction was complete according to HPLC after 1 h. The reaction mixture was filtered, and the filtrate was concentrated to provide an oil. The oil was precipitated with EtOAc, resulting in a reddish-white intermediate product which was collected by filtration and dried under vacuum. Yield: 0.75 g (93%); ES-MS m/z 578.1 [M+H]⁺, 595.6 [M+NH₄]⁺.

The reddish-white intermediate (0.49 g, 0.85 mmol), was diluted with 10 mL of dichloromethane, and then treated with 5 mL of trifluoroacetic acid. Reaction was complete in 30 nin according to reverse-phase HPLC. The reaction mixture was concentrated and the resulting residue was precipitated with ether to provide an off-white solid. The off-white solid was filtered and dried to provide an amorphous powder, which was added to a solution of Boc-Dolaproine (prepared as described in Tetrahedron, 1993, 49(9):1913-1924) (0.24 g, 0.85 mmol) in dichloromethane (10 mL). To this solution was added triethylamine (0.36 mL, 2.5 mmol, 3.0 eq.) and PyBrop (0.59 g, 1.3 mmol, 1.5 eq.). The reaction mixture was monitored using reverse-phase HPLC. Upon completion, the reaction mixture was concentrated, and the resulting residue was diluted with EtOAc, and sequentially washed with 10% aqueous citric acid, water, saturated aqueous sodium bicarbonate, water and brine. The EtOAc layer was dried (MgSO₄), filtered, and concentrated. The resulting residue was purified using flash column chromatography (silica gel) to provide an off-white powdered intermediate. Yield: 0.57 g (88%); ES-MS m/z 764.7 [M+NH₄]⁺; UV λₘₐₓ 215, 265 nm; ¹H NMR (DMSO-d₃) δ 10.0-10.15 (1H, m), 9.63 (1H, br s), 8.42 (½H, d, J=8.4 Hz), 8.22 (½H, d J=8.4 Hz), 7.89 (2H, d, J=7.2 Hz), 7.73 (2H, d, J=7.6,Hz), 7.11-7.55 (13H, m), 4.69-4.75 (1H, m), 4.46 (2H, d, J=6.8 Hz), 4.29 (1H, t, J=6.4 Hz), 3.29 (3H, s), 2.77-3.47 (7H, m), 2.48-2.50 (3H, m), 2.25 (⅔H, dd, J=9.6, 7.2 Hz), 1.41-1.96 (4H, m), 1.36 (9H, s), 1.07 (1H, d, J=6.4 Hz, rotational isomer), 1.00 (1H, d, J=6.4 Hz, rotational isomer).

The white solid intermediate (85 mg, 0.11 mmol) and Me-val-val-dil-O-t-butyl (55 mg, 0.11 mmol, prepared as described in Pettit et al. J. Chem. Soc. Perk. 1, 1996, 859) were diluted with dichloromethane (5 mL), and then treated with 2.5 mL of trifluoroacetic acid under a nitrogen atmosphere for two hours at room temperature. The reaction completion was confirmed by RP-HPLC. The solvent was removed in vacuo and the resulting residue was azeotropically dried twice with toluene, then dried under high vacuum for 12 hours.

The residue was diluted with dichloromethane (2 mL), diisopropylethylamine (3 eq.) was added, followed by DEPC (1.2 eq.). After the reaction was completed, the reaction mixture was concentrated under reduced pressure, the resulting residue was diluted with EtOAc, and washed sequentially with 10% aqueous citric acid, water, saturated aqueous sodium bicarbonate, and brine. The EtOAc layer was dried, filtered and concentrated to provide a yellow oil.

The yellow oil was diluted with dichloromethane (10 mL) and to the resulting solution diethylamine (5 mL) was added.” According to HPLC, reaction was completed after 2 h. The reaction mixture was concentrated to provide an oil. The oil was diluted with DMSO, and the DMSO solution was purified using reverse phase preparative-HPLC (Varian Dynamax column 21.4 mm×25 cm, 5μ, 100 Å, using a gradient run of MeCN and 0.1% TFA at 20 mL/min from 10% to 100% over 40 min followed by 100% MeCN for 20 min). The relevant fractions were combined and concentrated to provide the desired drug as an off-white solid. Overall yield: 42 mg (44% overall); ES-MS m/z 837.8 [M+H]⁺, 858.5 [M+Na]⁺; UV λₘₐₓ 215, 248 nm.

10.2.2 Linker Synthesis

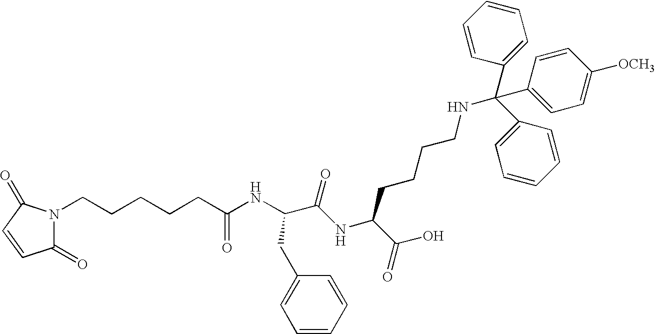

The linker compound maleimidocaproyl-L-phenylalanine-L-lysine(MMT) was prepared as described in Dubowchik et al., 2002, Bioconjugate Chem. 13:855-896.

10.2.3 Preparation of Drug-Linker Compound

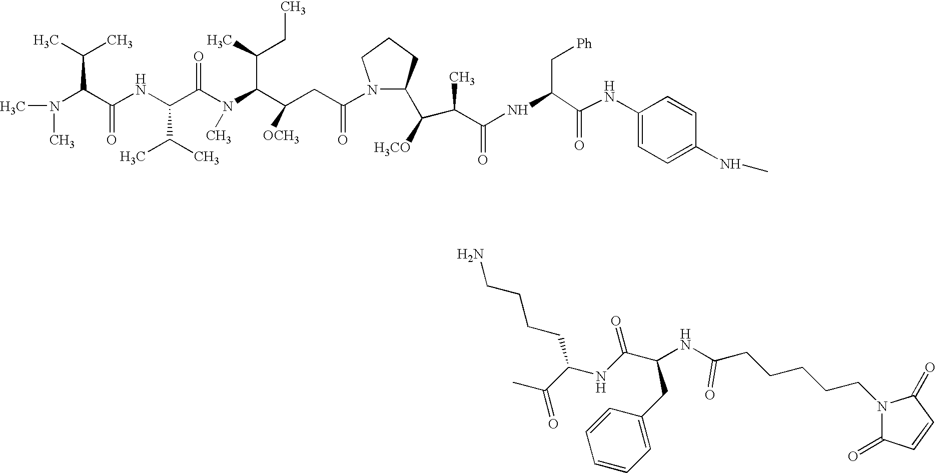

The drug of Section 10.2.1 (9 mg, 10.8 mmol) and the linker from section 10.2.2 (5.2 mg, 10.8 μmol) were diluted with dichloromethane (1 mL) and to the resulting solution was added HATU (6.3 mg, 16.1 mmol, 1.5 eq.), followed by pyridine (1.3 μL, 16.1 μmol, 1.5 eq.). The reaction mixture was allowed to stir under argon atmosphere while being monitored using HPLC. The reaction was complete after 6 h. The reaction mixture was concentrated and the resulting residue was diluted with DMSO. The DMSO solution was purified using reverse phase preparative HPLC (Varian Dynamax column 21.4 m=×25 mm, 5 m, 100 Å, using a gradient run of MeCN and Et3N—CO2 (pH 7) at 20 mL/min from 10% to 100% over 40 min followed by 100% MeCN for 20 min) and the relevant fractions were combined and concentrated to provide an off-white solid intermediate which was >95% pure according to HPLC.

The off-white solid intermediate was diluted with dichloromethane (2 mL) and the resulting solution was treated with TFA (0.5 mL). The reaction was monitored using HPLC, and was complete in 2 h. The reaction mixture was concentrated, and the resulting residue was diluted with DMSO and purified under the same conditions as described in Example 13. The relevant fractions were combined and concentrated to provide the desired drug-linker compound as an off-white powder. Yield: 14.9 mg (90%); ES-MS m/z 1304.6 [M+H]⁺; UV λₘₐₓ 215, 275 nm.

10.2.4 Conjugate Preparation

Antibody Reduction. To 3.0 mL cAC10 (10 mg/mL) was added 375 μL of 500 mM sodium borate/500 mM NaCl, pH 8.0, followed by 375 μL of 100 mM DTT in water. After incubation at 37° C. for 30 min., the buffer was exchanged by elution over G25 resin equilibrated and eluted with PBS containing 1 mM DTPA (Aldrich). The thiol/Ab value was checked by determining the reduced antibody concentration from the solution's 280 nm absorbance, and the thiol concentration by reaction with DTINB (Aldrich) and determination of the absorbance at 412 nm.

Conjugation of the Reduced Antibody. The reduced mAb was chilled on ice. The drug-linker compound was used as a DMSO solution of known concentration, and the quantity of drug-linker added to the reaction mixture was calculated as follows: L stock solution=V×[Ab]×Fold Excess/[Drug-Linker], where V and [Ab] are the volume and molar concentration of the reduced antibody solution, respectively. 2.2 mL cold PBS/DTPA was added to the reduced antibody solution, followed by 1.47 mL DMSO, and the mixture chilled on ice. 140.0 uL of 7.6 mM drug-linker compound stock solution was then added to the reduced antibody/ DMSO solution. The reaction was terminated after 1 hr by the addition of a 20 fold molar excess of cysteine over maleimide. The reaction mixture was concentrated by centrifugal ultrafiltration and purified by elution through de-salting G25 in PBS. cAC10-fkAEFP was then filtered through 0.2 micron filters under sterile conditions and immediately frozen at −80° C. cAC10-fkAEFP was analyzed for 1) concentration, by UV absorbance; 2) aggregation, by size exclusion chromatography; 3) drug/Ab, by measuring unreacted thiols by treatment with DTT, followed by DTNB, and 4) residual free drug, by reverse phase HPLC.

10.3 Synthesis of cAC10-vcAEFP:

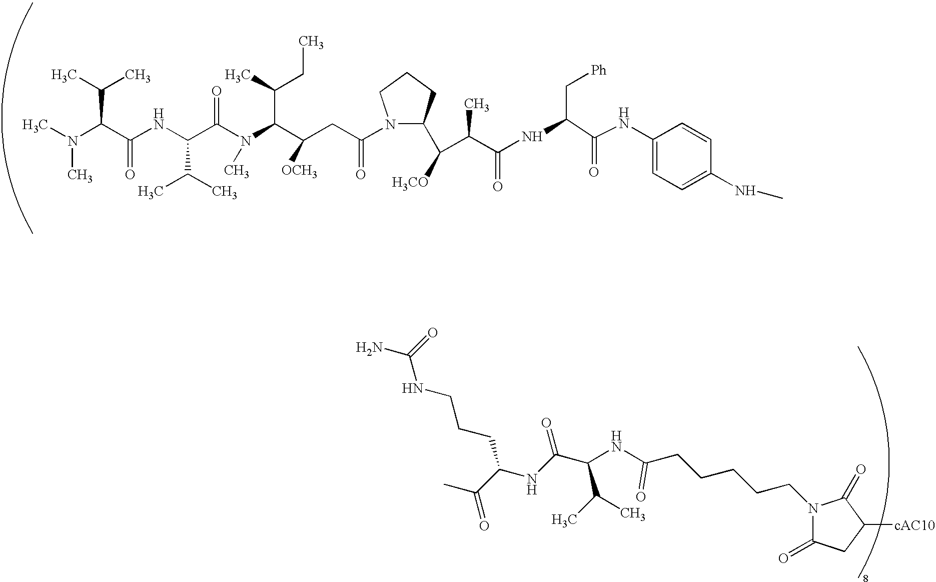

The synthesis of the ADC cAC10-vcAEFP, the general structure of which is depicted above, is described below.

10.3.1 Drug Synthesis

The drug was synthesized as illustrated above herein at Section 10.2.1.

10.3.2 Preparation of Drug-Linker Compound

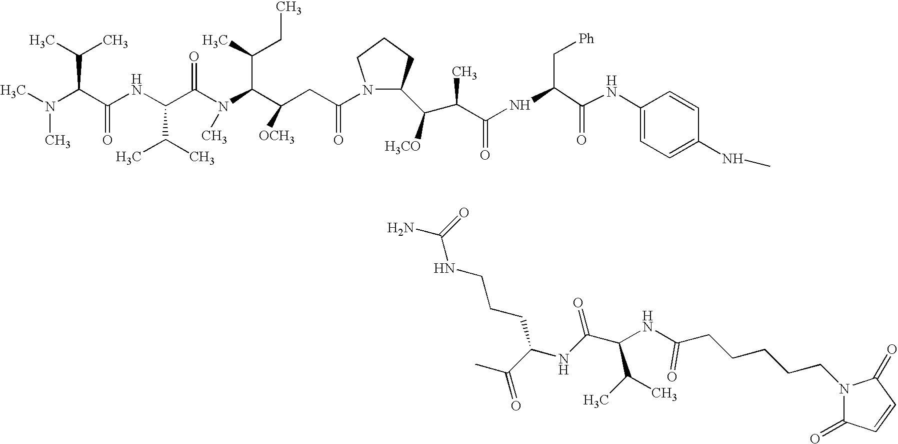

The trifluoroacetate salt of the drug of Section 10.2.1 (0.37 g, 0.39 mmol, 1.0 eq.) and Fmoc-val-cit (0.30 g, 0.58 mmol, 1.5 eq., prepared according to Dubowchik et al., Bioconjugate Chem. 2002, 13, 855-896) were diluted with DMF (5 mL, 0.1 M), and to the resulting solution was added pyridine (95 μL, 1.2 mmol, 3.0 eq.). HATU (0.23 g, 0.58 mmol, 1.5 eq.) was then added as a solid and the reaction mixture was allowed to stir under argon atmosphere while being monitored using HPLC. The reaction progressed slowly, and 4 h later, 1.0 eq. of diisopropylethylamine was added. Reaction was complete in 1 h. The reaction mixture was concentrated in vacuo and the resulting residue was purified using prep-HPLC (Varian Dynamax C18 column 41.4 mm×25 cm, 5μ, 100 Å, using a gradient run of MeCN and 0.1% aqueous TFA at 45 mL/min from 10% to 100% over 40 min followed by 100% MeCN for 20 min) to provide a faint pink solid intermediate.

The pink solid intermediate was diluted with DMF (30 mL) and to the resulting solution was added diethylamine (15 mL). Reaction was complete by HPLC in 2 h. The reaction mixture was concentrated and the resulting residue was washed twice with ether. The solid intermediate was dried under high vacuum and then used directly in the next step.

The solid intermediate was diluted with DMF (20 mL) and to the resulting solution was added 6-(2,5-dioxy-2,5-dihydro-pyrrol-1-yl)-hexanoic acid 2.5-dioxy-pyrrolidin-1-yl ester (0.12 g, 0.39 mmol, 1.0 eq.) (EMCS, Molecular Biosciences Inc., Boulder, Colo.). After 4 d, the reaction mixture was concentrated to provide an oil which was purified using prep-HPLC (Varian Dynamax C18 column 41.4 mm×25 cm, 5μ, 100 Å, using a gradient run of MeCN and 0.1% aqueous TFA at 45 mL/min from 10% to 100% over 40 min followed by 100% MeCN for 20 min) to provide the desired drug-linker compound as a white flaky solid. Yield: 0.21 g (38% overall); ES-MS m/z 1285.9 [M+H]+; 13.07.8 [M+Na]+; UV λₘₐₓ 215, 266 nm.

10.3.3 Conjugate Preparation

Antibody Reduction. To 3.0 mL cAC10 (10 mg/mL) was added 375 μL of 500 mM sodium borate/500 mM NaCl, pH 8.0, followed by 375 μL of 100 mM DTT in water. After incubation at 37° C. for 30 min., the buffer was exchanged by elution over G25 resin equilibrated and eluted with PBS containing 1mM DTPA (Aldrich). The thiol/Ab value was checked by determining the reduced antibody concentration from the solution's 280 nm absorbance, and the thiol concentration by reaction with DTNB(Aldrich) and determination of the absorbance at 412 nm.

Conjugation of the Reduced Antibody. The reduced mAb was chilled on ice. The drug-linker compound was used as a DMSO solution of known concentration, and the quantity of drug-linker added to the reaction mixture was calculated as follows: L stock solution=V×[Ab]×Fold Excess/[Drug-Linker], where V and [Ab] are the volume and molar concentration of the reduced antibody solution, respectively. 2.2 mL cold PBS/DTPA was added to the reduced antibody solution., followed by 1.47 mL DMSO, and the mixture chilled on ice. 140.0 μL of 7.6 mM drug-linker compound stock solution was then added to the reduced antibody/DMSO solution. The reaction was terminated after 1 hr by the addition of a 20 fold molar excess of cysteine over maleimide. The reaction mixture was concentrated by centrifugal ultrafiltration and purified by elution through de-salting G25 in PBS. cAC10-vcAEFP was then filtered through 0.2 micron filters wider sterile conditions and immediately frozen at −80° C. cAC10-vcAEFP was analyzed for 1) concentration, by UV absorbance; 2) aggregation, by size exclusion chromatography; 3) drug/Ab, by measuring unreacted thiols by treatment with DTT, followed by DTNB, and 4) residual free drug, by reverse phase HPLC.

10.4 Synthesis of cAC10-fkMMAE

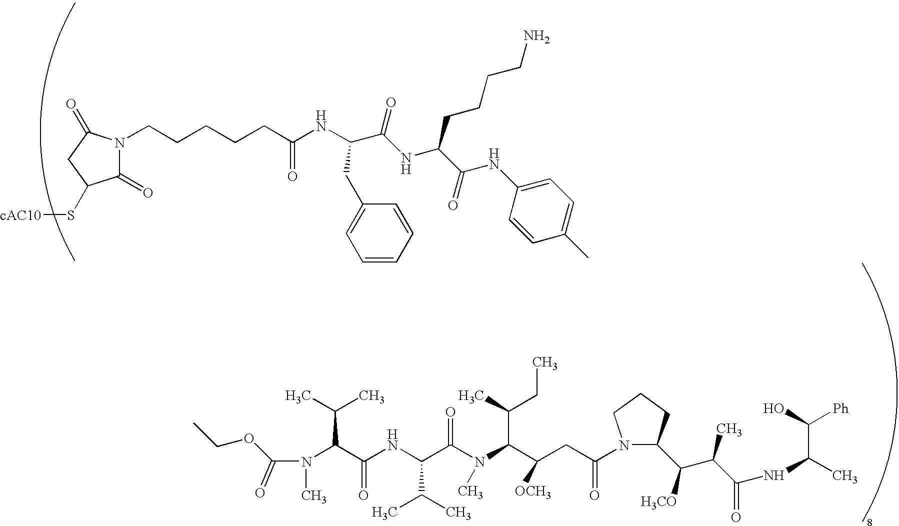

The synthesis of the ADC cAC10-fkMMAE, the general structure of which is depicted above, is described below.

10.4.1 Preparation of Drug-Linker Compound

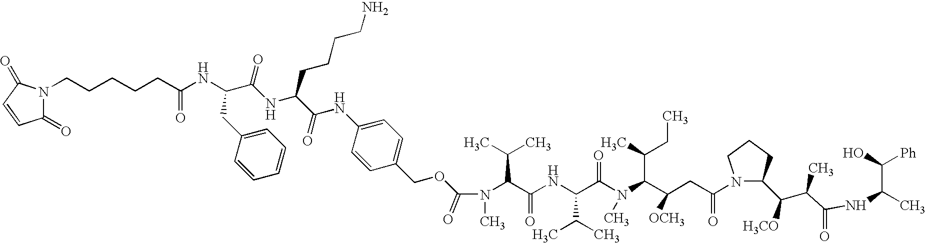

The synthesis of auristatin E has been previously described (Pettit G R, and Barkoczy, J., 1997 U.S. Pat. No. 5,635,483, Pettit, G. R., Prog. Chem. Org. Nat. Prod., 70, 1-79, 199). The monomethyl derivative of Auristatin E (MMAE) was prepared by replacing a protected form of monomethylvaline for N,N-dimethylvaline in the synthesis of auristatin E (Senter et al., U.S. provisional application No. 60/400,403 filed Jul. 31, 2002, which is incorporated by reference herein in its entirety).

MMAE (100 mg, 0.14 mmol), the linker maleimidocaproyl-L-phenylalanine-L-lysine(MMT)-p-aminobenzyl alcohol p-nitrophenylcarbonate (160 mg, 0.15 mmol, 1.1 eq., prepared as described in Dubowchik et al., Bioconjugate Chem. 2002, 13, 855-869), and HOBt (19 mg, 0.14 mmol, 1.0 eq.) were diluted with DMF (2 mL). After 2 min, pyridine (0.5 mL) was added and the reaction mixture was monitored using reverse-phase HPLC. Neither MMAE nor the linker was detected after 24 h. The reaction mixture was concentrated, and the resulting residue was purified using reverse phase preparative-HPLC (Varian Dynamax column 21.4 mm×25 cm, 5μ, 100 Å, using a gradient run of MeCN and Et₃N—CO₂ (pH 7) at 20 mL/min from 10% to 100% over 40 min followed by 100% MeCN for 20 min). The relevant fractions were pooled and concentrated to provide an off-white solid intermediate. ES-MS m/z 1608.7 [M+H]⁺.

The off-white solid intermediate was diluted with MeCN/water/TFA in an 85:5:10 ratio, respectively. The reaction mixture was monitored using HPLC and was complete in 3 h. The reaction mixture was directly concentrated and the resulting residue was purified using reverse phase preparative-HPLC (Varian Dynamax column 21.4 mm×25 cm, 5μ, 100 Å, using a gradient run of MeCN and 0.1% TFA at 20 mL/min from 10% to 100% over 40 min followed by 100% MeCN for 20 min). The relevant fractions were combined and concentrated to provide the desired drug-linker compound as an off-white powder. Yield: 46 mg (32% overall); ES-MS m/z 1334.8 [M+H]⁺; UV λₘₐₓ 215, 256 nm.

10.4.2 Conjugate Preparation

Antibody Reduction. To 4.8 mL cAC10 (10 mg/mL) was added 600 uL of 500 mM sodium borate/500 mM NaCl, pH 8.0, followed by 600 uL of 100 mM DTT in water. After incubation at 37° C. for 30 min, the buffer was exchanged by elution over G25 resin equilibrated and eluted with PBS containing 1 nM DTPA (Aldrich). The thiol/Ab value was checked by determining the reduced antibody concentration from the solution's 280 nm absorbance, and the thiol concentration by reaction with DTNB (Aldrich) and determination of the absorbance at 412 nm.

Conjugation of the Reduced Antibody. The reduced mAb was chilled on ice. The drug-linker compound was used as a DMSO solution of known concentration, and the quantity of drug-linker added to the reaction mixture was calculated as follows: L stock solution=V×[Ab]×Fold Excess/[Drug-Linker], where V and [Ab] are the volume and molar concentration of the reduced antibody solution, respectively. 2.3 mL cold PBS/DTPA was added to the reduced antibody solution. 133.6 uL of 7.5 drug-linker compound stock solution was diluted into 1.47 mL acetonitrile. The acetonitrile drug-linker solution was chilled on ice, then added to the reduced antibody solution. The reaction was terminated after 1 hr by the addition of a 20 fold molar excess of cysteine over maleimide. The reaction mixture was concentrated by centrifugal ultrafiltration and purified by elution through de-salting G25 in PBS. cAC10-fkMMAE was then filtered through 0.2 micron filters under sterile conditions and immediately frozen at −80C. cAC10-fkMMAE was analyzed for 1) concentration, by UV absorbance; 2) aggregation, by size exclusion chromatography; 3) drug/Ab, by measuring unreacted thiols with DTNB, and 4) residual free drug, by reverse phase HPLC.

10.5 Results

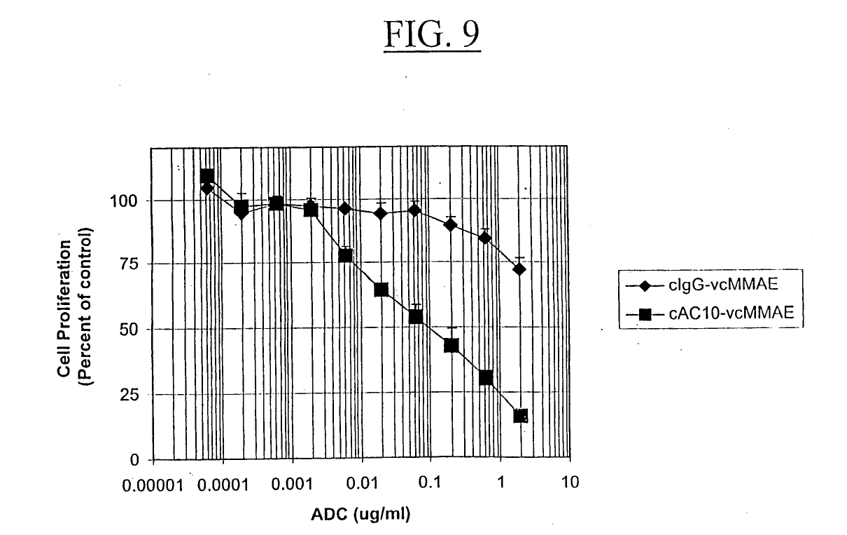

The ability of cAC10-vcMMAE to inhibit Jurkat T cell proliferation was then examined. Five thousand Jurkat T cells in 100 μl of medium were seeded in 96-well TC plates. Graded concentrations of cAC10-vcMMAE or a control non-binding control IgG (cIgG)-vcMMAE in 100 μl of medium were added to Jurkat cells to achieve the final concentrations given in FIG. 9. After 80 hours of incubation, cellular DNA synthesis was assessed by a 16 hour ³H-TdR pulse. Chimeric AC10-vcMMAE at concentrations higher than 0.001 μg/ml significantly inhibited DNA synthesis. The IC₅₀ was approximately 0.1 μg/ml. The control cIgG-vcMMAE did not show significant growth inhibitory action at concentrations lower than 0.1 μg/ml. FIG. 2 shows that soluble, unconjugated cAC10 had growth inhibitory effect on Jurkat cells at concentration higher than 1 μg/ml. Collectively, these data suggest that cAC10 is effective in delivering a cytotoxic drug to CD30⁺ target cells, and that the cAC10-vcMMAE ADC efficiently internalized upon binding to target cells.

### 11. EXAMPLE 6

### Activation of Normal Human Peripheral Blood Mononuclear Cells (PBMC) Induced CD30 Expression on T Lymphocytes

Human PBMC obtained from normal donors through apheresis were the source of human T lymphocytes. Immobilized anti-CD3 and anti-CD28 mAbs were used to activate T lymphocytes and induce them to proliferate. To immobilize mAbs, one μg/ml of each an anti-CD3 mAb (OKT3) and an anti-CD28 mAb (B-T3, DiaClone Research, Besancon, France, or 9.3, Hara et al., 1985, J. Exp. Med., 161, 1513-1524) in PBS were incubated at 37 C for 2 hours in TC wells or flasks. Unbound mAbs were removed by two washes with PBS. T cell activation was initiated by seeding PBMC into mAb-coated TC wells or flasks at a concentration of 0.5×10⁶ cells/ml in culture medium (RPMI-1640, 10% FBS, 2 mM L-glutamine, 1 mM sodium pyruvate, and 0.1 mM non-essential amino acids). In some experiments, recombinant human IL-2 (rhIL-2) (Chiron, Emeryville, Calif.) at a final concentration of 100 IU/ml was also included.

T cell activation and CD30 expression were monitored by multi-color flow cytometric analysis. For detecting CD30 expression, purified anti-CD30 (AC10) was labeled with the fluorescent probe Alexa Fluor (AF) (Molecular Probes, Eugene, Oreg.) according to the manufacturer's instruction. Before antibody staining, activated PBMC were washed once in staining medium (PBS, 1% bovine serum albumin (BSA), 0.02% sodium azide) and pelleted. A 25 μl cocktail of either AF labeled anti-CD3+ phycoerythrin (PE) labeled anti-CD4 (BD PharMingen, San Diego, Calif.)+Cy-Chrome (Cy) labeled anti-CD8 (BD PharMingen) or fluorescein isothiocyanate (FITC) anti-CD25 (BD PharMingen)+PE labeled anti-CD4 (BD PharMingen)+Cy labeled anti-CD8 (BD PharMingen) in staining medium was used to resuspend a 0.2×10⁶ cell pellet. Cells were incubated on ice for 20 minutes, washed 2 times w ith staining medium, and fixed in 1% paraformaldehyde in PBS before being analyzed on a FACscan (BD Bioscience, San Jose, Calif.).

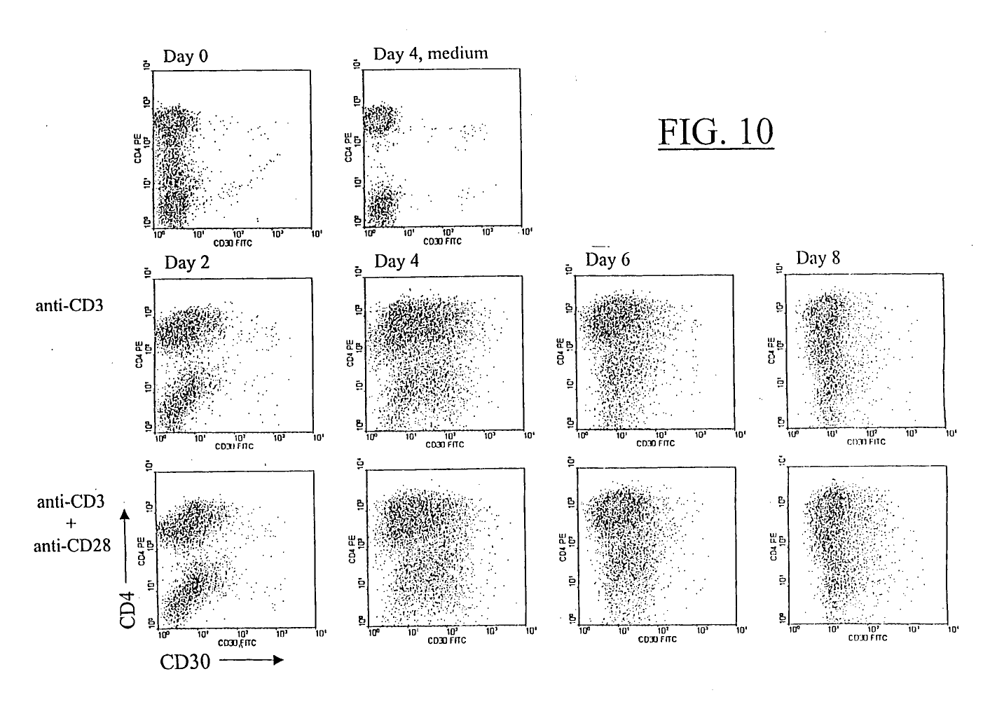

As shown in FIG. 10, very few T lymphocytes in unstimulated PBMC expressed CD30. Activation of PBMC with either anti-CD3+anti-CD28 or anti-CD3+anti-CD28′ IL-2 induced a time-dependent expression of CD30 on T lymphocytes. In contrast, PBMC maintained in medium alone did not show induction of CD30 expression. Increased expression of CD30 was detected on both CD4⁺ and CD4⁻ (containing CD8⁺ cells, data not shown) cells. The peak of expression on both CD4⁺ and CD8⁺ T lymphocytes was around 4 days after stimulation. The level declined thereafter to almost basal after 8 days in culture.

### 12. EXAMPLE 7

### cAC10 ADCS Inhibited Proliferation of Activated Normal T Lymphocytes

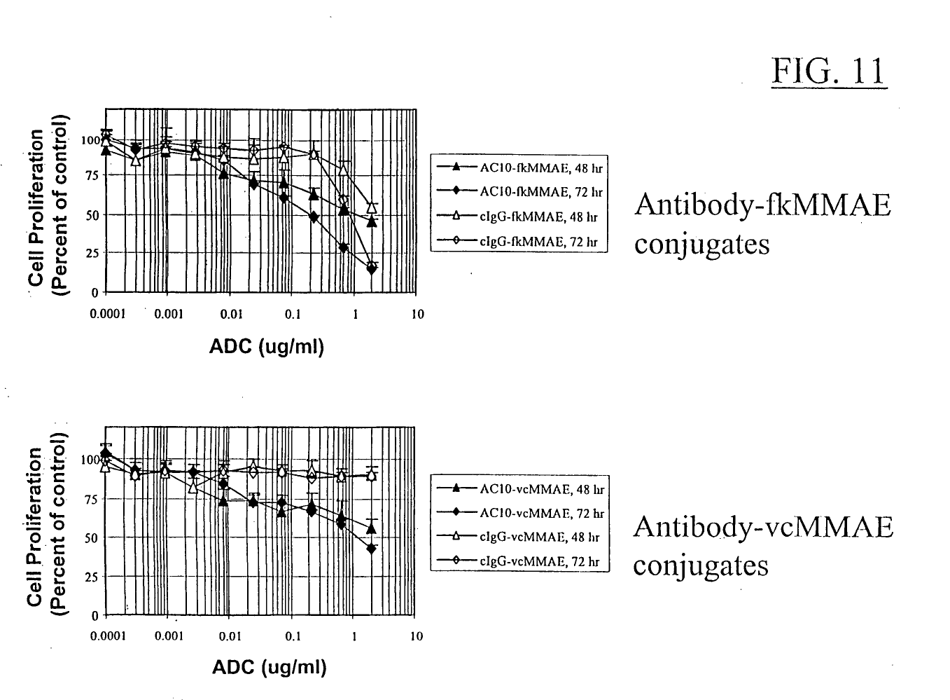

The ability of cAC10 ADCs to inhibit the proliferation of activated normal T lymphocytes was then examined. Normal PBMCs were activated by anti-CD3 and anti-CD28 mAbs as described in Example 6. On day 3 during culture, which was close to the peak of CD30 induction (FIG. 10), activated PBMC were harvested. A portion of the cells was used for flow cytometric analysis to confirm the CD30 expression on both CD4 and CDS cells. The rest of the cells were pelleted and resuspended in fresh medium containing 200 IU/ml of rhIL-2 at 50,000 cells/ml. One hundred μl of this cell suspension (5,000 cells) were transferred to each well of 96-well TC plates. One hundred μl of graded concentrations of cAC10-fkMMAE, cAC10-vcMMAE, or the corresponding non-binding cIgG ADCs in medium supplemented with 200 IU/ml of rhIL-2 were added to culture wells to achieve the concentrations indicated in FIG. 11. Cells were cultured for an additional 48 or 72 hours with a pulse of ³H-TdR during the last 16 hours to assess cellular DNA synthesis. Similar to its activity on Jurkat T cells, cAC10 ADCs inhibited the proliferation of activated PBMC (FIG. 11). Inhibitory effects were detectable when cAC10 ADCs were present at concentrations higher than 0.01 μg/ml. A 72-hour incubation with either ADC resulted in more profound growth inhibition than a 48-hour incubation. Chimeric AC10-fkMMAE appeared to be more active than cAC10-vcMMAE.

### 13. EXAMPLE 8

### Memory T Lymphocytes were More Sensitive to cAC10 ADC than Naïve T Lymphocyte

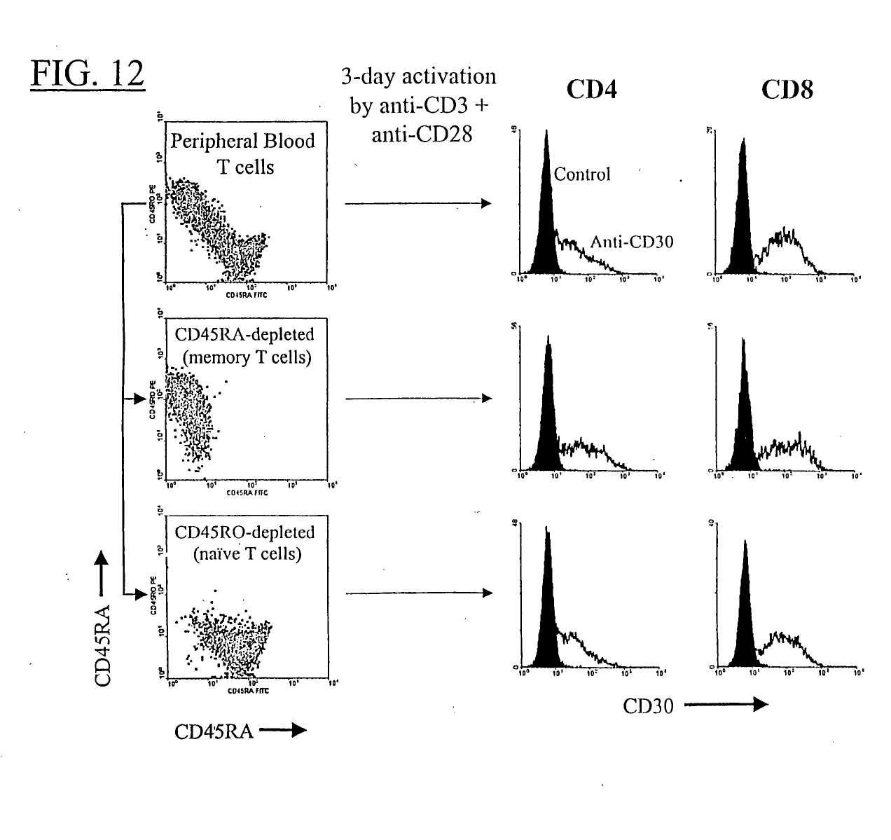

As PBMC are made up of different subsets of lymphocytes, the response of one cellular subset toward cAC10 ADCs may easily be masked by other subsets. In order to examine this possibility, total T lymphocytes, naïve and memory T lymphocytes were enriched from PBMC by negative immuno-selection. Briefly, PBMCs were incubated with one of the following antibody cocktails, containing saturating quantities of antibodies, at a final concentration of 20×10⁶ cells/ml on ice for 20 minutes. To enrich for total T lymphocytes, the antibody cocktail contained anti-CD 14 (BD PharMingen), anti-CD16 (BD PharMingen), and anti-CD20 (BD PharMingen). To enrich for naïve T lymphocytes (CD45RO⁻), the antibody cocktail contained anti-CD14, anti-CD16, anti-CD20, and anti-CD45RO (BD PharMingen). To enrich for memory T lymphocytes (CD45RA⁻), the antibody cocktail contained, anti-CD14, anti-CD16, anti-CD20, and anti-CD45RA (BD PharMingen). All antibody cocktails were prepared in medium supplemented with 10% FBS. After antibody binding, cells were washed twice with ice-cold culture medium and resuspended to a concentration of 20×10⁶ cells/ml in culture medium. Antibody-bound cells were removed from the cell suspension by Dynabeads M450 goat anti-mouse IgG paramagnetic beads (Dynal, Oslo. Norway). Before addition to the cell suspension, Dynabeads M450 goat anti-mouse IgG were washed twice by culture medium and resuspended to a concentration of 60×10⁶/ml in culture medium. Equal volumes of cell and paramagnetic beads were mixed and rotated for 2 hours at 4° C. The cell/paramagnetic bead suspension was diluted 2-fold with culture medium. Unbound paramagnetic beads and paramagnetic bead-cell conjugates were attracted to the side of the culture by the application of a magnet. Unbound cells, enriched for the subsets described above were removed. Flow cytometric analysis was conducted to confirm the enrichment as shown on the left column of FIG. 12.

Activation-induced expression of CD30 on different T cell subsets was then examined. Briefly, total, naïve, and memory T lymphocytes obtained from immuno-selection were stimulated by immobilized anti-CD3 and anti-CD28 antibodies in the presence of 200 IU/ml of IL-2 as described in Example 6. After 72 hours of incubation, the expression of CD30 on both CD4 and CDS cells in the different cultures was determined by flow cytometry, also as outlined in Example 6. Expression of CD30 was detected on both CD4 and CDS cells regardless of whether the starting population was total, naïve, or memory T lymphocytes (FIG. 12). The levels of CD30 on memory CD4⁺ and CD8⁺ cells were slightly higher than the other subsets on a consistent basis.

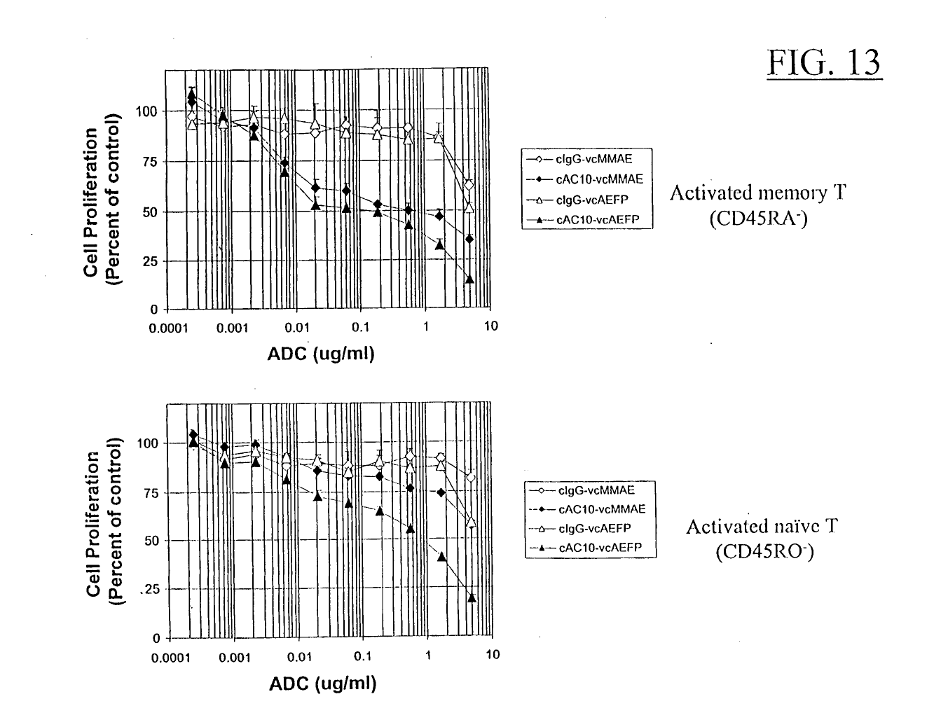

The responses of activated naïve (CD45RO⁻) and memory T lymphocytes (CD45RA⁻) to cAC10 ADCs were then examined. Three-day activated naïve and memory T lymphocytes were plated at 5,000 cells/well in 96-well TC plates in a final volume of 200 μl of medium containing 200 IU/ml of rhIL-2 and cAC10 ADCs or cIgG ADCs at final concentrations indicated in FIG. 13. Cells were incubated for an additional 72 hours with the last 16 hours pulsed with ³H-TdR to assess cellular DNA synthesis. Whereas cIgG ADCs did not have any significant effect on T cell proliferation at concentrations lower than 2 μg/ml, proliferation of activated memory T lymphocytes was significantly inhibited by either cAC10-vcMMAE or cAC10-vcAEFP at concentrations higher than 0.007 μg/ml. By contrast, activated naïve T lymphocytes were relatively refractory to cAC10-vcMMAE. The cAC10-vcAEFP conjugate was found to be more effective than the cAC10-vcMMAE conjugate on both T cell subsets. Notwithstanding, naïve T lymphocytes were still less sensitive to cAC10-vcAEFP than memory T lymphocytes (FIG. 13). The difference in sensitivity between naïve and memory T lymphocytes toward the cAC10 ADCs was probably not a consequence of antigen densities, as both activated naïve and memory T lymphocytes were found to express comparable levels of CD30 (FIG. 12). The growth inhibitory activity of cAC10-vcMMAE reported in FIG. 11 probably reflected the combined responses of naïve and memory T lymphocytes. More importantly, these results suggest that cAC10-vcMMAE may be able to selectively suppress CD30⁺ memory T lymphocyte proliferation while having minimal effects on CD30⁻ naïve T lymphocytes. Effector T lymphocytes implicated in the pathogenesis of autoimmune, inflamatory, and allergic diseases are usually antigen-primed, and they belong to the memory T lymphocyte subset. Accordingly, application of cAC10-vcMMAE in therapeutic intervention may have the advantage of only targeting antigen-primed T lymphocytes. On the other hand, application of cAC10-vcAEFP may be preferred in situations in which suppressing the proliferation of both activated naïve and memory T lymphocytes is desired, e.g., during transplant rejection.

### 14. EXAMPLE 9

### Allogeneic Stimulation of Normal Human T Lymphocytes Induced CD30 Expression

Effector T lymphcytes implicated in the pathogenesis of autoimmune, allergic, and inflamatory responses have usually gone through multiple rounds of antigenic stimulation and expansion. During this process of chronic activation, they continue to carry out effector functions including cytokine secretion and cytolytic responses to induce tissue damages. It is therefore important to examine if T lymphocytes that have undergone repeated rounds of activation and expansion can still express CD30, and how anti-CD30 mAbs and their ADCs can be applied to inhibit the expansion of such chronically activated CD30⁺ T lymphocytes. One of such T cell lines was generated against allogeneic stimulator cells. CD4⁺ lymphocytes were enriched from PBMC by the depletion of CD8⁺ cells as detailed in Example 9. Briefly, PBMC were incubated in culture medium containing a saturating concentration of an anti-CD8 mAb (BD PharMingen) on ice for 20 minutes. Cells were then washed twice with ice-cold culture medium. Anti-CD8 bound cells were removed from the cell suspension by Dynabeads M450 goat anti-mouse IgG paramagnetic beads. Unbound cells, enriched for CD4′ cells, were analyzed by flow cytometry for CD4 expression.

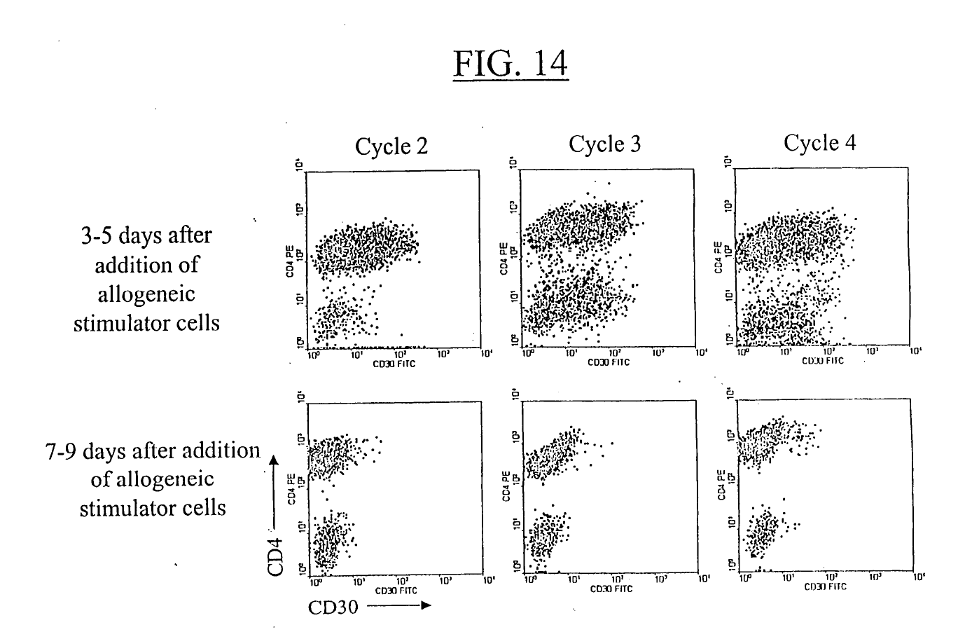

PBMC (5×10⁶), enriched for CD4⁺ cells, were co-cultured with an equal number of gamma-irradiated (2700-5000 rads) allogeneic Daudi cells (ATCC). EL-2 was added to a final concentration of 400 IU/ml on day 4, and the culture was allowed to continue until day 14. Viable cells were then re-stimulated and expanded with irradiated Daudi cells at a T cell to Daudi ratio of 1:3 in the presence of 200 IU/ml of IL-2. This was the beginning of cycle 2 (FIG. 14). After 7 to 9 days in culture, T lymphocytes were re-stimulated again with irradiated Daudi cell to start the following round of expansion. Expression of CD30 was examined 3 to 4 days and 7 to 9 days after the addition of allogeneic stimulator cells. As shown in FIG. 7, CD30 was induced in each round of allogeneic stimulation on both the CD4 and CD4- cells. The levels of CD30 expressed gradually decline toward the end of each stimulation cycle. These results confirm that antigen-primed T lymphocytes retain the capacity to express CD30 when they are challenged with antigenic stimulation.

### 15. EXAMPLE 10

### Generation of T Lymphocyte Clones

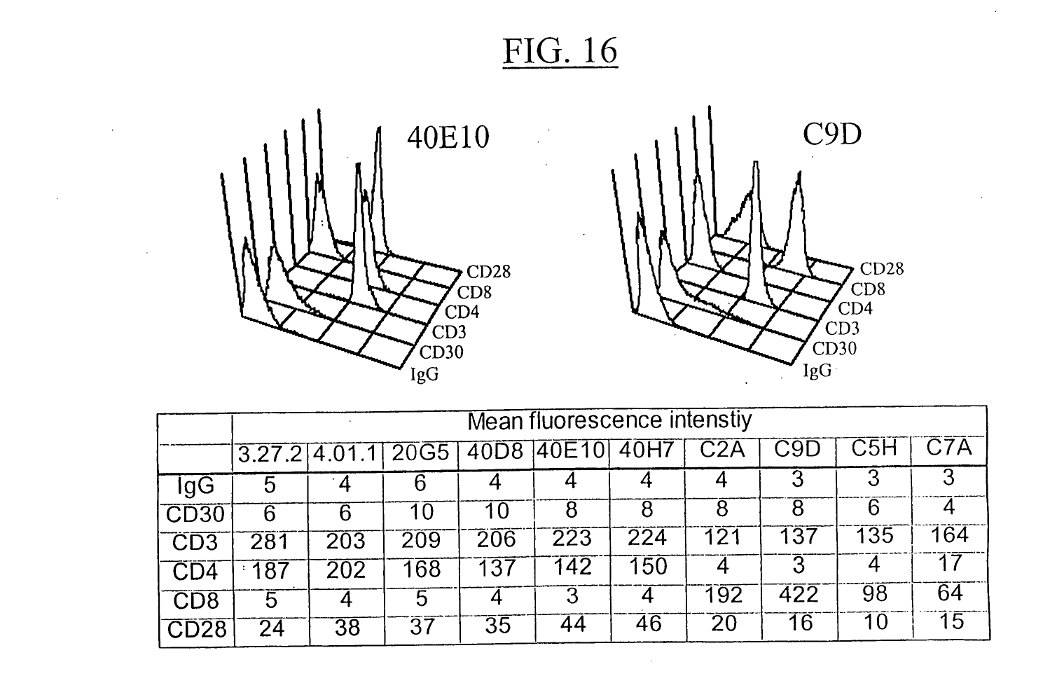

An alternative way to simulate effector lymphocytes in vitro is to generate T lymphocyte clones. FIG. 16 depicts a protocol for generating antigen non-specific helper (Th) and cytotoxic (Tc) T lymphocytes clones. PBMC were used as the starting material. CD4⁺ or CD8⁺ cells were enriched using the immuno-selection methods described in Examples 8 and 9. Two approaches were applied to stimulate and expand T lymphocytes. In the first approach, cells were stimulated with immobilized anti-CD3 plus soluble anti-CD28 and rhIL-2 (200 IU/ml) at limiting dilution in 96-well TC plates. Anti-CD3 was immobilized onto TC wells at one μg/ml as described in Example 6. Alternatively, cells were stimulated with phytohemagglutinin (Sigma, 1-2 μg/ml), 10,000 irradiated CESS cells (ATCC, 2700 rad irradiated), and rhIL-2 (200 IU/ml) at limiting dilution in 96-well plates. Additional supplements, including different combinations of cytokines and/or antibodies against cytokines can be used to skew the development of T lymphocyte clones to Th₁ or Th₂ effector cells.

Clones identified from the limiting dilution assays were further expanded by multiple rounds of re-stimulation; each round lasted for 10 to 12 days. During the second round of expansion, clones were stimulated with PHA (1-2 μg/ml), 1×10⁶ irradiated feeder cells (CESS), and rhIL-2 (200 IU/ml). In some experiments, anti-IL-12 (R&D Systems, 5 μg/ml) and LX (R&D Systems, 10 ng/ml) were supplemented to favor T lymphocyte clone development toward the Th₂ or Tc, subsets. During the subsequent rounds of re-stimulation a T lymphocytes:feeder cells ratio of 1:2 was used and IL-2 was supplemented at 200 IU/ml. Several panels of T cell clones were generated using these stimulation protocols, and they were subjected to further phenotypic and functional analysis.

### 16. EXAMPLE 11

### Phenotype of T Lymphocyte Clones

The surface phenotypes of 10 T lymphocyte clones determined by flow cytometry were depicted in FIG. 16. All 10 clones expressed high levels of CD3 and detectable levels of CD28. Six of the 10 clones expressed CD4, and they can be considered as T helper clones (Th). Three of the remaining four can be considered as cytotoxic T lymphocyte clones (Tc) as they expressed CD8. The last clone was CD4⁺/CD8⁺. Expression of CD30 was detectable on all 10 clones. The magnitudes of signals were relatively low, as the analysis was conducted on resting clones.

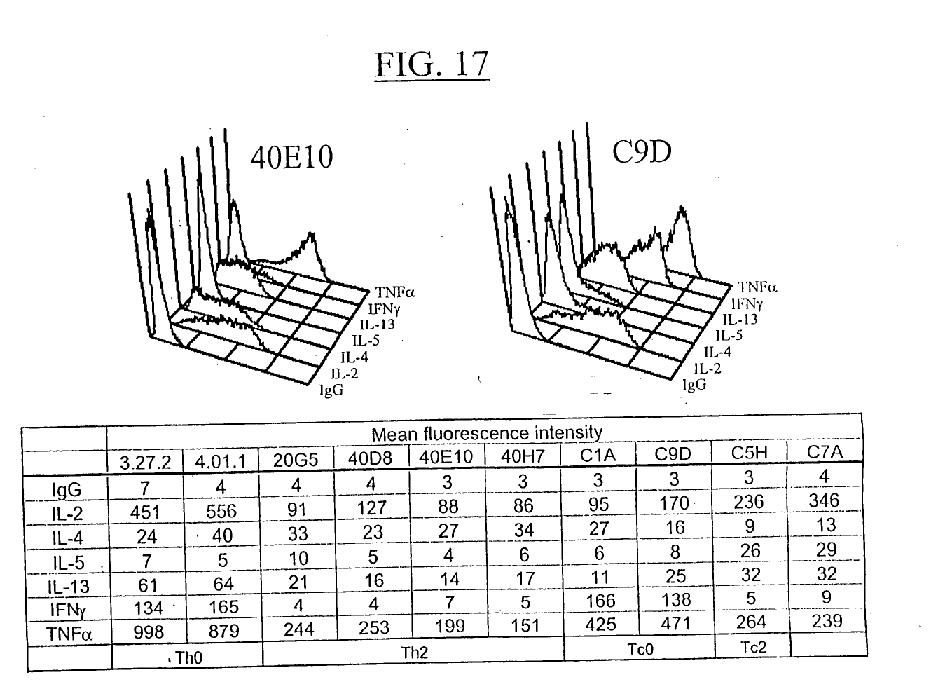

The T lymphocyte clones were also analyzed for cytokine expression (FIG. 17). Resting clones were stimulated for 4 hours at 37° C. with leukocyte activation cocktail (BD PharMingen). Golgi Plug (BD PharMingen) was added to enable accumulation of intracellular cytokine by blocking protein secretion. Cells were then washed and fixed with the Cytofix/Cytoperm Kit (BD PharMingen) according to the manufacturer's instruction. Intracellular cytokines were detected using various anti-cytokine antibodies (BD PharMingen), and stained cells were analyzed by flow cytometric analysis. Two of the Th clones expressed IL-4, IL-13, and IFNγ; this is consistent with a Tk profile. Four Th clones showed Th₂ profiles, i.e., they expressed IL-4, IL-5, or IL-13, but not IFNγ. Two Tc clones showed Tc₀ cytokine profile and one Tc clone showed Tc₂ profile. The CD4⁺/CD8⁺ clone expressed IL-4, IL-5, and IL-13, but not IFNγ.

### 17. EXAMPLE 12

### Re-Stimulation of T Lymphocyte Clones Induced Expression OF CD30

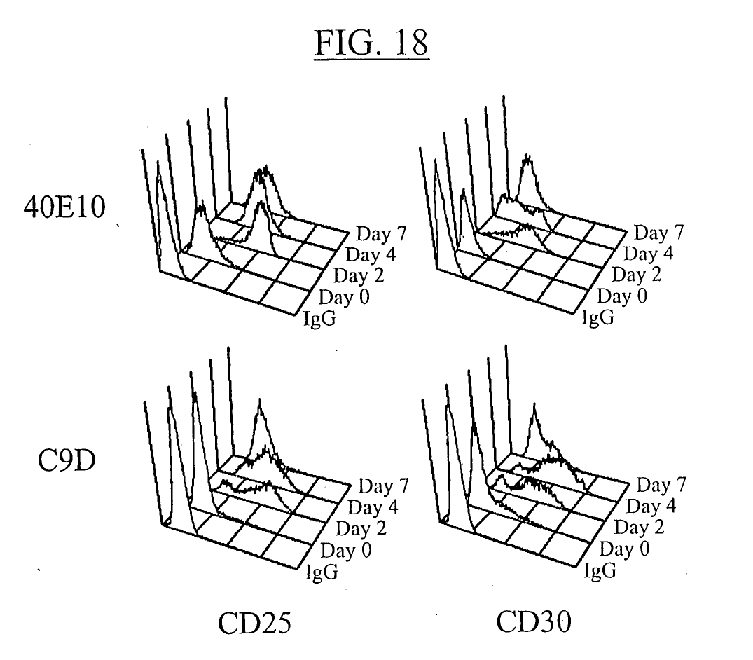

The expression of CD30 during re-activation of the T lymphocyte clones was examined. Resting T lymphocyte clones were stimulated with 1 or 2 μg/ml of PHA, irradiated CESS feeder cells at a T:CESS cell ratio of 1:2 to 1: 0, 200 IU/ml of IL-2. IL-4 at 20 ng/ml was also supplemented to the Th₂ and Tc₂ clones. The expression of CD25 and CD30 was monitored by flow cytometric analysis. FIG. 18 shows the results from one representative Th clone and one representative Tc clone. Extensive upregulation of CD25 was observed in all clones that peaked on day 2. This is indicative of T lymphocyte activation. Expression of CD25 gradually declined in the following days. The induction of CD30 expression paralleled that of CD25. The peak induction was also observed after 2 days of stimulation. Expression was still detectable on day 4 and it gradually declined to almost basal level by day 7. The other 8 clones examined also showed similar kinetics and magnitudes of CD25 and CD30 expression (data not shown). Activation-induced expression of both CD25 and CD30 has been a consistent feature of these T lymphocyte clones.

### 18. EXAMPLE 13

### cAC10 ADCS Inhibited the Proliferation of Activated T Lymphocyte Clones

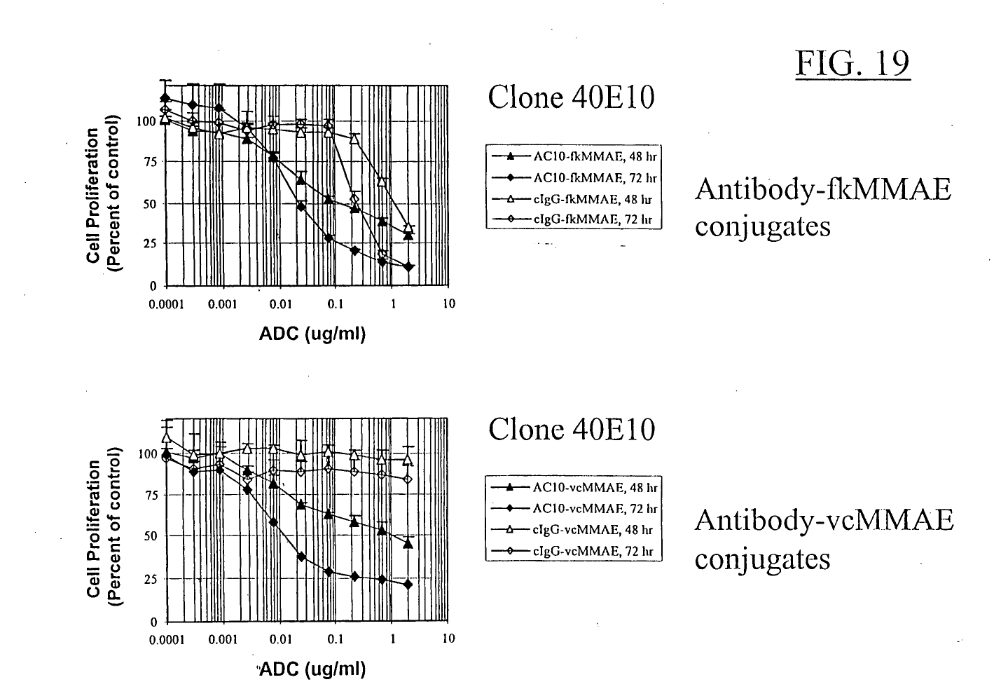

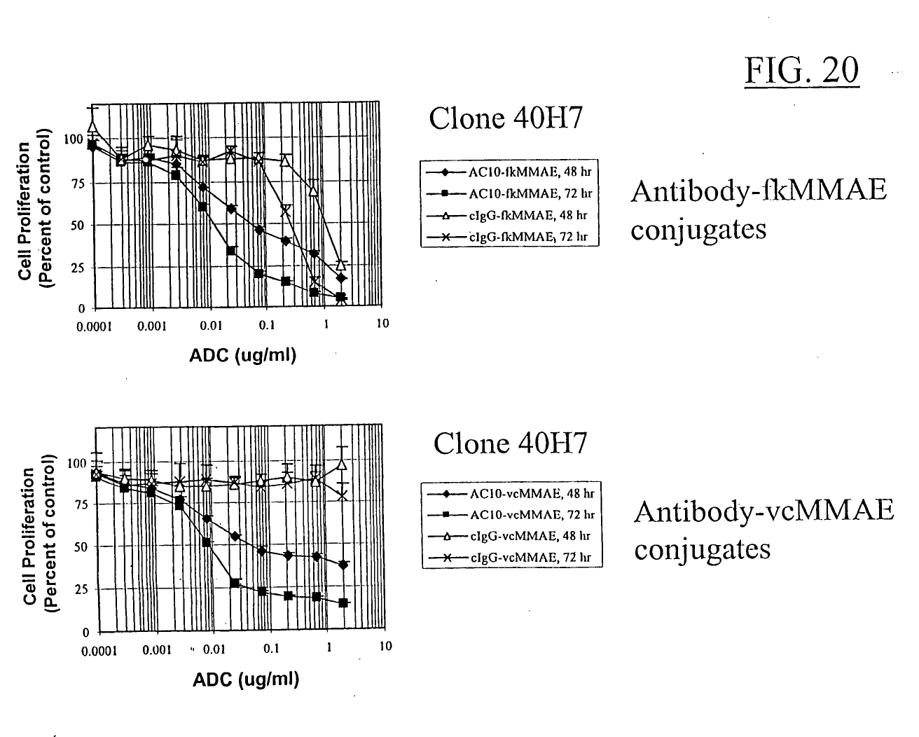

The responses of T lymphocyte clones to cAC10 ADCs were examined next. Resting T lymphocyte clones were activated to express CD30 as described in Example 12. After 2 days of activation, a portion of the cells was analyzed for CD30 expression by flow cytometry to confirm cellular activation and CD30 induction. The remaining cells were pelleted and resuspended in new medium containing 200 IU/ml of rIL-2 or 200 IU/ml of IL-2 and 10 ng/ml IL-4 for the Th₂ and Tc₂ clones. Cells were then plated out at 10,000 cells/well in a final volume of 200 μl of medium containing graded concentrations of cAC10 ADCs or the non-binding IgG ADCs as indicated in FIG. 19 and FIG. 20. Cells were incubated for an additional 48 or 72 hours with the last 16 hours pulsed with ³H-TdR to assess cellular DNA synthesis. Results for the responses of two Th₂ clones toward ADC treatment was shown in FIG. 19 and FIG. 20. Chimeric AC10 ADCs at concentrations higher than 0.01 μg/ml significantly inhibited the proliferation both clones. The control ADC cIgG-fkMMAE did not significantly inhibit proliferation at concentrations below 0.1 μg/ml, whereas for cIgG-vcMMAE concentrations as high as 2 μg/ml showed no growth inhibitory activity, confirming the antigen specificity of the cAC10 ADCs. An incubation of a total of 72 hours also resulted in much profound proliferation inhibition than a 48-hour incubation.

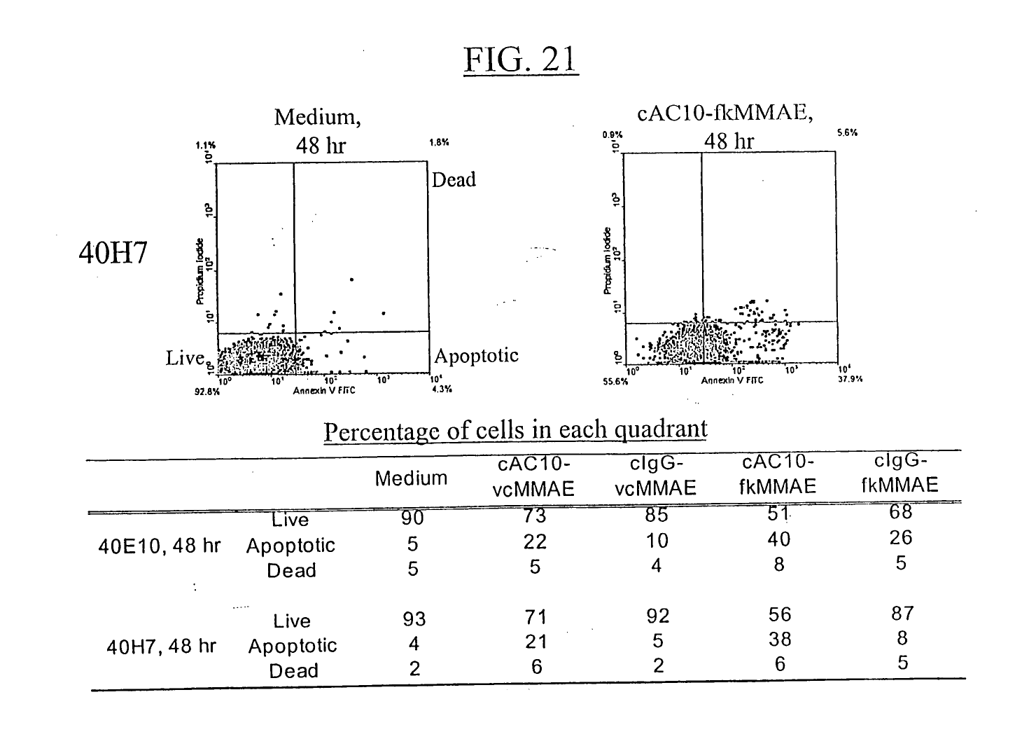

Annexin V binding and membrane permeability to PI described in Example 4 were then used to assess if the inhibition of proliferation effected by the cAC10 ADCs was accompanied by cell death (FIG. 21). After a 4S-hour incubation with 1 μg/ml of ADC, 27% of the cAC10-vcMMAE-treated cells was either apoptotic or dead, compared to 7-14% of the cIgG-fkMMAE-treated cells. For the cAC10-fkMMAE-treated cells, 44-48% were apoptotic or dead, compared to 13 and 31% of the cIG-fkMMAE-treated cells. These data confirmed that the cAC10 ADCs tested were cytotoxic to CD30⁺ T cell clones.

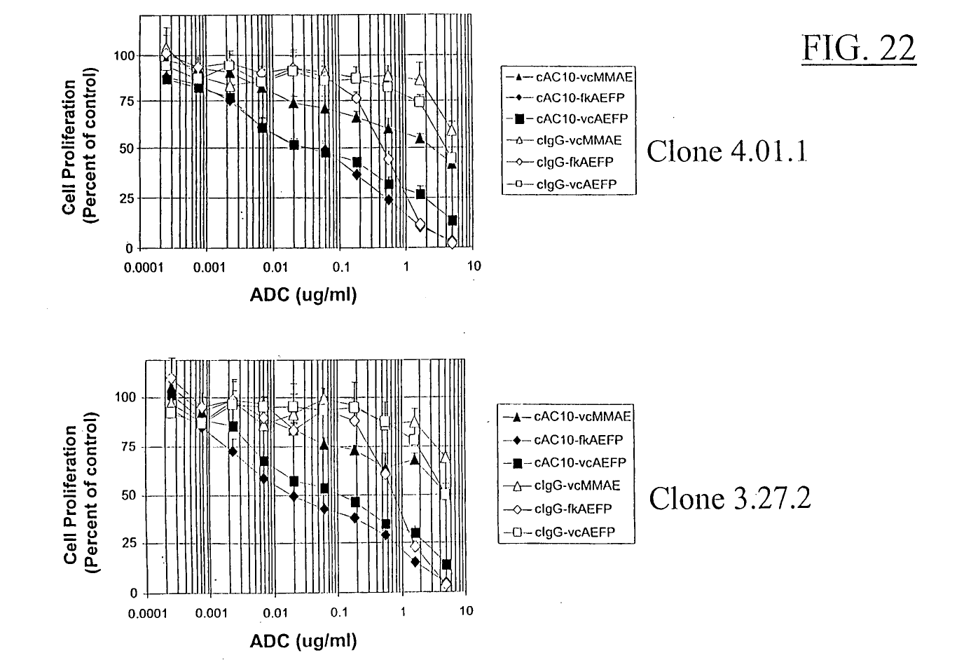

During the course of analyzing the effects of cAC10 ADCs on the proliferation of activated T lymphocyte clones, several clones were found to be relatively refractory to cAC10-vcMMAE treatment, e.g., clones 3.27.2 and 4.01.1 in FIG. 22. Similar to what was observed in memory and naïve T cells (FIG. 13), an AEFP conjugate of cAC10 showed strong inhibitory activity on the T lymphocyte clones. Thus, both cAC10-fkAEFP and cAC10-vcAEFP conjugates gave IC₅₀'s of 0.01 to 0.1 μg/ml, compared to >1 μg/ml from cAC10-vcMMAE. These data further confirm the utility of different cAC10 ADCs in targeted inhibition of proliferation in T lymphocytes.

### 19. EXAMPLE 14

### Antigen-Primed T Lymphocytes were More Sensitive to cAC10 ADCS

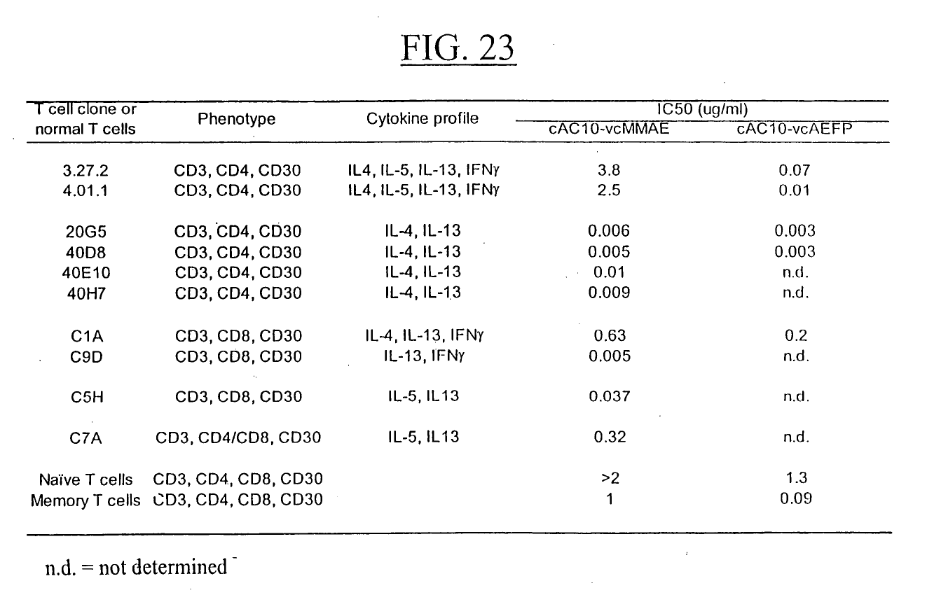

FIG. 23 summarizes the efficacies of cAC10-vcMMAE and cAC10-vcAEFP on inhibiting the proliferation of different types of T lymphocytes. The following trend was observed. First, naïve T cells appeared to be most refractory to both cAC10-vcMMAE and cAC10-vcAEFP when compared to memory T lymphocytes or T lymphocyte clones. Second, between the T lymphocyte clones and memory T cells, eight of the 10 T lymphocyte clones were found to be more sensitive to cAC10-vcMMAE compared to the memory T lymphocytes. These clones have been expanded through multiple rounds of T cell receptor and cytokine stimulation, similar to chronically stimulated effector T cells involved in inflammatory and autoimmune responses. Third, the proliferation of both CD4⁺ and CD8⁺ T lymphocytes clones was susceptible to cAC10 ADCs. Fourth, susceptibility to cAC10 ADC also did not appear to correlate to any particular T lymphocyte subsets as defined by their cytokine secretion profiles. Thus, Th₀, Th₂, and Tc₀ clones and one each of Tc₂ and CD4⁺/CD⁸⁺ clones were found to be sensitive to cAC10 ADCs. These results suggest that cAC10 ADCs could be used to target multiple T lymphocyte subsets including naïve or memory T lymphocytes, helper (CD4⁺) or cytotoxic (CD8⁺) lymphocytes, and effector lymphocytes secreting different combinations of cytokines. Moreover, since effector T lymphocytes participating in the pathogenesis of immune disorders are biologically more similar to the chronically activated T lymphocyte clones than to naïve T cells, cAC10 ADCs may be particularly suited for the targeted depletion of CD30⁻ effector T lymphocytes involved in the pathogenesis of autoimmune, inflammatory, and allergic responses.

### 20. Specific Embodiments Citation of References

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various references, including patent applications, patents, and scientific publications, are cited herein, the disclosures of each of which is incorporated herein by reference in its entirety.

1. A method for the treatment of an immunological disorder in a subject, wherein the immunological disorder is not cancer, comprising administering to the subject, in an amount effective for said treatment, a pharmaceutical composition comprising (a) a first antibody that (i) immunospecifically binds CD30 and (ii) exerts a cytostatic or cytotoxic effect on an activated lymphocyte; and (b) a pharmaceutically acceptable carrier.

2. The method of claim 1, wherein the first antibody is human, humanized or chimeric.

3. The method of claim 1, wherein the first antibody is multivalent.

4. The method of claim 1, wherein the first antibody competes for binding to CD30 with monoclonal antibodies AC10 or HeFi-1.

5. The method of claim 1, wherein the first antibody is capable of exerting the cytotoxic or cytostatic effect without conjugation to a cytotoxic agent.

6. The method of claim 1, wherein the first antibody is capable of exerting the cytotoxic or cytostatic effect in the absence of cells other than the activated lymphocyte.

7. The method of claim 1, wherein the first antibody is capable of exerting the cytotoxic or cytostatic effect as a monospecific antibody.

8. The method of claim 1, further comprising administering an agent that potentiates the cytostatic or cytotoxic effect of the first antibody.

9. The method of claim 1, further comprising administering a second antibody.

10. The method of claim 1, wherein the second antibody recognizes a second receptor or receptor complex expressed on activated lymphocytes.

11. The method of claim 10, wherein the second antibody enhances the cytostatic or cytotoxic effect of the first antibody .

12. The method of claim 11, wherein the second antibody enhances the cytostatic or cytotoxic effect of the first antibody by delivering a signal to the activated lymphocyte.

13. The method of claim 10 or 11, wherein the receptor or the receptor complex comprises an immunoglobulin gene superfamily member, a TNF receptor superfamily member, an integrin, a cytokine receptor, a chemokine receptor, a major histocompatibility protein, a lectin, or a complement control protein.

14. The method of claim 13, wherein the immunoglobulin superfamily member is CD2, CD3, CD4, CD8, CD19, CD22, CD28, CD79, CD90, CD152/CTLA-4, PD-1, or ICOS.

15. The method of claim 13, wherein the TNF receptor superfamily member is CD27, CD40, CD95/Fas, CD134/OX40, CD137/4-1BB, TNT-RL, TNFR-2, RANK, TACI, BCMA, osteoprotegerin, Apo2/TRAIL-R1, TRAIL-R-1, TRAIL-R3, TRAIL-R4, or APO-3.

16. The method of claim 13, wherein the integrin is CD11a, CD11b, CD 11c, CD18, CD29, CD41, CD49a, CD49b, CD49c, CD49d, CD49e, CD49f, CD103, or CD104.

17. The method of claim 13, wherein the lectin is C-type, S-type, or I-type lectin.

18. The method of claim 1, wherein the first antibody is a bispecific antibody.

19. The method of claim 18, wherein the wherein the bispecific antibody binds to CD30 and a second receptor or receptor complex expressed on activated lymphocytes.

20. The method of claim 19, wherein the portion of the bispecific antibody that binds to the second receptor or receptor complex enhances the cytostatic or cytotoxic effect of the portion of the bispecific antibody that binds to CD30.

21. The method of claim 20, wherein the binding of the bispecific antibody to the second receptor or receptor complex enhances the cytostatic or cytotoxic effect of the of the portion of the bispecific antibody that binds to CD30 by delivering a signal to the activated lymphocyte.

22. The method of claim 19 or 20, wherein the receptor or receptor complex comprises an immunoglobulin gene superfamily member, a TNF receptor superfamily member, an integrin, a cytokine receptor, a chemokine receptor, a major histocompatibility protein, a lectin, or a complement control protein.

23. The method of claim 22, wherein the immunoglobulin superfamily member is CD2, CD3, CD4, CD8, CD19, CD22, CD28, CD79, CD90, CD152/CTLA-4, PD-1, or ICOS.

24. The method of claim 22, wherein the TNF receptor superfamily member is CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, or APO-3.

25. The method of claim 22, wherein the integrin is CD11a, CD11b, CD11c, CD18, CD29, CD41, CD49a, CD49b, CD49c, CD49d, CD49e, CD49f, CD103, or CD104.

26. The method of claim 22, wherein the lectin is C-type, S-type, or I-type lectin.

27. The method of claim 1, further comprising administering a ligand that binds to a receptor or receptor complex expressed on activated lymphocytes.

28. The method of claim 27, wherein the ligand enhances the cytostatic or cytotoxic effect of the first antibody.

29. The method of claim 28, wherein the ligand enhances the cytostatic or cytotoxic effect of the first antibody by delivering a signal to the activated lymphocyte.

30. The method of claim 27 or 28, wherein the receptor or receptor complex comprises an immunoglobulin gene superfamily member, a TNF receptor superfamily member, an integrin, a cytokine receptor, a chemokine receptor, a major histocompatibility protein, a lectin, or a complement control protein.

31. The method of claim 30, wherein the immunoglobulin superfamily member is CD2, CD3, CD4, CD8, CD19, CD22, CD28, CD79, CD90, CD152/CTLA-4, PD-1, or ICOS.

32. The method of claim 30, wherein the TNF receptor superfamily member is CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, or APO-3.

33. The method of claim 30, wherein the integrin is CD11a, CD11b, CD11c, CD18, CD29, CD41, CD49a, CD49b, CD49c, CD49d, CD49e, CD49f, CD103, or CD104.

34. The method of claim 30, wherein the lectin is C-type, S-type, or I-type lectin.

35. The method of claim 1, wherein the first antibody is a fusion protein comprising the amino acid sequence of a second protein that is not an antibody.

36. The method of claim 35, wherein the second protein confers multivalent binding properties to the first antibody.

37. The method of claim 1, 9, 10, 18, or 27, further comprising administering an immunosuppressive agent.

38. The method of claim 37, wherein the immunosuppressive agent is gancyclovir, etanercept, cyclosporine, tacrolimus, or rapamycin.

39. The method of claim 37, wherein the immunosuppressive agent is an alkylating agent.

40. The method of claim 39, wherein the alkylating agent is cyclophosphamide.

41. The method of claim 37, wherein the immunosuppressive agent is an antimetabolite.

42. The method of claim 41, wherein the antimetabolite is a purine antagonist.

43. The method of claim 42, wherein the purine antagonist is azathioprine, or mycophenolate mofetil.

44. The method of claim 41, wherein the antimetabolite is a dihydrofolate reductase inhibitor.

45. The method of claim 44, wherein the dihydrofolate reductase inhibitor is methotrexate.

46. The method of claim 37, wherein the immunosuppressive agent is a glucocorticoid.

47. The method of claim 46, wherein the glucocorticoid is cortisol or aldosterone.

48. The method of claim 37, wherein the immunosuppressive agent is a glucocorticoid analogue.

49. The method of claim 48, wherein the glucocorticoid analogue is prednisone or dexamethasone.

50. The method of claim 37, wherein the immunosuppressive agent is an anti-inflammatory agent.

51. The method of claim 50, wherein the anti-inflammatory agent is a cyclooxygenase inhibitor, a 5-lipoxygenase inhibitor, or a leukotriene receptor antagonist.

52. The method of claim 1, wherein the first antibody is conjugated to a cytotoxic agent.

53. The method of claim 52, wherein the cytotoxic agent is selected from the group consisting of an enediyne, a lexitropsin, a duocarmycin, a taxane, a puromycin, a dolastatin, a maytansinoid, a DNA minor groove binding agent, a DNA minor groove alkylating agent, and a vinca alkaloid.

54. The method of claim 52, wherein the cytotoxic agent is paclitaxel, docetaxel. CC-1065, SN-38, topotecan, morpholino-doxorubicin, rhizoxin, cyanomorpholino-doxorubicin, dolastatin-10, echinomycin, combretastatin, calicheamicin, maytansine, DM-1, auristatin E, AEB. AEVB, AEFP, MMAE, or netropsin.

55. The method of claim 52, wherein the cytotoxic agent is an anti-tubulin agent.

56. The method of claim 55, wherein the cytotoxic agent is a vinca alkaloid, a podophyllotoxin, a taxane, a baccatin derivative, a cryptophysin, a maytansinoid, a combretastatin, or a dolastatin.

57. The method of claim 55, wherein the cytotoxic agent is vincristine, vinblastine, vindesine, vinorelbine, VP-16, camptothecin, paclitaxel, docetaxel, epithilone A, epithilone B, nocodazole, colchicine, colcimid, estramustine, cemadotin, discodermolide, maytansine, DM-1, auristatin E, AEB, AEVB, AEFP, MMAE, or eleutherobin.

58. The method of claim 52, wherein the cytotoxic agent is MMAE.

59. The method of claim 52, wherein the cytotoxic agent is AEFP.

60. The method of claim 52, wherein the first antibody is conjugated to the cytotoxic agent via a peptide linker.

61. The method of claim 52, wherein the first antibody is conjugated to the cytotoxic agent via a val-cit linker or a phe-lys linker.

62. The method of claim 52, wherein the first antibody is conjugated to the cytotoxic agent via a hydrazone-linker, or a disulfide-linker.

63. The method of claim 52, wherein the conjugate is cAC10-val-cit-MMAE.

64. The method of claim 52, wherein the conjugate is cAC10-val-cit-AEFP.

65. The method of claim 52, wherein the first antibody is conjugated to the cytotoxic agent via a linker that is hydrolyzable at a pH of less than 5.5.

66. The method of claim 65, wherein the linker is hydrolyzable at a pH of less than 5.0.

67. The method of claim 65, wherein the linker is a hydrazone linker or a disulfide linker.

68. The method of claim 52, wherein the first antibody is conjugated to the cytotoxic agent via a linker, wherein the linker is cleavable by a protease.

69. The method of claim 52, wherein the first antibody is conjugated to the cytotoxic agent via a peptide linker, and wherein the linker is cleavable by a protease.

70. The method of claim 68, wherein the protease is a membrane-associated protease.

71. The method of claim 68, wherein the protease is an intracellular protease.

72. The method of claim 68, wherein the protease is an endosomal protease.

73. The method of claim 68, wherein the protease is a lysosomal protease.

74. The method of claim 52, wherein the first antibody is a monoclonal antibody, a chimeric antibody, a human antibody, a humanized antibody, a glycosylated antibody, a multispecific antibody, a single-chain antibody, a Fab fragment, a F(ab′) fragment, a F(ab′)₂ fragment, a Fd, a single-chain Fv, a disulfide-linked Fv, a fragment comprising a V_{L} domain, a polypeptide that binds specifically to CD30, or a fragment comprising a V_{H} domain.

75. The method of claim 1, wherein the first antibody is conjugated to a immunosuppressive agent.

76. The method of claim 75, wherein the immunosuppressive agent is gancyclovir, etanercept, cyclosporine, tacrolimus, or rapamycin.

77. The method of claim 75, wherein the immunosuppressive agent is an alkylating agent.

78. The method of claim 77, wherein the alkylating agent is cyclophosphamide.

79. The method of claim 75, wherein the immunosuppressive agent is an antimetabolite.

80. The method of claim 79, wherein the antimetabolite is a purine antagonist.

81. The method of claim 80, wherein the purine antagonist is azathioprine, or mycophenolate mofetil.

82. The method of claim 79, wherein the antimetabolite is a dihydrofolate reductase inhibitor.

83. The method of claim 82, wherein the dihydrofolate reductase inhibitor is methotrexate.

84. The method of claim 75, wherein the immunosuppressive agent is a glucocorticoid.

85. The method of claim 84, wherein the glucocorticoid is cortisol or aldosterone.

86. The method of claim 75, wherein the immunosuppressive agent is a glucocorticoid analogue.

87. The method of claim 86, wherein the glucocorticoid analogue is prednisone or dexamethasone.

88. The method of claim 75, wherein the immunosuppressive agent is an anti-inflammatory agent.

89. The method of claim 88, wherein the anti-inflammatory agent is a cyclooxygenase inhibitor, a 5-lipoxygenase inhibitor, or a leukotriene receptor antagonist.

90. The method of claim 1, wherein the immunological disorder is a Th₂-lymphocyte related disorder.

91. The method of claim 90, wherein the immunological disorder is atopic dermatitis, systemic lupus erythematosus, atopic asthma, rhinoconjunctivitis, allergic rhinitis, Omenn's syndrome, systemic sclerosis, or chronic graft versus host disease.

92. The method of claim 1, wherein the immunological disorder is a Th₁ lymphocyte-related disorder.

93. The method of claim 92, wherein the immunological disorder is rheumatoid arthritis, multiple sclerosis, psoriasis, Sjorgren's syndrome, Hashimoto's thyroiditis, Grave's disease, primary biliary cirrhosis, Wegener's granulomatosis, tuberculosis, or acute graft versus host disease.

94. The method of claim 1, wherein the immunological disorder is due to viral infection.

95. The method of claim 94, wherein the viral infection involves the Epstein-Barr virus, human immunodeficiency virus, human T leukemia virus, hepatitis B virus, or measles virus.

96. The method of claim 1, wherein the immunological disorder is an activated B lymphocyte-related disorder.

97. A method for the treatment of an immunological disorder in a subject, wherein the immunological disorder is not cancer, comprising administering to the subject, in an amount effective for said treatment, a pharmaceutical composition comprising (a) a first antibody that (i) immunospecifically binds CD30 and (ii) induces CD30 signaling in a lymphocyte; and (b) a pharmaceutically acceptable carrier.

98. The method of claim 97, wherein the first antibody is human, humanized or chimeric.

99. The method of claim 97, wherein the first antibody is multivalent.

100. The method of claim 97, wherein the first antibody competes for binding to CD30 with monoclonal antibodies AC10 or HeFi-1.

101. The method of claim 97, wherein the first antibody is capable of inducing CD30 signaling in the absence of cells other than the lymphocyte.

102. The method of claim 97, wherein the first antibody is capable of inducing CD30 signaling as a monospecific antibody.

103. The method of claim 97, further comprising administering a second antibody.

104. The method of claim 97, wherein the second antibody recognizes a second receptor or receptor complex expressed on activated lymphocytes.

105. The method of claim 104, wherein the receptor or the receptor complex comprises an immunoglobulin gene superfamily member, a TNF receptor superfamily member, an integrin, a cytokine receptor, a chemokine receptor, a major histocompatibility protein, a lectin, or a complement control protein.

106. The method of claim 105, wherein the immunoglobulin superfamily member is CD2, CD3, CD4, CD8, CD19, CD22, CD28, CD79, CD90, CD152/CTLA-4, PD-1, or ICOS.

107. The method of claim 105, wherein the TNF receptor superfamily member is CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, or APO-3.

108. The method of claim 105, wherein the integrin is CD11a, CD11b, CD11c, CD18, CD29, CD41, CD49a, CD49b, CD49c, CD49d, CD49e, CD49f, CD103, or CD104.

109. The method of claim 105, wherein the lectin is C-type, S-type, or I-type lectin.

110. The method of claim 97, wherein the first antibody is a bispecific antibody.

111. The method of claim 110, wherein the wherein the bispecific antibody binds to CD30 and a second receptor or receptor complex expressed on activated lymphocytes.

112. The method of claim 111, wherein the receptor or receptor complex comprises an immunoglobulin gene superfamily member, a TNF receptor superfamily member, an integrin, a cytokine receptor, a chemokine receptor, a major histocompatibility protein, a lectin, or a complement control protein.

113. The method of claim 112, wherein the immunoglobulin superfamily member is CD2, CD3, CD4, CD8, CD19, CD22, CD28, CD79, CD90, CD152/CTLA4, PD-1, or ICOS.

114. The method of claim 112, wherein the TNF receptor superfamily member is CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, or APO-3.

115. The method of claim 112, wherein the integrin is CD11a, CD11b, CD11c, CD18, CD29, CD41, CD49a, CD49b, CD49c, CD49d, CD49e, CD49f, CD103, or CD104.

116. The method of claim 112, wherein the lectin is C-type, S-type, or I-type lectin.

117. The method of claim 97, further comprising administering a ligand that binds to a receptor or receptor complex expressed on activated lymphocytes.

118. The method of claim 117, wherein the receptor or receptor complex comprises an immunoglobulin gene superfamily member, a TNF receptor superfamily member, an integrin, a cytokine receptor, a chemokine receptor, a major histocompatibility protein, a lectin, or a complement control protein.

119. The method of claim 118, wherein the immunoglobulin superfamily member is CD2, CD3, CD4, CD8, CD19, CD22, CD28, CD79, CD90, CD152/CTLA-4, PD-1, or ICOS.

120. The method of claim 118, wherein the TNF receptor superfamily member is CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, or APO-3.

121. The method of claim 118, wherein the integrin is CD11a, CD11b, CD11c, CD18, CD29, CD41, CD49a, CD49b, CD49c, CD49d, CD49e, CD49f, CD103, or CD104.

122. The method of claim 118, wherein the lectin is C-type, S-type, or I-type lectin.

123. The method of claim 97, wherein the first antibody is a fusion protein comprising the amino acid sequence of a second protein that is not an antibody.

124. The method of claim 123, wherein the second protein confers multivalent binding properties to the first antibody.

125. The method of claim 97, 103, 104, 110, or 111, further comprising administering an immunosuppressive agent.

126. The method of claim 125, wherein the immunosuppressive agent is gancyclovir, etanercept, cyclosporine, tacrolimus, or rapamycin.

127. The method of claim 125, wherein the immunosuppressive agent is an alkylating agent.

128. The method of claim 127, wherein the alkylating agent is cyclophosphamide.

129. The method of claim 125, wherein the immunosuppressive agent is an antimetabolite.

130. The method of claim 129, wherein the antimetabolite is a purine antagonist.

131. The method of claim 130, wherein the purine antagonist is azathioprine, or mycophenolate mofetil.

132. The method of claim 129, wherein the antimetabolite is a dihydrofolate reductase inhibitor.

133. The method of claim 132, wherein the dihydrofolate reductase inhibitor is methotrexate.

134. The method of claim 125, wherein the immunosuppressive agent is a glucocorticoid.

135. The method of claim 134, wherein the glucocorticoid is cortisol or aldosterone.

136. The method of claim 125, wherein the immunosuppressive agent is a glucocorticoid analogue.

137. The method of claim 136, wherein the glucocorticoid analogue is prednisone or dexamethasone.

138. The method of claim 125, wherein the immunosuppressive agent is an anti-inflammatory agent.

139. The method of claim 138, wherein the anti-inflammatory agent is a cyclooxygenase inhibitor, a 5-lipoxygenase inhibitor, or a leukotriene receptor antagonist.

140. The method of claim 97, wherein the first antibody is conjugated to a cytotoxic agent.

141. The method of claim 140, wherein the cytotoxic agent is selected from the group consisting of an enediyne, a lexitropsin, a duocarmycin, a taxane, a puromycin, a dolastatin, a maytansinoid, a DNA minor groove binding agent, a DNA minor groove alkylating agent, and a vinca alkaloid.

142. The method of claim 140, wherein the cytotoxic agent is paclitaxel, docetaxel, CC-1065, SN-38, topotecan, morpholino-doxorubicin, rhizoxin, cyanomorpholino-doxorubicin, dolastatin-10, echinomycin, combretastatin, calicheamicin, maytansine, DM-1, auristatin E, AEB, AEVB, AEFP, MMAE, or netropsin.

143. The method of claim 140, wherein the cytotoxic agent is an anti-tubulin agent.

144. The method of claim 143, wherein the cytotoxic agent is a vinca alkaloid, a podophyllotoxin, a taxane, a baccatin derivative, a cryptophysin, a maytansinoid, a combretastatin, or a dolastatin.

145. The method of claim 143, wherein the cytotoxic agent is vincristine, vinblastine, vindesine, vinorelbine, VP-16, camptothecin, paclitaxel, docetaxel, epithilone A, epithilone B, nocodazole, colchicine, colcimid, estramustine, cemadotin, discodermolide, maytansine, DM-1, auristatin E, AEB, AEVB, AEFP, MMAE, or eleutherobin.

146. The method of claim 140, wherein the cytotoxic agent is MMAE.

147. The method of claim 140, wherein the cytotoxic agent is AEFP.

148. The method of claim 140, wherein the first antibody is conjugated to the cytotoxic agent via a peptide linker.

149. The method of claim 140, wherein the first antibody is conjugated to the cytotoxic agent via a val-cit linker or a phe-lys linker.

150. The method of claim 140, wherein the first antibody is conjugated to the cytotoxic agent via a hydrazone-linker, or a disulfide-linker.

151. The method of claim 140, wherein the first antibody is conjugated to the cytotoxic agent via a linker that is hydrolyzable at a pH of less than 5.5.

152. The method of claim 151, wherein the linker is hydrolyzable at a pH of less than 5.0.

153. The method of claim 151, wherein the linker is a hydrazone linker or a disulfide linker.

154. The method of claim 140, wherein the first antibody is conjugated to the cytotoxic agent via a linker, wherein the linker is cleavable by a protease.

155. The method of claim 140, wherein the first antibody is conjugated to the cytotoxic agent via a peptide linker, and wherein the linker is cleavable by a protease.

156. The method of claim 154, wherein the protease is a membrane-associated protease.

157. The method of claim 154, wherein the protease is an intracellular protease.

158. The method of claim 154, wherein the protease is an endosomal protease.

159. The method of claim 154, wherein the protease is a lysosomal protease.

160. The method of claim 140, wherein the first antibody is a monoclonal antibody, a chimeric antibody, a human antibody, a humanized antibody, a glycosylated antibody, a multispecific antibody, a single-chain antibody, a Fab fragment, a F(ab′) fragment, a F(ab′)₂ fragment, a Fd, a single-chain Fv, a disulfide-linked Fv, a fragment comprising a V_{L} domain, a polypeptide that binds specifically to CD30, or a fragment comprising a V_{H} domain.

161. The method of claim 97, wherein the first antibody is conjugated to a immunosuppressive agent.

162. The method of claim 161, wherein the immunosuppressive agent is gancyclovir, etanercept, cyclosporine, tacrolimus, or rapamycin.

163. The method of claim 161 wherein the immunosuppressive agent is an alkylating agent.

164. The method of claim 163, wherein the alkylating agent is cyclophosphamide.

165. The method of claim 161, wherein the immunosuppressive agent is an antimetabolite.

166. The method of claim 165, wherein the antimetabolite is a purine antagonist.

167. The method of claim 166, wherein the purine antagonist is azathioprine, or mycophenolate mofetil.

168. The method of claim 165, wherein the antimetabolite is a dihydrofolate reductase inhibitor.

169. The method of claim 168, wherein the dihydrofolate reductase inhibitor is methotrexate.

170. The method of claim 161, wherein the immunosuppressive agent is a glucocorticoid.

171. The method of claim 170, wherein the glucocorticoid is cortisol or aldosterone.

172. The method of claim 161, wherein the immunosuppressive agent is a glucocorticoid analogue.

173. The method of claim 172, wherein the glucocorticoid analogue is prednisone or dexamethasone.

174. The method of claim 161, wherein the immunosuppressive agent is an anti-inflammatory agent.

175. The method of claim 174, wherein the anti-inflammatory; agent is a cyclooxygenase inhibitor, a 5-lipoxygenase inhibitor, or a leukotriene receptor antagonist.

176. The method of claim 97, wherein the immunological disorder is a Th₂-lymphocyte related disorder.

177. The method of claim 176, wherein the immunological disorder is atopic dermatitis, systemic lupus erythematosus, atopic asthma, rhinoconjunctivitis, allergic rhinitis, Omenn's syndrome, systemic sclerosis, or chronic graft versus host disease.

178. The method of claim 97, wherein the immunological disorder is a Th₁ lymphocyte-related disorder.

179. The method of claim 178, wherein the immunological disorder is rheumatoid arthritis, multiple sclerosis, psoriasis, Sjorgren's syndrome, Hashimoto's thyroiditis, Grave's disease, primary biliary cirrhosis, Wegener's granulomatosis, tuberculosis, or acute graft versus host disease.

180. The method of claim 97, wherein the immunological disorder is due to viral infection.

181. The method of claim 180, wherein the viral infection involves the Epstein-Barr virus, human immunodeficiency virus, human T leukemia virus, hepatitis B virus, or measles virus.

182. The method of claim 97, wherein the immunological disorder is an activated B lymphocyte-related disorder.

183. A method for the treatment of an immunological disorder in a subject, wherein the immunological disorder is not cancer, comprising administering to the subject, in an amount effective for said treatment, a pharmaceutical composition comprising (a) an antibody that (i) immunospecifically binds CD30 and (ii) competes for binding to CD30 with monoclonal antibody AC10 or HeFi-1; and (b) a pharmaceutically acceptable carrier.

184. A method for the treatment of an immunological disorder in a subject, wherein the immunological disorder is not cancer, comprising administering to the subject, in an amount effective for said treatment, a pharmaceutical composition comprising (a) an antibody that (i) immunospecifically binds CD30 and (ii) comprises SEQ ID NO:2; and (b) a pharmaceutically acceptable carrier.

185. A method for the treatment of an immunological disorder in a subject, wherein the immunological disorder is not cancer, comprising administering to the subject, in an amount effective for said treatment, a pharmaceutical composition comprising (a) an antibody that (i) immunospecifically binds CD30 and (ii) comprises one, two or all of: SEQ ID NO:4, SEQ ID NO:6 and SEQ ID NO:8; and (b) a pharmaceutically acceptable carrier.

186. A method for the treatment of an immunological disorder in a subject, wherein the immunological disorder is not cancer, comprising administering to the subject, in an amount effective for said treatment, a pharmaceutical composition comprising (a) an antibody that (i) immunospecifically binds CD30 and (ii) comprises SEQ ID NO: 18; and (b) a pharmaceutically acceptable carrier.

187. A method for the treatment of an immunological disorder in a subject, wherein the immunological disorder is not cancer, comprising administering to the subject, in an amount effective for said treatment, a pharmaceutical composition comprising (a) an antibody that (i) immunospecifically binds CD30 and (ii) comprises one, two or all of: SEQ ID NO:20, SEQ ID NO:22 and SEQ ID NO:24; and (b) a pharmaceutically acceptable carrier.

188. A method for the treatment of an immunological disorder in a subject, wherein the immunological disorder is not cancer, comprising administering to the subject, in an amount effective for said treatment, a pharmaceutical composition comprising (a) an antibody that (i) immunospecifically binds CD30 and (ii) competes for binding to CD30 with monoclonal antibody AC10 or HeFi-1, wherein said antibody is conjugated to a cytotoxic agent; and (b) a pharmaceutically acceptable carrier.

189. The method of claim 188, wherein the cytotoxic agent is selected from the group consisting of an enediyne, a lexitropsin, a duocarmycin, a taxane, a puromycin, a dolastatin, a maytansinoid, a DNA minor groove binding agent, a DNA minor groove alkylating agent, and a vinca alkaloid.

190. The method of claim 188, wherein the cytotoxic agent is paclitaxel, docetaxel, CC-1065, SN-38, topotecan, morpholino-doxorubicin, rhizoxin, cyanomorpholino-doxorubicin, dolastatin-10, echinomycin, combretastatin, calicheamicin, maytansine, DM-1, auristatin E, AEB, AEVB, AEFP, MMAE, or netropsin.

191. The method of claim 188, wherein the cytotoxic agent is an anti-tubulin agent.

192. The method of claim 191, wherein the cytotoxic agent is a vinca alkaloid, a podophyllotoxin, a taxane, a baccatin derivative, a cryptophysin, a maytansinoid, a combretastatin, or a dolastatin.

193. The method of claim 191, wherein the cytotoxic agent is vincristine, vinblastine, vindesine, vinorelbine, VP-16, camptothecin, paclitaxel, docetaxel, epithilone A, epithilone B, nocodazole, colchicine, colcimid, estramustine, cemadotin, discodermolide, maytansine, DM-1, auristatin E, AEB, AEVB, AEFP, MMAE, or eleutherobin.

194. The method of claim 188, wherein the cytotoxic agent is MMAE.

195. The method of claim 188, wherein the cytotoxic agent is AEFP.

196. The method of claim 188, wherein the first antibody is conjugated to the cytotoxic agent via a peptide linker.

197. The method of claim 188, wherein the first antibody is conjugated to the cytotoxic agent via a val-cit linker or a phe-lys linker.

198. The method of claim 18S, wherein the first antibody is conjugated to the cytotoxic agent via a hydrazone-linker, or a disulfide-linker.

199. The method of claim 188, wherein the first antibody is conjugated to the cytotoxic agent via a linker that is hydrolyzable at a pH of less than 5.5.

200. The method of claim 199, wherein the linker is hydrolyzable at a pH of less than 5.0.

201. The method of claim 199, wherein the linker is a hydrazone linker or a disulfide linker.

202. The method of claim 18S, wherein the first antibody is conjugated to the cytotoxic agent via a linker, wherein the linker is cleavable by a protease.

203. The method of claim 188 wherein the first antibody is conjugated to the cytotoxic agent via a peptide linker, and wherein the linker is cleavable by a protease.

204. The method of claim 202, wherein the protease is a membrane-associated protease.

205. The method of claim 202, wherein the protease is an intracellular protease.

206. The method of claim 202, wherein the protease is an endosomal protease.

207. The method of claim 202, wherein the protease is a lysosomal protease.

208. The method of claim 188, wherein the first antibody is a monoclonal antibody, a chimeric antibody, a human antibody, a humanized antibody, a glycosylated antibody, a multi specific antibody, a single-chain antibody, a Fab fragment, a F(ab′) fragment, a F(ab′)₂ fragment, a Fd, a single-chain Fv, a disulfide-linked Fv, a fragment comprising a VL domain, a polypeptide that binds specifically to CD30, or a fragment comprising a VH domain.

209. A method for the treatment of an immunological disorder in a subject, wherein the immunological disorder is not cancer, comprising administering to the subject, in an amount effective for said treatment, a pharmaceutical composition comprising (a) cAC10-val-cit-MMAE; and (b) a pharmaceutically acceptable carrier.

210. A method for the treatment of an immunological disorder in a subject, wherein the immunological disorder is not cancer, comprising administering to the subject, in an amount effective for said treatment, a pharmaceutical composition comprising (a) cAC10-val-cit-AEFP; and (b) a pharmaceutically acceptable carrier.


SEQUENCE LISTING (33 sequences)

SEQ ID NO: 1 - DNA, 351 bp, Mus musculus
  CDS (1)...(351)
cagatccagctgcagcagtctggacctgaggtggtgaagcctggggcttcagtgaagatatcctgcaaggcttctggctacaccttcactgactactatataacctgggtgaagcagaagcctggacagggacttgagtggattggatggatttatcctggaagcggtaatactaagtacaatgagaagttcaagggcaaggccacattgactgtagacacatcctccagcacagccttcatgcagctcagcagcctgacatctgaggacactgctgtctatttctgtgcgaactatggtaactactggtttgcttactggggccaagggactcaggtcactgtctctgca

SEQ ID NO: 2 - protein, 117 aa, Mus musculus
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 3 - DNA, 15 bp, Mus musculus
gactactatataacc

SEQ ID NO: 4 - protein, 5 aa, Mus musculus
DYYIT

SEQ ID NO: 5 - DNA, 51 bp, Mus musculus
tggatttatcctggaagcggtaatactaagtacaatgagaagttcaagggc

SEQ ID NO: 6 - protein, 17 aa, Mus musculus
WIYPGSGNTKYNEKFKG

SEQ ID NO: 7 - DNA, 24 bp, Mus musculus
tatggtaactactggtttgcttac

SEQ ID NO: 8 - protein, 8 aa, Mus musculus
YGNYWFAY

SEQ ID NO: 9 - DNA, 333 bp, Mus musculus
  CDS (1)...(333)
gacattgtgctgacccaatctccagcttctttggctgtgtctctagggcagagggccaccatctcctgcaaggccagccaaagtgttgattttgatggtgatagttatatgaactggtaccaacagaaaccaggacagccacccaaagtcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtggacgttcggtggaggcaccaagctggaaatcaaa

SEQ ID NO: 10 - protein, 111 aa, Mus musculus
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 11 - DNA, 45 bp, Mus musculus
aaggccagccaaagtgttgattttgatggtgatagttatatgaac

SEQ ID NO: 12 - protein, 15 aa, Mus musculus
KASQSVDFDGDSYMN

SEQ ID NO: 13 - DNA, 21 bp, Mus musculus
gctgcatccaatctagaatct

SEQ ID NO: 14 - protein, 7 aa, Mus musculus
AASNLES

SEQ ID NO: 15 - DNA, 27 bp, Mus musculus
cagcaaagtaatgaggatccgtggacg

SEQ ID NO: 16 - protein, 9 aa, Mus musculus
QQSNEDPWT

SEQ ID NO: 17 - DNA, 375 bp, Mus musculus
  CDS (1)...(375)
gaggtgaagctggtggagtctggaggaggcttggtacagcctgggggttctctgagactctcctgtgcaacttctgggttcaccttcagtgattactatatgaactgggtccgccagcctccaggaaaggctcttgagtggttgggttttattagaaacaaagctaatggttacacaacagagttcagtgcatctgtgatgggtcggttcaccatctccagagatgattcccaaagcatcctctatcttcagatgaacaccctgagagctgaggacagtgccacttattactgtgcaagagatcccccctatggtaacccccattattatgctatggactactggggtcaaggaacctcagtcaccgtctcctca

SEQ ID NO: 18 - protein, 125 aa, Mus musculus
EVKLVESGGGLVQPGGSLRLSCATSGFTFSDYYMNWVRQPPGKALEWLGFIRNKANGYTTEFSASVMGRFTISRDDSQSILYLQMNTLRAEDSATYYCARDPPYGNPHYYAMDYWGQGTSVTVSS

SEQ ID NO: 19 - DNA, 15 bp, Mus musculus
gattactatatgaac

SEQ ID NO: 20 - protein, 5 aa, Mus musculus
DYYMN

SEQ ID NO: 21 - DNA, 57 bp, Mus musculus
tttattagaaacaaagctaatggttacacaacagagttcagtgcatctgtgatgggt

SEQ ID NO: 22 - protein, 19 aa, Mus musculus
FIRNKANGYTTEFSASVMG

SEQ ID NO: 23 - DNA, 42 bp, Mus musculus
gatcccccctatggtaacccccattattatgctatggactac

SEQ ID NO: 24 - protein, 14 aa, Mus musculus
DPPYGNPHYYAMDY

SEQ ID NO: 25 - DNA, 333 bp, Mus musculus
  CDS (1)...(333)
gacattgtgctgacccagtctcctgcttccttagctgtttctctggggcagagggccaccatctcatgcagggccagcaaaagtgtcagtgcatctggctataattatatgcactggtaccaacagaaagcagggcagccacccaaactcctcatccatcttgcatccaacctagaatctggggtccctgccaggttcagtggcagtgggtctgggacagacttcaccctcaacatccatcctgtggaggaggaggatgcttcaacctattactgtcagcacagtggggagcttccattcacgttcggctcggggacaaagttggaaataaaa

SEQ ID NO: 26 - protein, 111 aa, Mus musculus
DIVLTQSPASLAVSLGQRATISCRASKSVSASGYNYMHWYQQKAGQPPKLLIHLASNLESGVPARFSGSGSGTDFTLNIHPVEEEDASTYYCQHSGELPFTFGSGTKLEIK

SEQ ID NO: 27 - DNA, 45 bp, Mus musculus
agggccagcaaaagtgtcagtgcatctggctataattatatgcac

SEQ ID NO: 28 - protein, 15 aa, Mus musculus
RASKSVSASGYNYMH

SEQ ID NO: 29 - DNA, 21 bp, Mus musculus
cttgcatccaacctagaatct

SEQ ID NO: 30 - protein, 7 aa, Mus musculus
LASNLES

SEQ ID NO: 31 - DNA, 27 bp, Mus musculus
cagcacagtggggagcttccattcacg

SEQ ID NO: 32 - protein, 9 aa, Mus musculus
QHSGELPFT

SEQ ID NO: 33 - DNA, 22 bp, Artificial
  Description of Artificial Sequence Primer
agttgaggggactttcccaggc